(19) United States
(12) Patent Application Publication
(10) Pub. No.: US 2026/0354359 A1
(43) Pub. Date: Nov. 12, 2026

(54) COMPOSITIONS AND METHODS FOR GENERATING AND USING PATIENT-SPECIFIC THYMUS-BEARING MICE

(71) Applicant: THE REGENTS OF THE UNIVERSITY OF COLORADO, A BODY CORPORATE, Denver, CO (US)

(72) Inventors: Holger A. RUSS, Denver, CO (US); Antonio JIMENO, Denver, CO (US)

(21) Appl. No.: 19/561,980

(22) Filed: Mar. 10, 2026

Related U.S. Application Data
(63) Continuation of application No. PCT/US2024/046269, filed on Sep. 11, 2024.
(60) Provisional application No. 63/537,693, filed on Sep. 11, 2023.

(51) Int. Cl. A01K 67/0278

(52) U.S. Cl. CPC A01K 67/0278; A01K 2217/072; A01K 2227/105; A01K 2267/03; A01K 2267/0331

(57) ABSTRACT

Embodiments of the instant disclosure relate to compositions and methods for generating and using humanized mice including, but not limited to, stem-cell derived thymic organoids (sTOs). In certain embodiments, a humanized mouse can be developed where T cells can be educated in a patient-derived sTO autologous to the patient providing a reliable, reproducible, and complete environment to study disorder-related immunity and patient-specific immunity. In certain embodiments, the subject has cancer, an autoimmune disorder, an immunocompromising disorder, or other disorder. In other embodiments, therapeutic agents can be assessed for efficacy to treat, for example, disorder-related immunity, cancer, and patient specific immunity using the humanized mouse.

### PRIORITY

This application is a Continuation Application of International PCT Application No. PCT/US2024/046269 filed Sep. 11, 2024, which claims the benefit of U.S. Provisional Application No. 63/537,693, filed on Sep. 11, 2023. These applications and all accompanying documents are incorporated herein by reference in their entireties for all purposes.

### GOVERNMENT FUNDING

This invention was made with government support under Grant No. R01CA149456 awarded by the National Institutes of Health. The government has certain rights in this invention.

### FIELD

Embodiments of the instant disclosure relate to compositions and methods for generating and using patient specific sTO-bearing mice. In certain embodiments, a mouse xenograft can be developed where T cells can be educated in a patient-derived implanted thymic organoid autologous to a patient-specific tumor providing a reliable, reproducible, and complete environment to study cancer immunity and patient specific cancer immunity.

### SEQUENCE LISTING

This application contains references to amino acid sequences and/or nucleic acid sequences which have been submitted concurrently herewith as the sequence listing.xml file entitled “126635-883274_SequenceListing.xml,” file size of 19,627 bytes, created on Mar. 6, 2026. This sequence listing is hereby incorporated by reference in its entirety for all purposes pursuant to 37 C.F.R. § 1.52(e)(5).

### BACKGROUND

Immunotherapy development has been hindered in part by the lack of preclinical modeling systems available for accurately mimicking tumor-stroma interactions to understand the tumor environment more thoroughly and for testing personalized therapy protocols. Humanized mouse (HM) models have demonstrated potential to fill this need but have not yet been fully developed for prolonged use and study. Therefore, a need exists for models to study cancer, autoimmune, and immune cell interactions for understanding cancer progression and for developing improved therapeutic interventions.

### SUMMARY

Embodiments of the instant disclosure relate to compositions and methods for generating and using stem cell-derived thymic organoids (sTOs)-bearing mice. In accordance with these embodiments, the sTO-bearing mice disclosed herein can be patient-specific for a disease or condition to assess therapeutically relevant agents or processes to treat the patient. In some embodiments, a patient specific sTO-bearing mice or other sTO-bearing mice can be generated to study cancer, an autoimmune disorder, an immunocompromising disorder, or other disorder (e.g., in a patient) and analyze effects of therapeutic agents on a targeted disorder and/or its progression in the disclosed mouse or mouse model of the disorder. In some embodiments, the disorder is cancer and/or a patient has cancer. In certain embodiments, a humanized mouse xenograft can be used to study where T-cells are educated in a subject-derived or patient-derived implanted thymic organoid autologous to a patient-specific sample (e.g., tumor) to provide a reliable, reproducible, and complete environment to study disorder-related immunity and patient specific immunity.

In certain embodiments, peripheral blood mononuclear cells (PBMCs) from a patient (e.g., having cancer or other disorder) can be reprogrammed into induced pluripotent stem cells (iPSCs) to generate stem cell-derived thymic organoids (sTOs). In other embodiments, it is contemplated that a close relative to a patient could also be used to create a humanized mouse model disclosed herein. Once obtained, sTOs can be implanted into immunocompromised, thymectomized HLA matched humanized mice (mHM). In certain embodiments, the mouse can be further engrafted with tissue (e.g., tumor tissue) from the same subject (autologous, mHMTA) or different subject (e.g., a close relative suffering from a condition where the mouse model is generated from a healthy relative). In certain embodiments, patient specific sTO-bearing mice can be used to assess presence or absence of thymic markers on developing T cells for detecting T cell maturation. In other embodiments, patient specific, sTO-bearing mice can be used to assess effects on the mouse of certain conditions that correlate with human disorders. In accordance with these embodiments, assessing effects of a T-cell activating agent, a T-cell maturation agent, an anti-cancer agent, an immunomodulatory agent, or other agent. In certain embodiments, effects of the agents on expression and/or progression of T cell maturation can be assessed.

In certain embodiments, a humanized mouse can include patient sTOs, where the humanized mouse has been further exposed to or engrafted with a sample derived from the patient HLA-matched to the sTOs. In certain embodiments, the sTOs include, but are not limited to, induced pluripotent stem cells (iPSCs) from the patient differentiated into hematopoietic stem and progenitor cells (HSPCs) where the patient has cancer. In other embodiments, the humanized mouse of use herein can include an immunocompromised mouse, a thymectomized mouse, or an immunocompromised, thymectomized mouse. In some embodiments, the humanized sTO-bearing mouse can be engrafted with at least one sample or administered at least one sample from the patient generating an autologous patient-specific humanized sTO-bearing mouse. In certain embodiments, the at least one engrafted or administered sample can be a tumor sample from the patient having cancer (e.g., at least one tumor sample from a melanoma patient).

In certain embodiments, sTOs of humanized mice can contain CD3⁺ or CD4⁺/CD8⁺ single or double positive T-cells. In yet other embodiments, sTOs of humanized mice can express thymic markers including, but not limited to, one or more of EPCAM (CD326), KRT5, and KRT8A. In accordance with these embodiments, T cells can be indicative of an ability of the humanized mouse to mimic the patient specific tumor environment for study and therapeutic evaluation. In other embodiments, circulating CD4⁺ T-cells of humanized mice disclosed herein can express at least one of CD45RA⁺ and CD45RO⁺ on circulating T-cells. In other embodiments, bone marrow of humanized mice can express an increased number of CD34⁺/45⁺ CD4⁺/CD8⁺ double positive T-cells compared to other humanized mice or other humanized mice not generated by methods disclosed in the instant application. In certain embodiments, at least one therapeutic agent can be tested on humanized mice disclosed herein for assessing effects of the at least one therapeutic agent on T-cell expansion and maturation in the humanized mice representative of use in the patient of which the humanized mouse was designed.

In certain embodiments, patients for which the humanized mice can be created can be cancer patients, patients having an autoimmune disorder, patients having an immunocompromising disorder, or patients having another disorder (e.g., where T-cell intervention treats the disorder). In accordance with some of these embodiments, the cancer can include a solid tumor. In certain embodiments, the solid tumor can include, but is not limited to, liver cancer, brain cancer, breast cancer, lung cancer, kidney cancer, pancreatic cancer, stomach cancer, esophageal cancer, skin cancer (e.g., melanoma, squamous skin cancer, basal cell skin cancer), colon cancer (e.g., high colon cancer, including microsatellite instability-high colon cancer), ovarian cancer, testicular cancer, head and neck cancer, bladder cancer, bone cancer, sarcoma, leukemia, lymphoma, and other solid tumor. In other embodiment, the cancer can include a non-solid tumor. In certain embodiments, a non-solid tumor can include, but is not limited to, blood-related cancers or a bone-related cancers or the like. In certain embodiments, the patient has a solid tumor. In some embodiments, the cancer is melanoma. In accordance with some of these embodiments, the autoimmune disorder, immunocompromising disorder, or other disorder can include, but is not limited to diabetes (e.g., type 1 diabetes), arthritis (e.g., rheumatoid arthritis (RA)), systemic lupus erythematosus (SLE), multiple sclerosis (MS), Hashimoto's thyroiditis, Graves' disease, psoriasis, inflammatory bowel disease (IBD), Sjögren's disease, Addison's disease, myasthenia gravis, autoimmune hepatitis, celiac disease, vitiligo, and pemphigus vulgaris, or a combination thereof.

In certain embodiments, methods for generating humanized patient specific sTO-bearing mice are disclosed. Some embodiments relate to methods for generating humanized patient specific mice include, but are not limited to, introducing sTOs from a patient to immunocompromised and/or thymectomized humanized mice, where the humanized mice can be HLA matched to the sTO. In other embodiments, the humanized mouse can be engrafted with at least one sample (e.g., tissue) from the patient (e.g., tumor cells, tumor tissue, iPSC derived tissues, or other tissue) which is the same or different subject from which the sTOs are derived. In other embodiments, a patient's sTOs can be generated by obtaining PBMCs from the patient, converting the PBMCs into iPSCs and further differentiating the iPSCs to TEPs and combining the TEPs with HSPCs to differentiate the TEPs into thymic epithelial cells (TECs) to form sTOs. In some embodiments, the patient can have cancer, an autoimmune disorder, an immunocompromising disorder, or other disorder. In other embodiments, methods further include where TECs are generated using an sTO culture system. In certain embodiments, the PBMCs include, but are not limited to, PBMCs for generating autologous thymic tissue matched to patient tumor tissue. PBMCs can be obtained from an individual other than the patient so long as the subject and patient are HLA matched. In accordance with this embodiment, HLA matching contemplated herein can be matching class I and/or class II.

In certain embodiments, cells and/or tissue isolated from the patient-specific humanized mouse model can be used for further investigation and study. In accordance with these embodiments, cells and/or tissue can be stained by methods known in the art to assess how a therapeutic agent targets and affects the cells and/or tissue (e.g., engrafted tumor tissue or autoimmune-related tissue). In certain embodiments, these studies can include, but are not limited to, whole cell assessments, protein presence and/or expression, nucleic acid polynucleotide levels or presence, modification, and/or expression, lipid analysis, and/or sugar analysis.

In certain embodiments, methods for evaluating efficacy of therapeutic agents to treat a patient having a health condition, including, but not limited to, administering a therapeutic agent to patient-specific humanized mice and determining efficacy of the therapeutic agent to treat the health condition. In accordance with these embodiments, determining success of therapeutic agent in the patient-specific humanized mouse can be indicative of success of the agent to treat the patient. In some embodiments the health condition can include cancer, an autoimmune disorder, an immunocompromising disorder, and/or other disorder. In certain embodiments, therapeutic agents tested using the disclosed patient-specific humanized mouse can be used to assess tumor growth, expansion, and/or metastasis. In other embodiments, a therapeutic agent can include a combination of therapeutic agents including experimental and/or those known in the art.

In some embodiments, kits are disclosed. In accordance with these embodiments, kits for generating a patient-specific humanized mouse model can include a composition including sTOs (e.g., patient specific sTOs); a sample HLA (e.g., tissue sample from a patient) matched to the sTOs; and at least one container. In other embodiments, a kit can further include at least one device for delivering at least one of the composition and sample to an immunocompromised, thymectomized mouse. In some embodiments, sTOs and sample are from the same patient or from different subjects (e.g., a related subject to a patient).

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIG. 1A represents a schematic diagram illustrating a general method for differentiation of patient-derived iPSCs to TEPs and subsequent production of sTOs, according to various embodiments of the disclosure.

FIGS. 1B-1C represents exemplary plots indicating expression levels of representative thymic markers (1B) or tissue restricted antigen (TRA) as a measure of AIRE activity (1C) in patient-specific sTO cultures at different timepoints, according to various embodiments of the disclosure.

FIG. 1D represents exemplary immunofluorescent images of sTOs after approximately three weeks of culture, according to various embodiments of the disclosure.

FIG. 1E represents exemplary flow cytometry plots (top two rows) and quantification (bottom row) of dissociated sTOs to identify CD45⁺/CD5⁺/7⁺ T cells (top row) and CD45⁺/CD3⁺/TCRαβ CD4⁺/8⁺ T cells, according to various aspects of the disclosure.

FIG. 2A represents a schematic diagram of mHMTA production, according to various embodiments of the disclosure.

FIG. 2B represents an exemplary image of a successfully engrafted sTO growing under the kidney capsule, indicated by the arrow, according to various embodiments of the disclosure.

FIG. 2C represents exemplary immunohistochemical images of human thymic tissue with engrafted sTO tissue, according to various embodiments of the disclosure.

FIG. 2D represents exemplary cytometry plots and immunohistochemistry images of dissociated tissue from engrafted sTOs, according to various embodiments of the disclosure.

FIG. 2E represents exemplary multiplexed ion beam imaging (MIBI) images of sTOs expressing IDO-1, Podoplanin, T-bet, and Tim3 (top rows) as well as exemplary images of sTO-associated T cells (bottom rows), with the bottom most image of a nongrafted mouse kidney provided for comparison of all markers, according to various embodiments of the disclosure.

FIGS. 3A-3D represent exemplary plots comparing the percentage of human CD45⁺ human cells in bone marrow (3A) and human T-cells in blood, spleen, and tumor of control mHM and mHMTA (sTO) mice (3B, 3C, and 3D, respectively), according to various embodiments of the disclosure.

FIG. 3E represents exemplary cytometric plots of CD34⁺/45⁺ cells in the bone marrow, and the CD3⁺/45⁺ T-cells in the peripheral blood, spleen, and sTO tissue of a mHMTA mouse, according to various embodiments of the disclosure.

FIG. 3F represents exemplary cytometric plots of splenic T-cell populations in representative mHM vs. a representative mHIMTA, according to certain embodiments of the disclosure.

FIG. 3G represents an exemplary plot depicting levels of HLA-DR⁺ T cells vs. HLA-DR-T cells in mHM and mHMTA animals, according to certain embodiments of the disclosure.

FIGS. 4A-4B represents exemplary plots depicting normalized melanoma tumor growth in NSG mice (4A) and a direct comparison of all mice at days 22 and 25 post inoculation (4B), according to certain embodiments of the disclosure.

FIGS. 4C-4D represents exemplary plots depicting melanoma tumor growth in NSG mice (4C) and a direct comparison of all mice at days 22 and 25 post inoculation (4D), according to certain embodiments of the disclosure.

FIGS. 5A-5B represent exemplary plots depicting tumor growth in mHM and mHMTA mice after patient-derived xenograft (PDX), plotted as on a log-transformed axis per week post-PDX (5A) or as a fold change at day 30 post-PDX (5B), according to certain embodiments of the disclosure.

FIGS. 5C-5D represent exemplary plots depicting tumor growth in an exemplary mHMTA mouse (provided as red in original) compared to an mHM animal (provided as green in original) (5C) as well as Bayesian analysis of the evidence (5D), according to certain embodiments of the disclosure.

FIG. 5E represents exemplary immunohistochemistry images of mHM and mHM_{TA} tumors showing CD3⁺/45⁺ T cell infiltration, according to certain embodiments of the disclosure.

FIG. 5F represents exemplary cytometry plots of tumors in an mHM_{TA} mouse and a comparably humanized mHM, according to certain embodiments of the disclosure.

FIG. 6A represents three exemplary single cell sequencing UMAP plots colored by Azimuth pipeline cell type predictions, according to certain embodiments of the disclosure.

FIG. 6B represents an exemplary plot of deep exome sequencing of PDX tissue showing distribution of variant allele frequencies (VAFs) in mHM, mHM_{TA}, and CUHM009F0 tumor, according to certain embodiments of the disclosure.

FIG. 6C represents an exemplary heatmap of mHM and mHM_{TA} variants sorted using binomial logistic modeling of VAFs to identify subclonal variants, according to certain embodiments of the disclosure.

FIG. 6D represents an exemplary plot of predicted neoantigens plotted against predicted HLA binding affinity for peptides derived from each variant, where the dashed line represents affinity cutoff to distinguish strong binders below the line, according to certain embodiments of the disclosure.

FIGS. 7A-7B represents an exemplary schematic of iPSC generation from patient blood (7A) and exemplary cytometry plots illustrating expression of representative pluripotent markers (7B), according to certain embodiments of the disclosure.

FIGS. 8A-8B represents exemplary plots depicting fractions of different cell populations in sTO cultures at indicated weeks after introducing HSPCs to TEP cultures (8A) and exemplary cytometry plots depicting these cell populations (8B), according to certain embodiments of the disclosure.

FIGS. 9A-9B represents exemplary flow cytometry plots illustrating presence of CD45⁺ and CD3⁺ cells of NSG, NSG with sTO and thymectomized humanized mice (HM) according to certain embodiments of the disclosure.

FIG. 10 represents exemplary flow cytometry plots illustrating UCB120 and UCB122 HSPCs expanded from cord blood samples, according to certain embodiments of the disclosure.

FIGS. 11A-11B represents exemplary plots depicting the number of human CD45⁺ cells per live cells (11A) as well as the number of CD34⁺ HSPCs in all human CD45⁺ bone marrow cells (11B) in bone marrow of mHM and mHM_{TA} mice, according to certain embodiments of the disclosure.

FIG. 12 represents an exemplary plot depicting fractions of circulating human lymphocytes (CD45⁺) cells among all live cells at different weeks post HSC injection in mHM and mHM_{TA} animals, according to certain embodiments of the disclosure.

FIG. 13 represents an exemplary plot depicting fractions of circulating human T cells (CD3⁺ cells) among all live cells at different weeks post HSC injection in mHM and mHM_{TA} animals, according to various aspects of the disclosure.

FIGS. 14A-14B represent exemplary immunohistochemistry images (14A) and flow cytometry plots (14B) of additional sTOs identified in mHM_{TA}, according to certain embodiments of the disclosure.

FIGS. 15A-15B represents exemplary immunohistochemistry images (15A) of murine thymus tissue from mHM illustrating expression of only murine cytokeratins (KRT5 and KRT8) as well as CD3⁺/CD45⁺ and CD4⁺/8⁺ DP T-cells along with cytometry (15B) of the murine thymus tissue verifying presence of CD3⁺ T cells, according to certain embodiments of the disclosure.

FIG. 16 represents exemplary cytometry plots illustrating splenocyte CD8⁺ T-cell activation in mHM and mHM_{TA} animals (assessed by CD45RO⁺/HLA-DR⁺ cells in the spleen), according to certain embodiments of the disclosure.

### DEFINITIONS

Terms, unless defined herein, have meanings as commonly understood by a person of ordinary skill in the art relevant to certain embodiments disclosed herein or as applicable.

As used herein “about” unless otherwise indicated, applies to all numbers expressing quantities of agents and/or compounds, properties such as molecular weights, reaction conditions, and as disclosed herein are contemplated as being modified in all instances by this term. Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis, or therapy is desired, for example, humans, pets, livestock, horses or other animals. The term “patient” as used herein can refer to any mammal, including but not limited to, a non-human primate (for example, a monkey or great ape), livestock or pets such as a cow, a pig, a cat, a dog, a rat, a mouse, a horse, a goat, a rabbit, a sheep, a hamster, a guinea pig) or other subject. In some embodiments, the mammalian patient is a human such as an adult, a young child, adolescent, toddler, infant or fetus. In some embodiments, the patient can be a human, including but not limited to an older adult having an age of about 40 years or more, an age of about 50 years or more, an age of about 60 years or more, or an age of about 70 years or more. In other embodiments, the patient can be a young patient such as a toddler, child, or adolescent.

As used herein, “pluripotent stem cell” or “pluripotent cell” can refer to a cell capable, under appropriate conditions, of producing progeny of several different cell types that are derivatives of all three germinal layers (i.e., endoderm, mesoderm, and ectoderm) or reprogrammed. Examples of pluripotent stem cells (PSC) include, but are not limited to, embryonic stem (ES) cells, embryonic germ stem (EG) cells, induced pluripotent stem (iPSC) cells, adult stem cells, and the like. PSCs can be from any organism of interest, including but not limited to, primate, (e.g., human), canine, feline, murine, equine, porcine, avian, camel, bovine, ovine, and the like.

As used herein, “marker” can refer to any molecule that can be measured or detected, for example. In certain embodiments disclosed herein, a marker can include, without limitations, a nucleic acid, a transcript of a gene, a polypeptide product of a gene, a polypeptide, a protein, a glycoprotein, a carbohydrate, a glycolipid, a lipid, a lipoprotein, a carbohydrate, and/or a small molecule. As used herein, “expression” and grammatical equivalents thereof, in the context of a marker, can refer to production or transcription or translation of the marker. In addition, level or amount of the marker can be assessed and compared to controls to evaluate a process.

As used herein, “humanized mouse” can refer to a mouse with engrafted or implanted human tissue. The engrafted or implanted tissue can be a sample from a human tumor. Human tumor can be from a biopsy or other sample of a cancer obtained from a patient. Human tumor can be a cell line derived from a human cancer or tumor. Alternatively, human tissue can be a healthy and/or normal tissue (e.g., non-cancerous tissue) from a human or a collection of cells obtained from a human, such as sTOs described herein.

As used herein, “sTO culture system” can refer to a system for generating sTOs from subject or patient-specific thymic epithelial cells (TECs). In certain embodiments, compositions, and methods for generating sTOs can be as described in US 2022/0041988, and Ramos, et al. (Stem Cell Reports, 2023 Apr. 11; 18(4): 829-840) incorporated herein by reference in their entireties for all purposes. Other methods for generating subject specific sTOs known in the art are also contemplated of use herein.

As used herein, the term “medium” or “media” used in context of cell culture or the phrase “cell culture medium” or “cell medium” can refer to a cellular growth medium suitable for culturing of cells of the present disclosure. A “base medium,” as referred to herein, can be low protein or enriched protein-containing medium. Base medium can be a cell culture medium that has not been modified by adding one or more additives/supplements. Non-limiting examples of a base medium suitable for use herein can include, but are not limited to, MEM alpha media, Minimum Essential Medium (MEM), Eagle's Medium, Dulbecco's Modified Eagle Medium (DMEM), Dulbecco's Modified Eagle Medium: Nutrient Mixture F-12 (DMEM/F12), F10 Nutrient Mixture, Ham's F10 Nutrient Mix, Ham's F12 Nutrient Mixture, Medium 199, RPMI, RPMI1640, reduced serum medium, basal medium (BME), DMEM/F12 (1:1), Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E and the like, and combinations thereof.

### DETAILED DESCRIPTION

In the following sections, certain exemplary mice, compositions, and methods are described in order to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

Immunotherapy development has been hindered by the lack of preclinical model systems available for accurately modeling tumor-stroma interactions to further research and assess potential interventions. Humanized mice have demonstrated potential to fill this need. Human hematopoietic stem and progenitor cells, derived from either donated umbilical cord blood (UCB) or the peripheral blood of adults treated with filgrastim, colonize mouse bone marrow, and give rise to B-cell, monocyte, and T-cell lineages. While myeloid lineages do not require developmental stimuli beyond those which are present in the bone marrow, progenitor T (pro T) cells migrate to the thymus where they undergo T-cell lineage commitment and T-cell receptor (TCR) recombination, followed by positive and negative selection to produce mature T-cells. In a humanized mouse model, CD34⁺/CD7⁺/CD5⁺ pro T-cells traffic to the murine thymus before moving systemically. In some cases, these T-cells can be activated by immune checkpoint inhibitors (ICIs) and specifically attack tumor cells, but such activity appears stochastic. One possible reason for the lack of correlative response in humanized mouse PDX is that the TCR repertoire of the human T-cells educated by a murine thymus differs from that produced by a human thymus. Prior studies have demonstrated that humanized mouse T-cells are often anergic and incapable of responding properly to foreign antigens or tumor-associated neoantigens.

Further, previously described humanized mouse PDX models have been engrafted with HSPCs from cord blood, and their resulting immune cells are allogeneic to subsequently implanted patient tumors. HLA-matching and transgenic models attempt to minimize tissue mismatches, but matching is often restricted, if at all, to only the HLA-A and HLA-B alleles. Several models have been described in which donor or patient PBMCs are used to humanize mice, and tumors are subsequently implanted. Although this approach can be successful, these mice rapidly succumb to graft versus host disease (GvHD), limiting their usefulness in extended tumor treatment studies. In addition, there have been isolated reports of humanized mouse being generated from surgically harvested bone marrow of cancer patients, but this type of model requires intrusive surgery to produce.

In response to this obstacle, several groups have implanted fetal thymus into these humanized mouse models to produce a more representative T-cell repertoire. Although this and other similar strategies using neonatal thymic tissue together with cord blood HSPCs can be effective, thymic tissue is not readily available, and even when used, resultant T-cells have been educated in a thymic environment allogeneic to the tumor. Previously, a direct differentiation protocol was demonstrated to generate thymic epithelial progenitor cells (TEPs) from multiple iPSC lines. (e.g., as found in US 2022/0041988 incorporated herein by reference in its entirety for all purposes) Upon transplantation into athymic nude mice, TEPs further differentiated into functional TECs with the ability to educate mouse T-cells. Additionally, while differentiation of TEPs from human iPSCs has been demonstrated, further development has been directed with a goal of generating functional TECs in vitro. The generation of thymic tissue autologous to any patient via iPSC technology will provide a reliable format for studying cancer and targeting intervention and treatments. Embodiments disclosed herein overcome many of these issues with other models.

Embodiments of the instant disclosure relate to compositions and methods for generating and using humanized mice. In accordance with these embodiments, humanized mice can include sTOs, where humanized mice can be HLA-matched to the sTOs. In certain embodiments, humanized mice can further include, at least one tissue sample from a patient HLA-matched to the sTO engrafted or otherwise introduced to the humanized mouse. In some embodiments, humanized mice can be generated to study cancer, autoimmune disorders, immunocompromising disorders, or other disorders in a subject (e.g., human patient). In other embodiments, humanized mice can be generated to assess effects of therapeutic agents on cancer, autoimmune disorders, immunocompromising disorder, or other disorders or to study disease progression or inhibition in the subject using the humanized mouse. In certain embodiments, the humanized mouse disclosed herein can be used as a model to study where T-cells are educated in an sTO autologous to a tissue (e.g., a tumor) from the patient or another subject, HLA-matched with the patient. In accordance these embodiments, humanized mice can provide, for example, a reliable, reproducible, and complete environment to study cancer immunity, cancer treatment, autoimmunity, and/or immune regulation in patient-specific manners. In some embodiments, HLA of humanized mice can be matched to sTOs derived from a patient, or another subject having identical or similar HLA as the patient. In certain embodiments where the sTOs are derived from a subject who is not a patient, a source of sTOs can be a subject having sufficient similarity to the patient such that the T cells generated by the sTOs from the subject can recognize tissue from the patient as “self” not as “foreign”. In accordance with these embodiments, similarity between source of sTOs and HLA of the humanized mouse can be at least 50% similarity (or match), at least 55% similarity, at least 60% similarity, at least 65% similarity, at least 70% similarity, at least 75% similarity, at least 80% similarity, at least 85% similarity, at least 90% similarity, at least 95% similarity, at least 96% similarity, at least 97% similarity, at least 98% similarity, or at least 99% similarity, or more. In yet other embodiments, similarity between source of sTOs and HLA of the humanized mouse can be about 100% similarity or 100% similarity. In some embodiments, a source of sTOs can be derived from a parent, sibling (e.g., twin), or child or other relative having the same or similar HLA as the patient. In certain embodiments, the HLA, for example, to be matched can be human MHC class I (e.g., HLA-A, HLA-B, HLA-C, non-classical HLA class I, isoforms thereof or other HLA class I, or a combination thereof). In other embodiments, the HLA can be human MHC class II (e.g., HLA-DP, HLA-DM, HLA-DOA, HLA-DOB, HLA-DQ, HLA-DR, non-classical HLA class II, isoforms thereof or other HLA class II, or a combination thereof). In other embodiments, the HLA can be a combination of MHC class I and MHC class II.

In certain embodiments, a patient can have cancer. In accordance with these embodiments, the cancer can be a solid tumor or a non-solid tumor. In addition, the cancer can be capable of metastasizing or has previously metastasized. In certain embodiments, the cancer can include, but is not limited to, skin cancer, head and neck cancer, liver cancer, brain cancer, breast cancer, lung cancer, mesothelioma, thymoma, bladder cancer, kidney cancer, testicular cancer, prostate cancer, pancreatic cancer, cholangiocarcinoma, stomach cancer, esophageal cancer, small intestine cancer, colon cancer (or colorectal cancer), peritoneal cancer, ovarian cancer, fallopian tube cancer, endometrial cancer, cervical cancer, sarcoma, leukemia, lymphoma, myeloma, myeloma, myeloproliferative disorders, neuroendocrine cancer, undifferentiated cancers, and cancers of unknown origins. In some embodiments, skin cancer can include, but is not limited to, melanoma, squamous skin cancer, basal cell carcinoma or other skin cancer. In other embodiments, a solid tumor can be microsatellite instability-high colon cancer.

In certain embodiments, sTOs can be prepared from PBMCs from a patient. PBMCs can be dedifferentiated and converted into iPSCs and then differentiated into TECs to generate sTOs. In certain embodiments, patients providing the PBMCs to generate the sTOs have cancer, an autoimmune disorder, an immunocompromising disorder, or other disorder. In further embodiments, once obtained, these sTOs can be implanted in immunocompromised, HLA-matched humanized mice (mHM), or in a thymectomized, HLA-matched humanized mouse (mHM_{TA}). In certain embodiments, sTOs can be engrafted into the mouse, including, but not limited to, engraftment on one or more of a kidney capsule, intramuscular, subcutaneously, omentum, in liver lobes or the like or other location for engraftment. In further embodiments, humanized mice of use herein can be immunocompromised, thymectomized, or both.

In certain embodiments, humanized mice can be engrafted with at least one tissue sample from a patient. In some embodiments, the at least one tissue samples can be from a tumor in the patient having cancer. In accordance with these embodiments, the tumor can include, but is not limited to, skin cancer, head and neck cancer, liver cancer, brain cancer, breast cancer, lung cancer, mesothelioma, thymoma, bladder cancer, kidney cancer, testicular cancer, prostate cancer, pancreatic cancer, cholangiocarcinoma, stomach cancer, esophageal cancer, small intestine cancer, colon cancer (or a colorectal cancer), peritoneal cancer, ovarian cancer, fallopian tube cancer, endometrial cancer, cervical cancer, sarcoma, leukemia, lymphoma, myeloma, myeloma, myeloproliferative disorders, neuroendocrine cancer, undifferentiated cancers, and cancers of unknown origins. In some embodiments, skin cancer can include, but is not limited to, melanoma, squamous skin cancer, basal cell carcinoma or other skin cancer. In other embodiments, a solid tumor can be microsatellite instability-high colon cancer. In some embodiments, the at least one tissue samples can be from a patient having an autoimmune disorder, an immunocompromising disorder, or another disorder. In accordance with these embodiments, the at least one tissue samples for engrafting into humanized mice can be tissue affected by the disorder (e.g., diseased tissue or tissue affected by the condition). In some embodiments, the at least one tissue sample can be pancreatic tissue from a patient suffering from diabetes (e.g., type 1 diabetes) for engrafting into humanized mice. In some embodiments, the engrafted tissue and sTOs can be from the same patient. In other embodiments, humanized mice can have HLA-A, HLA-B, HLA-C, non-classical HLA, isoforms thereof or other HLA, or a combination of HLA class I molecules and/or HLA-DP, HLA-DM, HLA-DOA, HLA-DOB, HLA-DQ, HLA-DR, non-classical HLA class II, isoforms thereof or other HLA class II, or combination of HLA class I and/or class II molecules. In accordance with these embodiments, engrafted tissue and sTOs can be HLA-matched to the patient. In some embodiments, humanized mice disclosed herein can express HLA-A, HLA-B, and/or HLA-C. In some embodiments, humanized mice disclosed herein can express HLA-DP, HLA-DM, HLA-DOA, HLA-DOB, HLA-DQ, and/or HLA-DR

In certain embodiments, humanized mice can be used to assess presence and/or absence of thymic markers for detecting T cell maturation produced by the subject or patient-specific sTOs. In some embodiments, sTOs of the humanized mouse can express CD4⁺/CD8⁺ double positive T cells or CD3⁺ single positive T cells. In yet other embodiments, sTOs in humanized mice can express thymic markers, including, but not limited to, one or more of EPCAM, KRT5, KRT8A, or any combination thereof. In some embodiments, CD4⁺ T cells expressing either CD45RA⁺, CD45RO⁺, or both can be present in humanized mice. In some embodiments, humanized mice can express an increased number of CD34⁺/CD45⁺, CD4⁺/CD8⁺ double positive T cells as compared to another humanized mouse or a control humanized mouse (e.g., a humanized mouse without engrafted patient-specific sTOs). In accordance with these embodiments, a control humanized mouse can include a control humanized mouse absent partial or complete HLA matching.

In certain embodiments, any strain of mouse can be used to create humanized mice disclosed herein. For example, strains of mouse for generating a humanized mouse disclosed herein can include, but are not limited to, C57BL/6, BALB/c, DBA, NOD, B6, nude, CBA/J, Swiss Webster, NMRI, or other mice. In certain embodiments, humanized mice of use herein can be NOD.Cg-Prkdcscid Il2rgtm 1Wjl/SzJ (NSG) mice and strains derived from NSG mice. In other embodiments, the mouse can be immunocompromised (e.g., the mouse native immune system can be reduced, destroyed, or eliminated) prior to implantation or engraftment of sTOs and/or at least one tissue sample contemplated herein by any method known in the art. In some embodiments, immunocompromised mice can be prepared by myeloablative conditioning via total body irradiation (e.g., with gamma (γ) irradiation) or chemically (e.g., with busulfan). In certain embodiments, mice can be thymectomized by methods known in the art prior to implantation or engraftment of the sTOs and/or at least one tissue sample from a subject (e.g., patient). In certain embodiments, the mouse can be both immunocompromised and thymectomized prior to implantation or engraftment of sTOs and/or at least one tissue sample from a subject (e.g., patient).

In some embodiments, cells and/or at least one tissue sample can be isolated from humanized mice to study a subject for which the humanized mouse is a representative model. In certain embodiments, cells and/or at least one tissue sample can include, but is not limited to, patient tissue previously engrafted into the humanized mouse. In accordance with these embodiments, the cells and/or at least one tissue sample can be obtained from a tumor or cells previously introduced to the humanized mouse and include at least one of the skin cancer, head and neck cancer, liver cancer, brain cancer, breast cancer, lung cancer, mesothelioma, thymoma, bladder cancer, kidney cancer, testicular cancer, prostate cancer, pancreatic cancer, cholangiocarcinoma, stomach cancer, esophageal cancer, small intestine cancer, colon cancer (or colorectal cancer), peritoneal cancer, ovarian cancer, fallopian tube cancer, endometrial cancer, cervical cancer, sarcoma, leukemia, lymphoma, myeloma, myeloma, myeloproliferative disorders, neuroendocrine cancer, undifferentiated cancers, and cancers of unknown origins and isolated before, during, or after a period of time (e.g., a therapeutic agent is administered to the humanized mouse). In certain embodiments, cells and/or at least one tissue sample can be a skin cancer and can include, but is not limited to, melanoma, squamous skin cancer, basal cell skin cancer, or other skin cancer or a combination thereof. In certain embodiments, cells and/or at least one tissue sample can be from colon cancer, including, but not limited to, microsatellite instability-high colon cancer. In an alternative embodiment, cells and/or at least one tissue sample isolated from the humanized mouse can include those isolated from the sTOs. In other embodiments, cells and/or at least one tissue sample can be other than the engrafted tissue or sTO (e.g., native mouse tissue). In some embodiments, cells and/or at least one tissue sample can be used for additional assays; for example, assays that determine presence, absence, and/or number of human T cells (either CD4⁺ T cells or CD8⁺ T cells or CD4⁺/CD8⁺ double positive T cells) educated in the humanized mouse sTOs. In other embodiments, cells and/or at least one tissue sample isolated from humanized mice can be assayed to determine specificity and/or cytotoxicity of the human T cells educated in sTOs, for example, to certain therapeutic agents or conditions. In some embodiments, isolated cells and/or at least one tissue sample can be assayed for the presence of immunoregulatory T cells (e.g., regulatory T cells or Tregs). In other embodiments, circulating CD4⁺ T-cells of the humanized mouse disclosed herein can express at least one of CD45RA⁺ and CD45RO⁺ on the circulating T-cells demonstrating differentiation of the T-cells. In yet other embodiments, bone marrow of humanized mice disclosed herein can be assessed for expression of, and/or increased number of, CD34⁺/CD45⁺ CD4⁺/CD8⁺ T-cells compared to other humanized mouse models or in response to a therapeutic agent administered to the humanized mouse to test efficacy of the therapeutic agent. In accordance with these embodiments, mature T cells of humanized mice disclosed herein can be indicative of ability of the humanized mice to mimic patient-specific tumor environments for study, evaluation, and therapeutic intervention and assessment.

In some embodiments, mice can be humanized by delivering cord blood HLA-matched to that of patient PBMCs, after which the patient's tumor tissue or other tissue was implanted. In some embodiments, to ensure the T-cells are not trafficked to the murine thymus, mice can be thymectomized prior to humanization with the matched cord blood. In accordance with disclosed embodiments, using humanized mice, sTO-educated T-cells can respond to antigenic stimulation, delay autologous PDX tumor growth, ameliorate disease symptoms or the disease condition, and/or cause genomic and/or transcriptomic changes in autologous PDX tissue consistent with immunoediting making humanized mice a useful tool for research and therapeutic evaluation.

In certain embodiments, humanized mice can have sTOs derived from PBMCs that were converted to iPSCs and subsequently differentiated to TECs to form sTOs. In some embodiments, humanized mouse models disclosed herein can include, but are not limited to, sTOs generated from PBMCs from a patient where the patient has cancer, an autoimmune disorder, an immunocompromising disorder, or other disorder. In some embodiments, humanized mice disclosed herein includes sTOs derived from PBMCs from patients with cancer.

In certain embodiments, humanized mice can include, but are not limited to, sTOs, where humanized mice can be humanized with a sample derived from a patient HLA-A matched to the sTOs. In certain embodiments, the sTOs can be generated from PBMCs from the patient where the patient has cancer. In an alternative embodiment, humanized mice can be HLA-A, HLA-B and/or HLA-C matched. In other embodiments, humanized mice can include an immunocompromised, thymectomized mouse. In yet other embodiments, humanized mice can be engrafted with at least one HLA-matched tissue sample from the patient of which the sTOs were derived generating an autologous patient-specific humanized mice.

In certain embodiments, sTOs of humanized mice express CD4⁺/CD8⁺ double positive (DP) T-cells. In yet other embodiments, sTOs of humanized mice can express thymic markers including, but not limited to, one or more of EPCAM, KRT5, and KRT8A, indicative of human T-cell selection activities.

In some embodiments, kits are contemplated for use to generate humanized mice disclosed herein. In certain embodiments, the kit for generating a humanized mouse can include, but not limited to, a composition with sTOs, a sample HLA-matched to the sTOs, and at least one container. In certain embodiments, kits can be for use in creating humanized mice for studying cancer, an autoimmune disorder, an immunocompromising disorder, or other disorder in a subject and/or for assessing efficacy of a treatment regimen in the patient. In some embodiments, the kit can further include at least one device for delivering at least one of the compositions and sample to an immunocompromised and/or thymectomized mouse. In some embodiments, sTOs and samples can be from the same subject (e.g., the patient). In alternative embodiments, sTOs and samples can be from different people. In further embodiments, samples can be from a patient having cancer, an autoimmune disorder, an immunocompromising disorder, or other disorder, and sTOs can be from another subject (e.g., the patient's parent, sibling, or child). In some embodiments, the kit can further include instructions for use in accordance with any of the methods described herein.

In some embodiments, kits can be in suitable packaging. Suitable packaging for storing, transport, and/or delivery of compositions disclosed herein includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle).

In certain embodiments, methods for generating a humanized patient specific mouse model are disclosed. Some embodiments relate to methods for generating the humanized patient specific mouse model include, for example, introducing sTOs to an immunocompromised and/or thymectomized mouse. In some embodiments, sTOs described above can be engrafted into humanized mice. In other embodiments, the sTOs can be engrafted into the kidney capsule, intramuscularly, subcutaneously, omentum, in liver lobes or the like, or at another location. In some embodiments, humanized mice can be engrafted with tissue (e.g., tumor, autoimmune-related tissue, or other tissue) from the patient. In some embodiments, the patient can be a cancer patient, where cancer can be skin cancer, head and neck cancer, liver cancer, brain cancer, breast cancer, lung cancer, mesothelioma, thymoma, bladder cancer, kidney cancer, testicular cancer, prostate cancer, pancreatic cancer, cholangiocarcinoma, stomach cancer, esophageal cancer, small intestine cancer, colon cancer, peritoneal cancer, ovarian cancer, fallopian tube cancer, endometrial cancer, cervical cancer, sarcoma, leukemia, lymphoma, myeloma, myeloma, myeloproliferative disorders, neuroendocrine cancer, undifferentiated cancers, and cancers of unknown origins. of the patient. In certain embodiments, cells and/or at least one tissue sample can be a skin cancer and can include, but is not limited to, melanoma, squamous skin cancer, basal cell skin cancer, or other skin cancer. In certain embodiments, cells and/or at least one tissue sample can be from colon cancer, (e.g., a microsatellite instability-high colon cancer). In other embodiments, sTOs engrafted or delivered to the humanized mouse can be generated from patient PBMCs converted to iPSCs differentiated into TECs to form sTOs. In some embodiments, methods can include, but are not limited to, TECs generated using an sTO culture system (previously disclosed). In certain embodiments, PBMCs include, but are not limited to, PBMCs for generating autologous thymic tissue matched to patient tumor tissue.

In certain embodiments, methods for evaluating efficacy of a therapeutic agent to treat a patient having a health condition are disclosed. In certain embodiments, humanized mice can be administered at least one therapeutic agent to a patient-specific humanized mouse and efficacy of the therapeutic agent to treat the health condition can be evaluated by methods disclosed herein. In some embodiments, the method includes, but is not limited to, administering a therapeutic agent to a humanized mouse to determine the efficacy of the therapeutic agent to enhance T-cell maturation or evaluate effects on at least one tissue sample engrafted on the humanized mouse. In certain embodiments, humanized mice can include sTOs and which can be HLA-matched to the patient-derived tissue sample. In some embodiments, humanized mice can further include at least one engrafted tissue that is from the patient HLA-matched to the sTOs. In yet other embodiments, the health condition can be at least one of cancer, an autoimmune disorder, an immunocompromising disorder, or other disorder. In certain embodiments, the health condition can be cancer, including, but not exclusively, one or more of skin cancer, head and neck cancer, liver cancer, brain cancer, breast cancer, lung cancer, mesothelioma, thymoma, bladder cancer, kidney cancer, testicular cancer, prostate cancer, pancreatic cancer, cholangiocarcinoma, stomach cancer, esophageal cancer, small intestine cancer, colon cancer, peritoneal cancer, ovarian cancer, fallopian tube cancer, endometrial cancer, cervical cancer, sarcoma, leukemia, lymphoma, myeloma, myeloma, myeloproliferative disorders, neuroendocrine cancer, undifferentiated cancers, and cancers of unknown origins. In certain embodiments, the skin cancer can include, but is not limited to, melanoma, squamous skin cancer, basal cell skin cancer, or other skin cancer. In certain embodiments, the cancer can be colon cancer and can include, but is not limited to, a microsatellite instability-high colon cancer. In other embodiments, the cancer is skin cancer and can be melanoma. In other embodiments, methods can include steps for determining the efficacy of a therapeutic agent to treat cancer contemplated herein and the humanized mouse can be engrafted with at least one tissue sample or cells from a tumor or circulating cancer. In certain embodiments, patients can have cancer and efficacy of the therapeutic agent can be determined by demonstrating whether the therapeutic agent reduces tumor growth, expansion, and/or metastasis of the engrafted tumor or tissue sample or cells in the humanized mouse representative or correlative of treating the patient for the cancer.

In other embodiments, the therapeutic agent can include a standard anti-cancer agent to assess efficacy of treatment. In an embodiment, the therapeutic agent can be a combination of two or more therapeutic compounds or therapeutic agents or biologics (e.g., antibody, antibody fragment thereof, or other biologic for treating a health condition). In certain embodiments, the therapeutic agent can include a standard, a new or experimental agent (e.g., anti-cancer agent, anti-autoimmune agent) where the humanized mouse model disclosed herein can be used to assess short and/or long-term efficacy of the standard/known, new, or experimental agent on the patient. In some embodiments the health condition can include cancer and the therapeutic agent can include an anti-cancer agent. In some embodiments, the anti-cancer agent can include a known anti-cancer agent and include, but is not limited to, one or more Abecma (Idecabtagene Vicleucel), Abemaciclib, Abiraterone Acetate, Abraxane (Paclitaxel Albumin-stabilized Nanoparticle Formulation), ABVD, ABVE, ABVE-PC, AC, Acalabrutinib Maleate Monohydrate, AC-T, Actemra (Tocilizumab), Adagrasib, Adcetris (Brentuximab Vedotin), ADE, Ado-Trastuzumab Emtansine, Adriamycin (Doxorubicin Hydrochloride), Adstiladrin (Nadofaragene Firadenovec-vncg), Afamitresgene Autoleucel, Afatinib Dimaleate, Afinitor (Everolimus), Akeega (Niraparib Tosylate Monohydrate and Abiraterone Acetate), Akynzeo (Netupitant and Palonosetron Hydrochloride), Aldara (Imiquimod), Aldesleukin, Alectinib, Alemtuzumab, Alimta (Pemetrexed Disodium), Alkeran for Injection (Melphalan Hydrochloride), Alkeran Tablets (Melphalan), Aloxi (Palonosetron Hydrochloride), Alpelisib, Alunbrig (Brigatinib), Alymsys (Bevacizumab), Ameluz (Aminolevulinic Acid Hydrochloride), Amifostine, Aminolevulinic Acid Hydrochloride, Amivantamab-vmjw, Amtagvi (Lifileucel), Anastrozole, Anktiva (Nogapendekin Alfa Inbakicept-pmln), Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asciminib Hydrochloride, Asparaginase Erwinia Chrysanthemi, Asparaginase Erwinia Chrysanthemi (Recombinant)-rywn, Asparlas (Calaspargase Pegol-mknl), Atezolizumab, Augtyro (Repotrectinib), Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Beleodaq (Belinostat), Belinostat, Belzutifan, Bendamustine Hydrochloride, Bendeka (Bendamustine Hydrochloride), BEP, Besponsa (Inotuzumab Ozogamicin), Besremi (Ropeginterferon Alfa-2b-njft), Bevacizumab, Bexarotene, Bicalutamide, BiCNU (Carmustine), Binimetinib, Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Breyanzi (Lisocabtagene Maraleucel), Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib Maleate Monohydrate), Camcevi (Leuprolide Mesylate), Campath (Alemtuzumab), Camptosar (Irinotecan Hydrochloride), Capecitabine, Capivasertib, Caplacizumab-yhdp, Capmatinib Hydrochloride, CAPOX, Carac (Fluorouracil Topical), Carboplatin, Carboplatin-Taxol, Carfilzomib, Carmustine, Carvykti (Ciltacabtagene Autoleucel), Casodex (Bicalutamide), CEM, Cemiplimab-rwlc, Ceritinib, Cervarix (Recombinant HPV Bivalent Vaccine), Cetuximab, CEV, Chlorambucil, Chlorambucil-Prednisone, CHOP, Ciltacabtagene Autoleucel, Cisplatin, Cladribine, Clofarabine, CMF, Cobimetinib Fumarate, Columvi (Glofitamab-gxbm), Cometriq (Cabozantinib-S-Malate), COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacomitinib, Dactinomycin, Danyelza (Naxitamab-gqgk), Daratumumab, Daratumumab and Hyaluronidase-fihj, Darbepoetin Alfa, Darolutamide, Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Degarelix, Denileukin Diftitox-cxdl, Denosumab, Dexamethasone, Dexrazoxane Hydrochloride, Dinutuximab, Docetaxel, Dostarlimab-gxly, Doxorubicin Hydrochloride, Doxorubicin Hydrochloride Liposome, Durvalumab, Duvelisib, Eflornithine Hydrochloride, Efudex (Fluorouracil Topical), Elacestrant Dihydrochloride, Elahere (Mirvetuximab Soravtansine-gynx), Eligard (Leuprolide Acetate), Elitek (Rasburicase), Ellence (Epirubicin Hydrochloride), Elotuzumab, Elranatamab-bcmm, Eltrombopag Olamine, Elzonris (Tagraxofusp-erzs), Emapalumab-lzsg, Emend (Aprepitant), Enasidenib Mesylate, Encorafenib, Enfortumab Vedotin-ejfv, Enhertu (Fam-Trastuzumab Deruxtecan-nxki), Entrectinib, Enzalutamide, Epcoritamab-bysp, Epirubicin Hydrochloride, Epkinly (Epcoritamab-bysp), EPOCH, Epoetin Alfa, Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Everolimus, Evista (Raloxifene Hydrochloride), Evomela (Melphalan Hydrochloride), Exemestane, 5-FU (Fluorouracil Injection), 5-FU (Fluorouracil Topical), Fam-Trastuzumab Deruxtecan-nxki, Fareston (Toremifene), Faslodex (Fulvestrant), FEC, Fedratinib Hydrochloride, Femara (Letrozole), Filgrastim, Fludarabine Phosphate, Fluorouracil Injection, Fluorouracil Topical, Flutamide, FOLFIRI, FOLFIRI-Bevacizumab, FOLFIRI-Cetuxamab, FOLFIRINOX, FOLFOX, Folotyn (Pralatrexate), Fostamatinib Disodium, Fotivda (Tivozanib Hydrochloride), Fruquintinib, Fulphila (Pegfilgrastim), FU-LV, Fulvestrant, Futibatinib, Fyarro (Sirolimus Protein-Bound articles), Gamifant (Emapalumab-lzsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, Gemcitabine-Cisplatin, Gemcitabine-Oxaliplatin, Gemtuzumab Ozogamicin, Gilotrif (Afatinib Dimaleate), Gilteritinib Fumarate, Glasdegib Maleate, Gleevec (Imatinib Mesylate), Gliadel Wafer (Carmustine Implant), Glofitamab-gxbm, Glucarpidase, Goserelin Acetate, Granisetron, Granisetron Hydrochloride, Granix (Filgrastim), Halaven (Eribulin Mesylate), Hemangeol (Propranolol Hydrochloride), Hepzato (Melphalan Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idecabtagene Vicleucel, Idelalisib, Idhifa (Enasidenib Mesylate), Ifosfamide, IL-2 (Aldesleukin), Imatinib Mesylate, Imdelltra (Tarlatamab-dlle), Imetelstat Sodium, Imfinzi (Durvalumab), Imiquimod, Imjudo (Tremelimumab-actl), Imlygic (Talimogene Laherparepvec), Infugem (Gemcitabine Hydrochloride), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Sucrosofate, Isatuximab-irfc, Istodax (Romidepsin), Ivosidenib, Iwilfin (Eflornithine Hydrochloride), Ixabepilone, Ixazomib Citrate, Jakafi (Ruxolitinib Phosphate), Jaypirca (Pirtobrutinib), JEB, Jelmyto (Mitomycin), Jemperli (Dostarlimab-gxly), Jevtana (Cabazitaxel), Kadcyla (Ado-Trastuzumab Emtansine), Kepivance (Palifermin), Keytruda (Pembrolizumab), Kimmtrak (Tebentafusp-tebn), Kisqali (Ribociclib), Koselugo (Selumetinib Sulfate), Krazati (Adagrasib), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Letrozole, Leucovorin Calcium, Leukeran (Chlorambucil), Leuprolide Acetate, Leuprolide Mesylate, Levulan Kerastick (Aminolevulinic Acid Hydrochloride), Libtayo (Cemiplimab-rwlc), Lifileucel, Lisocabtagene Maraleucel, Lomustine, Loncastuximab Tesirine-lpyl, Lonsurf (Trifluridine and Tipiracil Hydrochloride), Loqtorzi (Toripalimab-tpzi), Lorlatinib, Lumakras (Sotorasib), Lumoxiti (Moxetumomab Pasudotox-tdfk), Lunsumio (Mosunetuzumab-axgb), Lupron Depot (Leuprolide Acetate), Lurbinectedin, Luspatercept-aamt, Lutathera (Lutetium Lu 177-Dotatate), Lutetium (Lu 177-Dotatate), Lutetium Lu 177 Vipivotide Tetraxetan, Lymphir (Denileukin Diftitox-cxdl), Lynparza (Olaparib), Lytgobi (Futibatinib), Margenza (Margetuximab-cmkb), Margetuximab-cmkb, Matulane (Procarbazine Hydrochloride), Mechlorethamine Hydrochloride, Megestrol Acetate, Mekinist (Trametinib Dimethyl Sulfoxide), Mektovi (Binimetinib), Melphalan, Melphalan Hydrochloride, Mercaptopurine, Mesna, Methotrexate Sodium, Methylnaltrexone Bromide, Midostaurin, Mirvetuximab Soravtansine-gynx, Mitomycin, Mitoxantrone Hydrochloride, Mogamulizumab-kpkc, Momelotinib Dihydrochloride Monohydrate, Monjuvi (Tafasitamab-cxix), MOPP, Mosunetuzumab-axgb, Moxetumomab Pasudotox-tdfk, Mozobil (Plerixafor), MVAC, Mvasi (Bevacizumab), Myleran (Busulfan), Mylotarg (Gemtuzumab Ozogamicin), Nadofaragene Firadenovec-vncg, Nanoparticle Paclitaxel (Paclitaxel Albumin-stabilized Nanoparticle Formulation), Naxitamab-gqgk, Necitumumab, Nelarabine, Neratinib Maleate, Netupitant and Palonosetron Hydrochloride, Neulasta (Pegfilgrastim), Neupogen (Filgrastim), Nexavar (Sorafenib Tosylate), Nilotinib, Nilutamide, Ninlaro (Ixazomib Citrate), Niraparib Tosylate Monohydrate, Niraparib Tosylate Monohydrate and Abiraterone Acetate, Nirogacestat Hydrobromide, Nivestym (Filgrastim), Nivolumab, Nivolumab and Relatlimab-rmbw, Nogapendekin Alfa Inbakicept-pmln, Nplate (Romiplostim), Nubeqa (Darolutamide), Nyvepria (Pegfilgrastim), Obinutuzumab, Odomzo (Sonidegib), OEPA, Ofatumumab, OFF, Ogsiveo (Nirogacestat Hydrobromide), Ojemda (Tovorafenib), Ojjaara (Momelotinib Dihydrochloride Monohydrate), Olaparib, Olutasidenib, Omacetaxine Mepesuccinate, Oncaspar (Pegaspargase), Ondansetron Hydrochloride, Onivyde (Irinotecan Sucrosofate), Onureg (Azacitidine), Opdivo (Nivolumab), Opdualag (Nivolumab and Relatlimab-rmbw), OPPA, Orgovyx (Relugolix), Orserdu (Elacestrant Dihydrochloride), Osimertinib Mesylate, Oxaliplatin, Paclitaxel, Paclitaxel Albumin-stabilized Nanoparticle Formulation, Pacritinib Citrate, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Paraplatin (Carboplatin), Pazopanib Hydrochloride, PCV, PEB, Pegaspargase, Pegfilgrastim, Pemazyre (Pemigatinib), Pembrolizumab, Pemetrexed Disodium, Pemigatinib, Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Pirtobrutinib, Plerixafor, Pluvicto (Lutetium Lu 177 Vipivotide Tetraxetan), Polatuzumab Vedotin-piiq, Pomalidomide, Pomalyst (Pomalidomide), Ponatinib Hydrochloride, Portrazza (Necitumumab), Poteligeo (Mogamulizumab-kpkc), Pralatrexate, Pralsetinib, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Qinlock (Ripretinib), Quizartinib Dihydrochloride, Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Relugolix, R-EPOCH, Repotrectinib, Retacrit (Epoetin Alfa), Retevmo (Selpercatinib), Retifanlimab-dlwr, Rezlidhia (Olutasidenib), Riabni (Rituximab), Ribociclib, R-ICE, Ripretinib, Rituximab, Rituximab and Hyaluronidase Human, Rolapitant Hydrochloride, Romidepsin, Romiplostim, Ropeginterferon Alfa-2b-njft, Rozlytrek (Entrectinib), Rucaparib Camsylate, Ruxolitinib Phosphate, Rybrevant (Amivantamab-vmjw), Rydapt (Midostaurin), Rylaze (Asparaginase Erwinia Chrysanthemi [Recombinant]-rywn), Rytelo (Imetelstat Sodium), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Scemblix (Asciminib Hydrochloride), Siltuximab, Sipuleucel-T, Sirolimus Protein-Bound Particles, Soltamox (Tamoxifen Citrate), Somatuline Depot (Lanreotide Acetate), Sonidegib, Sorafenib Tosylate, Sotorasib, Sprycel (Dasatinib), STANFORD V, Sterile Talc Powder (Talc), Stivarga (Regorafenib), Sunitinib Malate, Sustol (Granisetron), Sutent (Sunitinib Malate), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talimogene Laherparepvec, Talquetamab-tgvs, Talvey (Talquetamab-tgvs), Talzenna (Talazoparib Tosylate), Tamoxifen Citrate, Tarceva (Erlotinib Hydrochloride), Targretin (Bexarotene), Tarlatamab-dlle, Tasigna (Nilotinib), Tavalisse (Fostamatinib Disodium), Taxotere (Docetaxel), Tazemetostat Hydrobromide, Tazverik (Tazemetostat Hydrobromide), Tebentafusp-tebn, Tecartus (Brexucabtagene Autoleucel), Tecelra (Afamitresgene Autoleucel), Tecentriq (Atezolizumab), Teclistamab-cqyv, Temozolomide, Temsirolimus, Tepmetko (Tepotinib Hydrochloride), Tepotinib Hydrochloride, Tevimbra (Tislelizumab-jsgr), Thalidomide, Thioguanine, Thiotepa, Tibsovo (Ivosidenib), Tisagenlecleucel, Tislelizumab-jsgr, Tisotumab Vedotin-tftv, Tivdak (Tisotumab Vedotin-tftv), Tivozanib Hydrochloride, Tocilizumab, Tolak (Fluorouracil Topical), Topotecan Hydrochloride, Toremifene, Toripalimab-tpzi, Torisel (Temsirolimus), Totect (Dexrazoxane Hydrochloride), Tovorafenib, TPF, Trabectedin, Trametinib Dimethyl Sulfoxide, Trastuzumab, Trastuzumab and Hyaluronidase-oysk, Treanda (Bendamustine Hydrochloride), Tremelimumab-actl, Trexall (Methotrexate Sodium), Trifluridine and Tipiracil Hydrochloride, Trisenox (Arsenic Trioxide), Trodelvy (Sacituzumab Govitecan-hziy), Truqap (Capivasertib), Truxima (Rituximab), Tucatinib, Turalio (Pexidartinib Hydrochloride), Tykerb (Lapatinib Ditosylate), Ultomiris (Ravulizumab-cwvz), Undencyca (Pegfilgrastim), Unituxin (Dinutuximab), Uridine Triacetate, VAC, Valrubicin, VAMP, Vandetanib, Vanflyta (Quizartinib Dihydrochloride), Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vinblastine Sulfate, Vincristine Sulfate, Vinorelbine Tartrate, VIP, Vismodegib, Vistogard (Uridine Triacetate), Vitrakvi (Larotrectinib Sulfate), Vizimpro (Dacomitinib), Vonjo (Pacritinib Citrate), Voranigo (Vorasidenib Citrate), Vorasidenib Citrate, Voraxaze (Glucarpidase), Vorinostat, Votrient (Pazopanib Hydrochloride), Vyxeos (Daunorubicin Hydrochloride and Cytarabine Liposome), Welireg (Belzutifan), Xalkori (Crizotinib), Xatmep (Methotrexate Sodium), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (Ibritumomab Tiuxetan), Ziextenzo (Pegfilgrastim), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), Zynlonta (Loncastuximab Tesirine-lpyl), Zynyz (Retifanlimab-dlwr), Zytiga (Abiraterone Acetate), experimental agent or combinations thereof. In other embodiments, the anti-cancer agent can include an experimental anti-cancer agent.

In some embodiments, the therapeutic agent can be an immunomodulatory agent of which the humanized mouse can be tested for efficacy and at least one tissue from a patient having an immunomodulatory disorder has been engrafted onto or delivered to, the humanized mouse. In accordance with these embodiments, the immunomodulatory agent can be an experimental or known immunomodulatory agent; for example, one or more of Pembrolizumab, Nivolumab, Cemiplima, Dostarlimab, Retifanlimab, Atezolizumab, Avelumab, Durvalumab, Ipilimumab, Tremelimumab, and the like, or combinations thereof.

In some embodiments, it is understood that the disclosed humanized mouse contains T-cell lineages within the model system representative of that found in a corresponding patient. In accordance with these embodiments, a basis of the clinical efficacy of at least one immune checkpoint inhibitors (ICIs) treatment or other anti-cancer, immunomodulating agent can be effectively studied. For example, in certain embodiments, to evaluate if patient-derived sTOs as disclosed herein used to create a patient-specific humanize mouse can support T-cell development and education, studies can be performed to characterize sTO capabilities both in vitro and in vivo, as disclosed herein. In certain embodiments, these studies can include, but are not limited to, whole cell assessments (e.g., determining protein expression or cell viability using any method known in the art, for example, flow cytometry), protein presence, absence and/or expression (e.g., by Western blot analysis, immunoassay such as ELISA and the like, or intracellular staining), nucleic acid polynucleotide levels or presence, modification, and/or expression (e.g., DNA sequencing, RNA sequencing, Southern blot, Northern blot, and genome assays), lipid analysis, and/or sugar analysis.

In some embodiments, an autologous humanized mouse is disclosed where the autologous PDX HM can be generated from HSPCs isolated after G-CSF mobilization in a patient who subsequently underwent a biopsy to establish or obtain PDX tissues. In these examples, such a mouse serves as a reproducible platform in which patient immune cells and tumors can coexist and contains components needed to recreate a clinical cancer tumor microenvironment (TME).

In certain embodiments, it is recognized that although several PDX HM mice have previously been described in which T-cell education occurs in a murine thymus or in implanted fetal thymic tissue, these T-cells are exclusively educated in a xenogeneic or allogeneic environment, differing from the currently disclosed model. For example, in an HM model which pairs surgically excised neonatal thymus with HSCs isolated umbilical cord blood, the T-cells educated in this context are allogeneic to implanted PDX tissue. It has not been previously feasible to procure patient thymic tissue to incorporate into PDX HM. Embodiments disclosed herein demonstrate that patient PBMCs can be reprogrammed to iPSCs in vitro and these cells can then be differentiated into TECs in the form of sTOs as previously described, which can then be implanted into a humanized mouse, where they direct T-cell education in an environment autologous to that of the patient in a humanized, personalized setting, specific to the patient of which they were obtained. Such a model represents a significant step in the ex vivo/in vivo production of patient-derived thymic tissue in culture. TECs found within sTOs derived by these methods express genes critical for negative selection and can facilitate the development of pro-T-cells and double positive (DP) and/or single positive T-cells from allogenic and HLA matched human cord blood. The methods described herein supports that PBMCs can be used to generate autologous thymic tissue (sTOs) matched to patient tissue (e.g., a patient-derived tumor), and that the humanized mouse and methods reported herein possess significant potential for assessing therapeutic interventions without the need to test a patient with a multitude of agents that may or may not be effective. In accordance with these embodiments, the patient-specific humanized mouse disclosed herein can be used; for example, to test a few or a multitude of therapeutic agents to treat the modeled condition of the patient and provide an educated therapeutic agent approach to treating the patient. One, or more therapeutic agents can be provided to humanized mice engrafted with tissue from a patient suffering one or more of the conditions contemplated herein to determine the efficacy of the one or more therapeutic agents.

In certain embodiments, HSPCs obtained from a humanized mouse engrafted with HLA matched cord-blood derived HSPCs can be used, where this semi-matched environment demonstrated that T-cell education produced active T-cells more similar to those found in the patient than previously observed models. In accordance with these embodiments, a range of T cell production and infiltration in the mice was found to have successfully engrafted sTOs. In certain embodiments, reasons for successful implantation can be due at least in part to sTO tissue implanted in an area with sufficient capsular vasculature for a blood supply. In other embodiments, a baseline degree of mouse humanization can provide adequate pro-T-cells for sTO-derived thymic activity. In accordance with these embodiments, a baseline degree of humanization can include where the humanized mouse can include at least about 0.1% human tissue, at least about 0.2% human tissue, at least about 0.3% human tissue, at least about 0.4% human tissue, at least about 0.5% human tissue, at least about 0.6% human tissue, at least about 0.7% human tissue, at least about 0.8% human tissue, at least about 0.9% human tissue, at least about 1.0% human tissue, at least about 1.5% human tissue, at least about 2.0% human tissue, at least about 2.5% human tissue, at least about 3.0% human tissue, at least about 3.5% human tissue, at least about 4.0% human tissue, at least about 4.5% human tissue, at least about 5.0% human tissue, at least about 5.5% human tissue, at least about 6.0% human tissue, at least about 6.5% human tissue, at least about 7.0% human tissue, at least about 7.5% human tissue, at least about 8.0% human tissue, at least about 8.5% human tissue, at least about 9.0% human tissue, at least about 9.5% human tissue, at least about 10.0% human tissue, by mass, or more. In addition, some degree of transient T cell education can have occurred but had ceased when the sTO tissues were harvested, because T cells were observed in the peripheral blood and spleens of the mHM_{TA} in which sTO engraftment was documented. Notably, the mHM_{TA} with highest T-cell production had a higher percentage of human CD45⁺ cells in its bone marrow (e.g., 71.1%) than certain counterparts. Interestingly, T-cells educated in a murine thymus occurred in mHM animals with comparably low levels of human CD45⁺ bone marrow cells, so education in the sTO thymic environment can be more demanding. In certain embodiments, from a clinical perspective tumor-invading T-cells produced in the mHM_{TA} model demonstrated an increased capacity to reduce tumor growth. Single cell sequencing of circulating T-cells in conjunction with exome profiling of the PDX tumor tissue suggests that mHM models can be more permissive to tumor growth due to an increased abundance of Tregs. In addition, exome profiling of the PDX tumors demonstrated a lower abundance of subclonal mutations in the mHM_{TA} model, which is consistent with effective immunoediting in the less permissive, CD8⁺ TEM-enriched mHM_{TA} system.

In other embodiments, intratumoral heterogeneity is well known and can be represented in exome sequencing data and further can be used to monitor tumor dynamics (e.g., a change in tumor size or tumor composition, reduced metastasis, a change in tumor-infiltrating T cell populations, including the number or ratio of CD4⁺ and/or CD8⁺ T cells for example is elevated, and/or a change in tumor genetic makeup) in response to a given therapeutic treatment. In other embodiments, by assessing genomic rather than transcriptomic changes, changes reported can be a result of stable clonal selection and not transient changes in gene expression. For example, using HLA binding predictions, putative neoantigens can be analyzed for change comparing mHM and mHM_{TA} tumors can be compared.

In other embodiments, it has been demonstrated herein that patient-autologous T-cell education in a mouse humanized with a patient-derived xenograft (PDX HM) is possible and produces active T-cells representative of those found in the patient of which the humanized mouse was designed. Embodiments disclosed herein demonstrate that these T-cells in these models can slow tumor growth. These observations bridge the gap between the previously described autologous HM model and the instantly described humanized mouse employing sTOs generated from human PBMCs as disclosed herein. In accordance with these embodiments and further to the methods and compositions of use provided herein validation that a completely autologous sTO-containing PDX HM has been created and will be sought-after of use to further analyze efficacy of therapeutic intervention of health conditions disclosed herein in patient-specific manners for improved outcome.

Other embodiments of the present disclosure relate to generation and use of sTOs in a humanized mouse to generate a patient-specific humanized mouse model. In certain embodiments, novel and efficient compositions, and methods for generating and using patient-derived thymic cells (e.g., TEP cells) from iPSCs are disclosed herein where functional thymic cells generate conventional T cells. In certain embodiments, the method for converting PBMCs to iPSCs and differentiating iPSCs to TECs and intermediary cells (e.g., DE, AFE, VPE cells) to prepare sTOs can include compositions comprising a culturing media. In some embodiments, in vitro preparation of sTOs from PBMCs can be carried out in compositions which can include a base medium that contains one or more additives as disclosed herein. In certain embodiments, compositions disclosed herein can be used in one or more stages of differentiation for producing thymic cells from PBMCs via iPSCs.

In some embodiments, in a patient specific manner, susceptibility of a patient's immune system to being activated by a variety of checkpoint inhibitors can be assessed using the humanized mouse disclosed herein. Further to these embodiments, sTOs generated from a patient's peripheral blood cells generate and/or educate the patient T cells, that are subsequently exposed to checkpoint inhibitors, and then degree of T cell activation can be determined in order to choose therapeutic agents that induce the patient's T cells. In accordance with these embodiments, the degree of response of a patient to a particular therapeutic agent often does not hinge upon tumor specific features, but rather immune system-specific characteristics. In certain embodiments, cancer cells or tissues from the patient can be introduced to the humanized mouse (in addition to sTOs) to investigate cancer driven factors.

Generation of sTOs
Stage 1—Culturing iPSCs to Generate Anterior Primitive Streak (APS) Cells

In certain embodiments, the present disclosure provides compositions and methods for use in vitro differentiation of PSCs into thymic cells and intermediary cells thereof. In some embodiments, PSCs used in present disclosure can include, but are not limited to, embryonic stem cells, embryonic germ cells, or iPSCs. In some embodiments, PSCs can be mammalian PSCs. In other embodiments, PSCs can be human PSCs. In other embodiments, PSCs used in present disclosure can be isolated from an autologous source. As used herein, the term “autologous” refers to obtaining PSCs from the same subject to be treated and/or monitored with the thymic cells generated as disclosed herein. In some embodiments, PSCs of use in embodiments of the present disclosure can be isolated from an allogeneic source. As used herein, the term “allogeneic” refers to obtaining PSCs from a different subject than the subject to be treated and/or monitored with thymic cells generated as disclosed herein. In some embodiments, PSCs used in present disclosure can be harvested, generated, cultured, and/or characterized using standard methods in the art.

In certain embodiments, compositions disclosed herein can be used to provide conditions for culturing PSCs to generate APS cells according to methods of the present disclosure. In accordance with these embodiments, compositions disclosed herein can include a base medium having a low concentration of protein. In certain embodiments, a protein used in a base medium as disclosed herein can be serum or other protein supplement. In some embodiments, a protein used in a base medium as disclosed herein can be serum from a mammalian source (e.g., bovine, human) or protein derived from any source.

In some embodiments, compositions disclosed herein for culturing PSCs to generate APS cells according to methods of the present disclosure can be a base medium with low protein concentrations of about 0.5% (w/v) or less. In some embodiments, a base medium with low protein concentrations for use herein can have protein concentrations of less than about 0.01% (w/v), less than about 0.05% (w/v), less than about 0.1% (w/v), less than about 0.2% (w/v), less than about 0.3% (w/v), less than about 0.4% (w/v) or less than about 0.5% (w/v).

In some embodiments, compositions disclosed herein for culturing PSCs to generate APS cells according to methods of the present disclosure can include a base medium with at least one additive capable of modulating Wnt and/or a Wnt signaling pathway. As used herein, the term “modulating” means activating, propagating, inhibiting, upregulating the expression of, downregulating the expression of, and/or otherwise modifying the activity of a signaling pathway or a component of a signaling pathway. In accordance with these embodiments, compositions disclosed herein for culturing PSCs to generate APS cells according to methods of the present disclosure can be a base medium with at least one Wnt modulating agent. As used herein, “a Wnt modulating agent” can refer to any chemical (e.g., small molecule or other chemical agent), compound, polypeptide, protein, or fragment thereof, polynucleotide (DNA or RNA), or other agent that modulates Wnt and/or a Wnt signaling pathway. In some embodiments, compositions disclosed herein for culturing PSCs to generate anterior primitive streak (APS) cells according to methods of the present disclosure can be a base medium with at least one Wnt modulating agent capable of activating Wnt and/or a Wnt signaling pathway (e.g., “a Wnt activator”). In certain embodiments, an agent capable of modulating Wnt can be a Wnt activating agent. Non-limiting examples of Wnt activators suitable for uses disclosed herein can include DNA encoding β-catenin (e.g., naked DNA encoding β-catenin, plasmid expression vectors encoding β-catenin, viral expression vectors encoding β-catenin), β-catenin polypeptides, one or more Wnt/β-catenin pathway agonists (e.g., Wnt ligands, DSH/DVL-1, -2, -3, LRP6N, WNT3A, WNT5A, and WNT3A, 5A), one or more glycogen synthase kinase 3 β (GSK3β inhibitors (e.g., lithium chloride (LiCl), Purvalanol A, olomoucine, alsterpaullone, kenpaullone, benzyl-2-methyl-1,2,4-thiadiazolidine-3,5-dione (TDZD-8), 2-thio (3-iodobenzyl)-5-(1-pyridyl)-[1,3,4]-oxadiazole (GSK3 inhibitor II), 2,4-dibenzyl-5-oxothiadiazolidine-3-thione (OTDZT), (2′Z,3′E)-6-Bromoindirubin-3′-oxime (BIO), α-4-Dibromoacetophenone (i.e., Tau Protein Kinase I (TPK I) Inhibitor), 2-Chloro-1-(4,5-dibromo-thiophen-2-yl)-ethanone, N-(4-Methoxybenzyl)-N′-(5-nitro-1,3-thiazol-2-yl) urea (AR-A014418), indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin, 5-bromoindirubin, N-(4-Methoxybenzyl)-N′-(5-nitro-1,3-thiazol-2-Aurea (AR-A014418), H-KEAPPAPPQSpP-NH2 (L803; SEQ ID NO: 18) and Myr-N-GKEAPPAPPQSpPNH2 (L803-mts; SEQ ID NO: 19)), one or more anti-sense RNA or siRNA that bind specifically to GSK3B mRNA, one or more casein kinase 1 (CK1) inhibitors (e.g., antisense RNA or siRNA that binds specifically to CK1 mRNA), and the like. In accordance with these embodiments, the Wnt modulating agent can be used at concentrations known in the art. In certain embodiments, Wnt can be provided at about 15 ng/ml to about 50 ng/ml (e.g., 25 ng/ml). In certain embodiments, CHIR can be provided at about 0.1 μM to about 10 μM (e.g., 3 μM).

In some embodiments, compositions disclosed herein for culturing iPSCs to generate APS cells according to methods of the present disclosure can be a base medium with at least one Wnt activator. In some embodiments, compositions disclosed herein for culturing PSCs to generate APS cells according to methods of the present disclosure can be a base medium with at least one Wnt activator. In some embodiments, compositions disclosed herein for culturing PSCs to generate APS cells according to methods of the present disclosure can be made up of a base medium with at least one Wnt activator.

In some embodiments, compositions disclosed herein for culturing PSCs to generate APS cells according to methods of the present disclosure can be a base medium with at least one Activin A modulating agent (and optionally, at least one Wnt activator and/or at least one BMP inhibitor). As used herein, “an Activin A modulating agent” can refer to any chemical (e.g., small molecule or other chemical agent), compound, polypeptide, protein, or fragment thereof, polynucleotide (DNA or RNA), or other agent that modulates Activin A and/or an Activin A signaling pathway. In some embodiments, compositions disclosed herein for culturing PSCs to generate APS cells according to methods disclosed herein can include a base medium with at least one Activin A activator. In accordance with these embodiments, an Activin A activator can be Activin A, and/or variants thereof or functional analogs thereof. Non-limiting examples of Activin A analogs suitable for use disclosed herein can include IDE1 (2-[6-carboxy-hexanoyl)-hydrazonomethyl]-benzoic acid), IDE2 (7-(2-cyclopentylidenehydrazino)-7-oxoheptanoic acid, and the like. In some embodiments, compositions disclosed herein can have at least one Activin A activator that can include at least one of recombinant mammalian Activin A, SB4, Alantolactone, or a combination thereof. In some embodiments, compositions disclosed herein include Activin A modulating agent which can be used at concentrations known in the art. In certain embodiments, Activin A can be provided at about 100 ng/mL to about 200 ng/mL.

In some embodiments, compositions disclosed herein for culturing PSCs to generate APS cells according to methods of the present disclosure can include a base medium with at least one Activin A activator (and optionally, at least one Wnt activator and/or at least one BMP inhibitor). In some embodiments, compositions disclosed herein for culturing PSCs to generate APS cells according to methods of the present disclosure can be a base medium with at least one Activin A activator (and optionally, at least one Wnt activator and/or at least one BMP inhibitor). In some embodiments, compositions disclosed herein for culturing PSCs to generate APS cells according to methods of the present disclosure can include a base medium with at least one Activin A activator (and optionally, at least one Wnt activator and/or at least one BMP inhibitor). In certain embodiments, the base media is a low protein-containing (e.g., 1% (w/v) protein or less) media.

In some embodiments, compositions for culturing PSCs to generate APS cells can be a base medium with at least one phosphoinositide 3-kinase (PI3K) modulating agent (and optionally, at least one Activin A inhibitor, at least one Wnt activator and/or at least one BMP inhibitor). As used herein, “a PI3K modulating agent” can refer to any chemical (e.g., small molecule or other chemical agent), compound, polypeptide, protein, or fragment thereof, polynucleotide (DNA or RNA), or other agent that modulates PI3K and/or a PI3K signaling pathway. In some embodiments, compositions disclosed herein for culturing PSCs to generate APS cells according to methods of the present disclosure can be a base medium with at least one PI3K inhibitor. Non-limiting examples of PI3K inhibitors suitable for use in compositions and methods disclosed herein can include BKM120, BEX235, BGT226, Idelalisib, GDC-0941, IPI-145 (INK1197), GSK2636771, PI-103, BEZ235, BGT226, VS-5584m, (SB2343), PI-103, ZSTK474, GSK1059615, Gedatolisib, HS-173, Alpelisib (BYL719), PIK-75, A66, YM201636, TGX-221, GSK2636771, CZC24832, AS-252424, AS-604850, CAY10505, CAL-101 (Idelalisib, GS-1101), PIK-294, PI-3065, PIK-293, IC-87114, AS-605240, PIK-90, other PI3K inhibitors and the like and combinations thereof. In some embodiments, compositions disclosed herein PI3K modulating agent can be used at concentrations known in the art. In certain embodiments, a PI3K modulating can be provided at about 0.1 μM to about 10 μM (e.g., about 1 μM).

In certain embodiments, compositions for culturing PSCs to generate APS cells can be a base medium with a low concentration of protein (e.g., 0.5% (w/v) protein or less) and at least one of: at least one Wnt modulating agent, at least one Activin A modulating agent, at least one PI3K modulating agent, or a combination thereof. In some embodiments, compositions disclosed herein for culturing PSCs to generate APS cells according to methods disclosed herein can include a base medium with a low concentration of protein and at least one of: at least one Wnt activator, at least one Activin A activator, at least one PI3K inhibitor, or a combination thereof. In some embodiments, compositions disclosed herein for culturing PSCs to generate APS cells according to methods of the present disclosure can be a base medium with a low concentration of protein, and at least one Wnt activator, at least one Activin A activator, and at least one PI3K inhibitor and optionally other culturing agents.

In certain embodiments, compositions disclosed herein for culturing PSCs to generate APS cells can be provided to the cell culture and incubated for a period of time suitable for PSCs cells to differentiate into APS cells. In some embodiments, culture condition disclosed herein can be from about 12 hours to about 48 hours or to about 72 hours or more. In some embodiments, compositions disclosed herein for culturing PSCs to generate APS cells can be provided to the cell culture and incubated for a period of time suitable for about 70%, about 75%, about 80%, about 85%, about 90%, about 95%, or about 99% of the PSCs cells to differentiate into APS cells. In some embodiments, compositions disclosed herein for culturing PSCs to generate APS cells can be provided to the cell culture and incubated for a period of about 12 hours to about 5 days.

### Stage 2—Culturing APS Cells to Generate Definitive Endoderm (DE) Cells

In certain embodiments, compositions disclosed herein can be used to provide conditions for culturing APS cells to generate DE cells according to methods of the present disclosure. In accordance with these embodiments, compositions disclosed herein can be a base medium with a low concentration of protein (e.g., a “low-protein medium”) or a high concentration of protein (e.g., a “protein enriched medium”). In some embodiments, compositions disclosed herein can be used to provide conditions for culturing APS cells to generate DE cells according to methods of the present disclosure can be a base medium with high protein concentrations of about 0.5% (w/v) or more protein up to about 30% (w/v) protein (e.g., about 0.5% to about 20.0% (w/v)) protein. In some embodiments, a base medium with high protein concentrations of use as culturing media disclosed herein can have protein concentrations of more than about 0.5% (w/v), more than about 5.0% (w/v), more than about 10% (w/v), more than about 15% (w/v), more than about 20% (w/v), more than about 25% (w/v), up to about 30.0% (w/v).

In certain embodiments, compositions disclosed herein for culturing APS cells to generate DE cells according to methods of the present disclosure can include a base medium with at least one bone morphogenetic protein (BMP) modulating agent. As used herein, “a BMP modulating agent” can refer to any chemical (e.g., small molecule or other chemical agent), compound, polypeptide, protein, or fragment thereof, polynucleotide (DNA or RNA), or other agent that modulates BMP and/or a BMP signaling pathway. In some embodiments, compositions disclosed herein for culturing APS cells to generate DE cells according to methods of the present disclosure can be a base medium with at least one BMP inhibitor. Non-limiting examples of BMP inhibitors suitable for uses and supplements in enriched protein media disclosed herein can include, but is not limited to, LDN193189, LDN214117, LDN212854, DMH2, K02288, ML347, SGC AAK1 1, PD407824, UK383367, and A01, recombinant mammalian Noggin and Dorsomorphin, non-mammalian Noggin and Dorsomorphin, and the like, or any combination thereof. Any BMP inhibitor is contemplated of use herein to reduce or inhibit the BMP pathway. In some embodiments, compositions disclosed herein for culturing APS cells to generate DE cells according to methods of the present disclosure can be a base medium with at least one BMP inhibitor. In some embodiments, compositions disclosed herein for culturing APS cells to generate DE cells according to methods of the present disclosure can be a base medium with at least one BMP inhibitor at a concentration of at least about 5 ng/ml to at least about 1000 ng/mL. In some embodiments, compositions disclosed herein for culturing APS cells to generate DE cells according to methods of the present disclosure can be a base medium having a high protein concentrations of at least 1.0% (w/v) or more and further include at least one BMP inhibitor at a concentration of about 5 ng/mL to about 350 ng/ml (e.g., about 10 ng/mL to about 150 ng/mL, about 15 ng/mL to about 100 ng/mL, or about 15 ng/ml to about 50 ng/mL).

In certain embodiments, compositions disclosed herein for culturing APS cells to generate DE cells according to methods of the present disclosure can include a base medium with at least one Activin A modulating agent (and at least one BMP modulator). In some embodiments, compositions disclosed herein for culturing APS cells to generate DE cells according to methods of the present disclosure can include a base medium with at least one Activin A inhibitor (and at least one BMP modulator) and, optionally, include low protein concentration levels as disclosed herein. Non-limiting examples of Activin A inhibitors include A83-01, RepSox, D4476, Ly364947, R268712, SD208, SB505124, SM16, Galunisertib, SB525334, and SB431542, and the like. In some embodiments, compositions disclosed herein for culturing APS cells to generate DE cells according to methods of the present disclosure can include a base medium with at least one Activin A inhibitor (and at least one BMP modulator) at a concentration of at least about 5 ng/mL to at least about 2 μg/mL, for example.

In certain embodiments, compositions disclosed herein for culturing APS cells to generate DE cells according to methods of the present disclosure can be a base medium with an enriched concentration of protein as disclosed herein and at least one BMP modulating agent, at least one Activin A modulating agent, or a combination thereof. In some embodiments, compositions disclosed herein for culturing APS cells to generate DE cells according to methods of the present disclosure can include a base medium with an enriched concentration of protein and at least one BMP inhibitor, at least one Activin A inhibitor, or a combination thereof. In some embodiments, compositions disclosed herein for culturing APS cells to generate DE cells according to methods of the present disclosure can include a base medium with an enriched concentration of protein, at least one BMP inhibitor, and at least one Activin A inhibitor.

In some embodiments, compositions disclosed herein for culturing APS cells to generate DE cells according to methods of the present disclosure can include a base medium with at least one Activin A activator. In some embodiments, compositions disclosed herein for culturing APS cells to generate DE cells according to methods of the present disclosure can be a base medium with at least one Activin A activator at a concentration of at least about 5 ng/ml to about 1000 ng/ml, for example.

In certain embodiments, compositions disclosed herein for culturing APS cells to generate DE cells can be provided to the cell culture after the previous culture medium is removed or washed away, as appropriate. In accordance with these embodiments, compositions disclosed herein for culturing APS cells to generate DE cells can be provided to an APS cell culture after the previous culture medium is removed with or without a wash step before introduction of the fresh media.

In certain embodiments, compositions disclosed herein for culturing APS cells to generate DE cells can be provided to an APS cell culture and incubated for a period of time suitable for APS cells to differentiate into DE cells. In some embodiments, compositions disclosed herein for culturing APS cells to generate DE cells can be provided to an APS cell culture and incubated for a period of time suitable for at least about 70% to at least about 99% or 100% of the APS cells to differentiate into DE cells. In some embodiments, compositions disclosed herein for culturing APS cells to generate DE cells can be provided to an APS cell culture and incubated for a period of about 12 hours to about 5 days.

Stages 3 and 4—Culturing DE Cells to Generate Anterior Foregut Endoderm (AFE) Cells and Culturing AFE Cells to Generate Ventral Pouch Endoderm (VPE) Cells or Culturing DE Cells to Generate Pharyngeal Endoderm (PE) Cells and Culturing PE Cells to Generate VPE Cells

In certain embodiments, compositions disclosed herein can be used to provide conditions for culturing DE cells to generate PE and/or AFE cells and/or for culturing PE and/or AFE cells to generate VPE cells according to methods of the present disclosure. In accordance with these embodiments, compositions disclosed herein can include a base medium with a high concentration of protein (e.g., a “protein rich medium”). In some embodiments, compositions disclosed herein can be used to provide conditions for culturing DE cells to generate AFE cells and/or for culturing AFE cells to generate VPE cells according to methods of the present disclosure can include a base medium with a high protein concentrations of at least about 1.0% (w/v) or more, up to about 30% (w/v). In some embodiments, a base medium with high protein concentrations for use herein can have a protein concentration ranging from at least about 1.0% (w/v) to about 30% (w/v) protein. In accordance with these embodiments, protein of use to supplement medias disclosed herein can be derived from any source. In certain embodiments, the protein can be supplemented using serum, for example, human serum albumin (HSA), bovine serum albumin (BSA), fetal bovine serum (FBS), or other serum or other protein source used in culture medias.

In certain embodiments, compositions disclosed herein for culturing DE cells to generate AFE cells and/or for culturing AFE cells to generate VPE cells according to methods of the present disclosure can include a base medium with at least one BMP modulating agent. In some embodiments, compositions disclosed herein for culturing DE cells to generate AFE cells and/or for culturing AFE cells to generate VPE cells according to methods of the present disclosure can include a base medium with at least one BMP activator. In certain embodiments, these culturing compositions do not include a BMP pathway inhibiting agent. Non-limiting examples of BMP activators include, but are not limited to, BMP4, SB4, and the like. In some embodiments, compositions disclosed herein for culturing DE cells to generate AFE cells and/or for culturing AFE cells to generate VPE cells according to methods of the present disclosure can include a protein enriched media of at least about 0.5% % (w/v) protein or more. In some embodiments, a base medium with high protein concentrations for use herein can have protein concentrations of at least about 0.5% (w/v) or more, up to about 30% (w/v) and at least one BMP activator. The BMP activator concentration can be at least about 5 ng/mL to about 1000 ng/mL. In some embodiments, compositions disclosed herein for culturing DE cells to generate AFE cells and/or for culturing AFE cells to generate VPE.

In certain embodiments, compositions disclosed herein for culturing DE cells to generate AFE cells and/or for culturing AFE cells to generate VPE cells according to methods of the present disclosure can include a media with an enriched protein concentrations of at least 0.5% (w/v) to about 20% (w/v) and at least one fibroblast growth factor receptor (FGFR) activating agent (and, optionally, at least one BMP activating agent). As used herein, “a FGFR activating agent” can refer to any chemical (e.g., small molecule or other chemical agent), compound, polypeptide, protein, or fragment thereof, polynucleotide (DNA or RNA), or other agent that activates an FGFR and/or an FGFR signaling pathway. In some embodiments, compositions disclosed herein for culturing DE cells to generate AFE cells and/or for culturing AFE cells to generate VPE cells according to methods of the present disclosure can be a media with enriched protein concentrations of at least 0.5% (w/v) and at least one FGFR activator (an at least one BMP activator). In some embodiments, compositions disclosed herein for culturing DE cells to generate AFE cells and/or for culturing AFE cells to generate VPE cells according to methods of the present disclosure can be a base medium with at least one FGFR3 activator. Non-limiting examples of FGFR3 activators suitable for use herein can include a fibroblast growth factor (FGF), such as FGF8, FGF1, FGF2, FGF9, or a variant thereof, Heparin, and the like.

In some embodiments, compositions disclosed herein for culturing DE cells to generate AFE cells and/or for culturing AFE cells to generate VPE cells according to methods of the present disclosure can be a media with a high protein concentration of at least 0.5% (w/v) and at least one FGFR3 activator (e.g., FGF8, FGF1, FGF2, FGF9, or a variant thereof.

In certain embodiments, compositions disclosed herein for culturing DE cells to generate AFE cells and/or for culturing AFE cells to generate VPE cells according to methods of the present disclosure can include a media having an enriched protein concentration of at least 0.5% (w/v) protein and at least one retinoic acid signaling modulating agent (and optionally, heparin, at least one BMP activator, and/or at least one FGFR3 activator). As used herein, “a retinoic acid signaling modulating agent” can refer to any chemical (e.g., small molecule or other chemical agent), compound, polypeptide, protein, or fragment thereof, polynucleotide (DNA or RNA), or other agent that activates retinoic acid, a retinoic acid receptor, and/or a retinoic acid signaling pathway. In some embodiments, compositions disclosed herein for culturing DE cells to generate AFE cells and/or for culturing AFE cells to generate VPE cells according to methods of the present disclosure can include a media having a high protein concentration of at least 0.5% (w/v) protein and at least one Retinoic Acid signaling activating agent (and optionally, heparin, at least one BMP activator, and/or at least one FGFR3 activator). Non-limiting examples of retinoic acid signaling activators include, but are not limited to, Vitamin A, TTNPB, AC261066, SR1078, SR221, BMS493, Fenretinide, AM580, Adapalene, Ch55, and the like. In some embodiments, compositions disclosed herein for culturing DE cells to generate AFE cells and/or for culturing AFE cells to generate VPE cells according to methods of the present disclosure can include a media having a high protein concentration of at least 0.5% (w/v) protein and at least one retinoic acid signal activating agent (and optionally, heparin, at least one BMP activator, and/or at least one FGFR3 activator) where the retinoic acid signal activating agent can be present in compositions disclosed herein at a concentration ranging from about 0.1 nM/mL to about 3,000 nM/mL; or any concentration in between in order to obtain the desired signaling response. In some embodiments, a retinoic acid signaling activator can be present in compositions disclosed herein at a concentration of about 0.1 nM, to about 3.0 μM.

In certain embodiments, compositions disclosed previously for culturing DE cells to generate AFE and/or PE cells and/or for culturing AFE and/or PE cells to generate VPE cells according to methods of the present disclosure can include a media having a high concentration of protein and at least one of at least one BMP activating agent, at least one FGFR3 activating agent, at least one FGFR2 activating agent, at least one NOTCH activating agent, or a combination thereof. In certain embodiments, compositions disclosed herein for culturing DE cells to generate AFE and/or PE cells and/or for culturing AFE and/or PE cells to generate VPE cells according to methods herein can include media having a high concentration of protein and at least one of at least one BMP activating agent, at least one FGFR3 activating agent, at least one FGFR2 activating agent, at least one NOTCH activating agent, at least one agent that activates retinoic acid receptor signaling, or a combination thereof.

In certain embodiments, compositions for culturing DE cells to generate AFE cells and/or for culturing AFE cells to generate VPE cells do not include a transforming growth factor-β (TGF-β) modulating agent. In certain embodiments, compositions disclosed herein for culturing DE cells to generate AFE cells and/or for culturing AFE cells to generate VPE cells do not include a TGF-β inhibitor.

In certain embodiments, compositions for culturing DE cells to generate AFE cells and/or for culturing AFE cells to generate VPE cells can replace a cell culture media after the previous culture medium is removed and optionally, washed to remove remaining agents. In accordance with these embodiments, compositions disclosed herein for culturing DE cells to generate AFE cells and/or for culturing AFE cells to generate VPE cells can be provided to the cell culture after the previous culture medium is removed with or without a wash step before application.

In certain embodiments, compositions for culturing DE cells to generate AFE cells and/or for culturing AFE cells to generate VPE cells can be provided to the cell culture and incubated for a period of time suitable for DE cells to differentiate into AFE cells. In some embodiments, compositions disclosed herein for culturing DE cells to generate AFE cells and/or for culturing AFE cells to generate VPE cells can be provided to DE cells to differentiate into AFE cells. In some embodiments, compositions disclosed herein for culturing DE cells to generate AFE cells and/or for culturing AFE cells to generate VPE cells.

Stage 5—Culturing VPE or Third Pharyngeal Pouch Endoderm (TPPE) Cells to Generate Thymic cells to Generate sTOs

In certain embodiments, compositions can be used to provide conditions for culturing VPE and/or TPPE cells to generate thymic cells (TEC and or TEP cells) according to methods previously disclosed and that are available in the art. In accordance with these embodiments, compositions disclosed herein can include a base medium having a high or enriched concentration of protein (e.g., a “protein enriched medium”). In some embodiments, high protein concentrations can include at least about 0.5% (w/v) to about 30% (w/v).

In some embodiments, compositions for culturing TPPE cells to generate thymic cells according to methods of the present disclosure can include a protein rich media and at least one BMP modulating agent. In some embodiments, compositions for culturing TPPE cells to generate thymic cells according to methods of the present disclosure can include a protein rich media and at least one BMP inhibitor. In some embodiments, compositions disclosed herein for culturing VPE and/or TPPE cells to generate thymic cells according to methods of the present disclosure can include a protein rich media and at least one BMP inhibitor at a concentration of at least about 5 ng/ml to about 1,000 ng/mL.

In some embodiments, compositions for culturing VPE and/or TPPE cells to generate thymic cells according to methods previously disclosed can include a protein enriched media with at least one Activin A modulating agent. In some embodiments, compositions disclosed herein for culturing VPE and/or TPPE cells to generate thymic cells according to methods of the present disclosure can include a protein-rich media with at least one Activin A activator. In some embodiments, compositions disclosed herein for culturing VPE and/or TPPE cells to generate thymic cells according to methods of the present disclosure can include a protein-rich media having at least 0.5% (w/v) protein up to about 30% (w/v) protein and at least one Activin A activator at a concentration of at least about 5 ng/mL to at least about 1000 ng/mL, for example. In certain embodiments, compositions disclosed herein for culturing VPE and/or TPPE cells to generate thymic cells according to methods of the present disclosure can include a protein-rich media having at least 0.5% (w/v) protein up to about 30% (w/v) protein and at least one retinoic acid signaling modulating agent (and optionally at least one Activin A activator and/or BMP inhibitor).

In certain embodiments, compositions for culturing VPE and/or TPPE cells to generate thymic cells according to methods of the present disclosure can include a protein-rich media having at least 0.5% (w/v) protein up to about 30% (w/v) protein and further includes at least one fibroblast growth factor receptor (FGFR) modulating agent. In some embodiments, compositions disclosed herein for culturing VPE and/or TPPE cells to generate thymic cells according to methods of the present disclosure can include a protein-rich media having at least 0.5% (w/v) protein up to about 30% (w/v) protein and further includes at least one FGFR activator (and optionally at least one Activin A activator, at least one BMP inhibitor, at least one retinoic acid receptor activator, and/or heparin).

In certain embodiments, compositions for culturing VPE and/or TPPE cells to generate thymic cells according to methods of the present disclosure can include a base medium with at least one hedgehog signaling modulating agent, including sonic hedgehog (Shh) As used herein, “a Shh modulating agent” can refer to any chemical (e.g., small molecule or other chemical agent), compound, polypeptide, protein or fragment thereof, polynucleotide (DNA or RNA), or other agent that modulates hedgehog and/or Shh and/or a Shh signaling pathway. In some embodiments, compositions disclosed herein for culturing VPE and/or TPPE cells to generate thymic cells according to methods of the present disclosure can include a protein-rich media having at least 1% (w/v) protein up to about 30% (w/v) protein and further includes at least one Shh inhibitor (and optionally at least one: at least one Activin A activator, at least one BMP inhibitor and at least one retinoic acid receptor activator, at least one FGFR2 activator and heparin). Non-limiting Shh inhibitors suitable for use herein can include SANT1, SANT2, U1866A, Dynapyrazole a, Dynarrestin, Cyclopamine, HIP1, GANT58, AY9944 dihydrochloride, RU-SKI 43 hydrochloride, and the like.

In certain embodiments, compositions for culturing VPE and/or TPPE cells to generate thymic cells according to methods of the present disclosure can include a base medium having an enriched protein concentration and at least one of, at least one NOTCH pathway signaling activator, at least one CD40 pathway signaling activator, and/or at least one RANK signaling pathway activator. In accordance with these embodiments, compositions disclosed herein having at least one NOTCH pathway activator can include, but are not limited to, Yhhu 3792, DLL1, DLL3, DLL4, Jagged 1, and Jagged 2. In certain embodiments, compositions disclosed herein can include or further include at least one CD40 pathway activator. In accordance with these embodiments, the at least one CD40 pathway activator can include, but are not limited to, CD40 ligand, multimeric CD40 ligand, CD40 ligand and a crosslinker, and an anti-CD40 antibody with an antibody crosslinker. In some embodiments, compositions disclosed herein can include or further include at least one RANK pathway activator. In accordance with these embodiments, the at least one RANK pathway activator can include. but are not limited to, soluble RANK ligand, RANK ligand, multimeric RANK ligand, RANK ligand and a crosslinker, and an anti-RANK antibody with an antibody crosslinker. In other embodiments, compositions disclosed herein can further include at least one Ghrelin receptor pathway activator. In accordance with the embodiments, the at least one Ghrelin receptor pathway activator can include, but are not limited to, Ghrelin, MK 0677, Tabimorelin hemifumerate, and L-692,585. In certain embodiments, compositions disclosed herein can include at least one NOTCH pathway activator and at least one of, at least one CD40 pathway activator and at least one RANK pathway activator. In accordance with these embodiments, compositions disclosed herein can include a protein enriched culture medium (e.g., MEM alpha media).

In certain embodiments, compositions for culturing VPE and/or TPPE cells to generate thymic cells according to methods previously disclosed can include a protein-enriched media having at least 0.5% (w/v) protein up to about 30% (w/v) protein and further includes at least one Epidermal Growth Factor (EGF) (and optionally at least one: at least one Activin A activator, at least one BMP inhibitor, at least one retinoic acid receptor activator, at least one FGFR2 activator, at least one Shh inhibitor, and/or heparin). In some embodiments, compositions disclosed herein for culturing VPE and/or TPPE cells to generate thymic cells can include a protein-rich media having at least 0.5% (w/v) protein up to about 30% (w/v) protein and further includes at least one Epidermal Growth Factor (EGF) (and optionally at least one: at least one Activin A activator, at least one BMP inhibitor and at least one retinoic acid receptor activator, at least one FGFR2 activator, at least one Shh inhibitor and heparin) where the EGF is present at concentrations ranging from about 0.1 ng/mL to about 30 ng/mL.

In certain embodiments, compositions for culturing VPE and/or TPPE cells to generate thymic cells according to methods of the present disclosure can include a protein enriched media and optionally, include at least one Wnt modulating agent. In some embodiments, compositions disclosed herein for culturing VPE and/or TPPE cells to generate thymic cells according to methods of the present disclosure can be in a protein rich media optionally including at least one Wnt activator. In some embodiments, compositions disclosed herein for culturing VPE and/or TPPE cells to generate thymic cells can be a base medium optionally including at least one Wnt activator.

In certain embodiments, compositions for culturing VPE and/or TPPE cells to generate thymic cells as previously disclosed can include a protein enriched media optionally including at least one CD40 activating agent. In some embodiments, compositions disclosed herein for culturing VPE and/or TPPE cells to generate thymic cells can include a protein rich media optionally including at least one CD40 activator. In certain embodiments, compositions for culturing VPE and/or TPPE cells to generate thymic cells can include a protein enriched media optionally including at least one Ghrelin receptor signaling activating agent. In certain embodiments, compositions for culturing VPE and/or TPPE cells to generate thymic cells can include a base medium enriched in protein and at least one Activin A modulating agent, at least one BMP modulating agent, at least one FGFR modulating agent, at least one a retinoic acid signaling modulating agent, at least one Shh modulating agent, optionally a Wnt activator and/or NOTCH activator or a combination thereof.

In certain embodiments, one or more additional supplements can also be added to any of the compositions described herein to supply the cells with trace elements for improved growth and expansion. In accordance with these embodiments, additional supplements can be used compositions used in one or more stages of differentiation for producing thymic cells from PSCs. Such supplements can include, but are not limited to, insulin, transferrin, sodium selenium, and combinations thereof. β-mercaptoethanol and other hormones are further contemplated herein. These components can be included in any known acceptable form. In some embodiments, these agents can be in a salt solution including, but not limited to, Hanks' Balanced Salt Solution® (HBSS), Earle's Salt Solution®, antioxidant supplements, MCDB-201® supplements, phosphate buffered saline (PBS), N-2-hydroxyethylpiperazine-N′-ethanesulfonic acid (HEPES), nicotinamide, ascorbic acid, and/or ascorbic acid-2-phosphate, as well as additional amino acids. In some examples, amino acids for use herein can include, but are not limited to, L-alanine, L-arginine, L-aspartic acid, L-asparagine, L-cysteine, L-cystine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

In other aspects, as needed, antibiotics can be used in cell cultures to mitigate or eliminate bacterial, mycoplasma, and fungal contamination. Typically, antibiotics or anti-mycotic compounds used are mixtures of penicillin/streptomycin, but can also include, but are not limited to, amphotericin (Fungizone®), ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be used in cell cultures and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine.

Lipids and lipid carriers can also be used to supplement cell culture media, depending on the type of cell and the fate of the differentiated cell. Such lipids and carriers can include, but are not limited to cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

In some embodiments, compositions herein can include a base medium supplemented with an Insulin-Transferrin-Selenium (ITS) supplement. In accordance with these embodiments, ITS can be provided to the medium in concentrations ranging from about 1:10 (v/v) to about 1:10,000 (v/v). In some embodiments, compositions herein can include a base medium supplemented with cortisol (e.g., hydrocortisone). In accordance with these embodiments, cortisol can be provided to the medium in concentrations ranging from about 0.05 μg/mL to about 5 μg/mL.

In certain embodiments, cell populations cultured for use in the humanized mouse models (e.g., to generated sTOs from TECs) or cultured from the humanized mouse model after creation of the patient-specific humanized mouse can be monitored to assess changes in the cells imparted by culturing (e.g., during a stage of the culture method disclosed herein) to characterize the cell population produced. In some embodiments, the production of third pharyngeal pouch endoderm (TPPE) cells, TEP cells, and/or TECs or the like, including various sub-populations of TECs, can be assessed by determining the expression of markers characteristic of these cell populations. In some embodiments, thymic cells (e.g., TEPs/TECs) cell-identifying markers can include SOX 17⁻, SOX2⁺ and/or FOXA2⁺; EPCAM+CD104⁺; EPCAM⁺ CD205⁺; or any combination thereof. In certain embodiments, thymic cells (e.g., TEPs/TECs) cell-identifying markers can include EPCAM⁺ CD104⁺ where more than about 5%, more than about 8%, more than about 10%, more than about 11%, more than about 12%, more than about 13%, more than about 14%, or more than about 15% of the cells can be identified as EPCAM⁺ CD104⁺. In some embodiments, thymic cells (e.g., TEPs/TECs) cell-identifying markers can include EPCAM⁺ CD205⁺ where more than about 55%, more than about 56%, more than about 57%, more than about 58%, more than about 59%, more than about 60%, more than 61%, more than 62%, more than 63%, more than 64%, or more than 65% of the cells can be identified as EPCAM⁺ CD104⁺. In accordance with these embodiments, expression of cell markers can be determined by detecting the presence or absence of the marker, and/or by analyzing expression of certain markers by measuring the level at which the marker is present in the cells of the cell culture or cell population. Methods known in the art can also be used to quantitate marker gene expression. Non-limiting examples of methods suitable for use herein can include PCR, RT-PCR, qRT-PCR, immunoblotting, immunofluorescence, enzyme-linked immunosorbent assay (ELISA), flow cytometry, and the like.

In certain embodiments, methods disclosed herein can include transplanting sTOs produced according to the methods disclosed herein into a mouse alone or in combination with one or more cells that are not thymic cells. In accordance with these embodiments, thymic cells generated according to the methods disclosed herein can be transplanted into a subject in combination with lymphatic endothelium cells, vascular endothelium cells, immune cells, mesenchymal cells, pericytes, red blood cells, or any combination thereof. In some embodiments, methods disclosed herein can include transplanting thymic cells generated according to the methods disclosed herein into a humanized mouse in combination with one or more patient-specific cells for humanizing the mouse.

For the purposes described herein, autologous thymic cells prepared according to methods of the present disclosure can be introduced to a humanized mouse. In accordance with these embodiments, the thymic cells can be either in undifferentiated, partially differentiated, or fully differentiated forms, genetically altered or unaltered, introduced by direct injection to a tissue site, by infusion through a portal vein, in a bolus delivered to an organ, administered systemically, on or around the surface of an acceptable matrix, encapsulated or in combination with a pharmaceutically acceptable carrier.

In some embodiments, thymic cells prepared according to the methods disclosed herein can be introduced to a humanized mouse over the course of a day, for a few hours, daily, every other day, 2 times per week, weekly, every other week, monthly, or other appropriate treatment regimen.

In some embodiments, one or more pharmaceutical agents can be assessed for efficacy to inhibit tumor expansion and/or to reduce or eliminate a tumor or reduce metastasis of a tumor in the patient specific humanized mouse model. In accordance with these embodiments, the one or more pharmaceutical agents can include, but are not limited to immune checkpoint modifying agents, including, but not limited to, targeted antibodies, snRNA or the like (e.g., antibodies to immune checkpoint inhibitors such as PD-1, etc.). In other embodiments, the one or more pharmaceutical agents can include, but are not limited to, one or more immunosuppressive agents. In some embodiments, the one or more pharmaceutical agents can include, but are not limited to, one or more of an antibody to one or more of PD-1, PD-L1, CTLA4. In accordance with these embodiments, the one or more antibodies can include, but is not limited to, Pembrolizumab, Nivolumab, Cemiplima, Dostarlimab, Retifanlimab, Atezolizumab, Avelumab, Durvalumab, Ipilimumab, Tremelimumab, and the like. In other embodiments, the one or more pharmaceutical can include, but is not limited to, one or more cell therapies. In accordance with these embodiments, the one or more cell therapies can include, but are not limited to, Sipuleucel-T, Lifileucel, or similar agent.

The therapeutic agents described herein can be administered as pharmaceutical compositions and can be provided as liquid preparations, e.g., isotonic aqueous solutions, suspensions, emulsions, or viscous compositions, which can be buffered to a selected pH. The choice of suitable carriers and other additives can depend on the route of administration and the nature of the particular dosage form, e.g., liquid dosage form (e.g., whether the composition is to be formulated into a solution, a suspension, gel or another liquid form, such as a time release form or liquid-filled form). Solutions, suspensions and gels normally contain a major amount of water (e.g., purified, sterilized water) in addition to the cells. Minor amounts of other ingredients such as pH adjusters (e.g., a base such as NaOH or an acid such as HCl), emulsifiers or dispersing agents, buffering agents, preservatives, wetting agents and jelling agents (e.g., methylcellulose), can also be present. In some embodiments, pharmaceutical compositions contemplated herein can be isotonic, i.e., can have the same osmotic pressure as blood and lacrimal fluid. In other embodiments, agents can be provided to reduce cell lysing or other adverse effect on the cells for delivery to a subject.

A pharmaceutically acceptable preservative or cell stabilizer can be employed to increase the life of the compositions. If preservatives are used, it is well within the purview of the skilled artisan to select compositions that will not affect the viability or efficacy of the cells as described herein.

### EXAMPLES

The following examples are included to illustrate certain embodiments. It should be appreciated by those of skill in the art that the techniques disclosed in the examples which follow represent techniques discovered to function well in the practice of the claimed methods, compositions, and apparatus. However, those of skill in the art should, considering the present disclosure, appreciate that changes can be made in some embodiments which are disclosed and still obtain a like or similar result without departing from the spirit and scope of the invention.

### Example 1

In Vitro Development and Differentiation of sTOs Gives Rise to T Cells

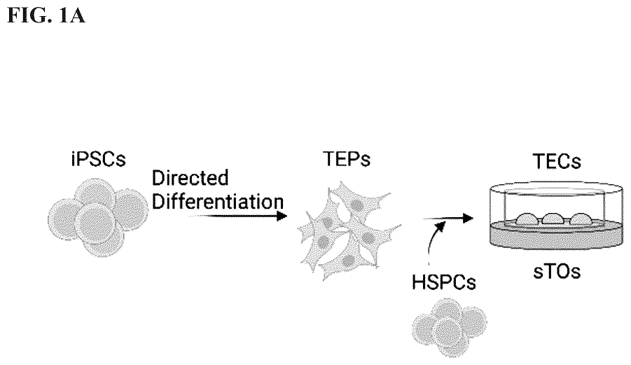

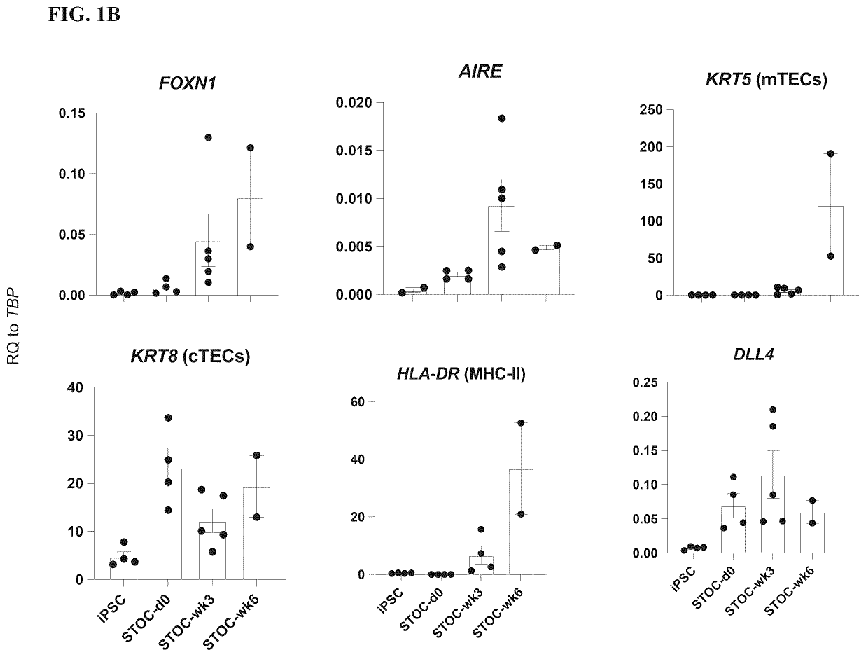

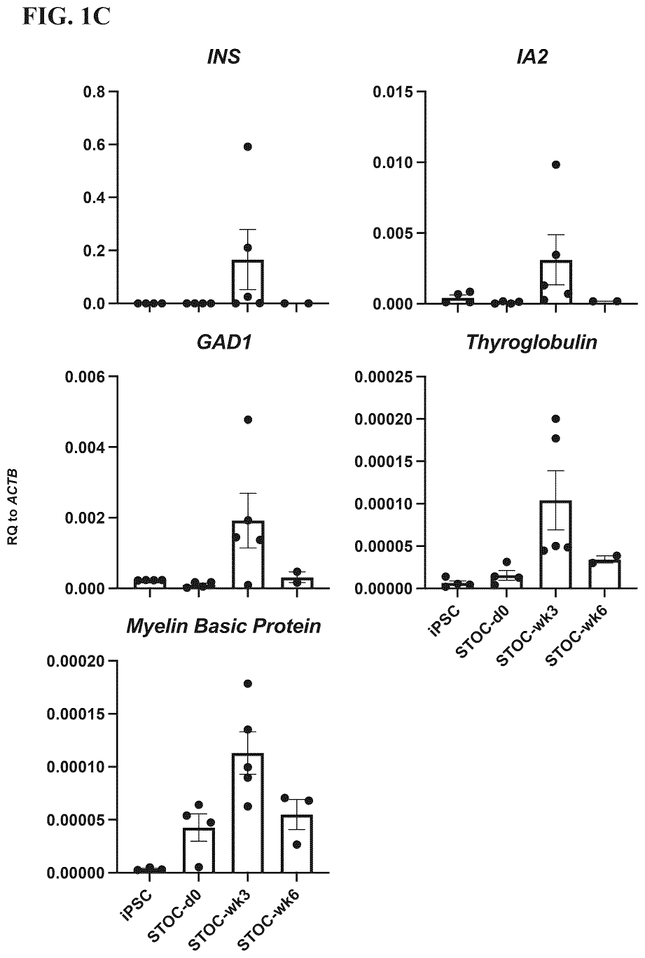

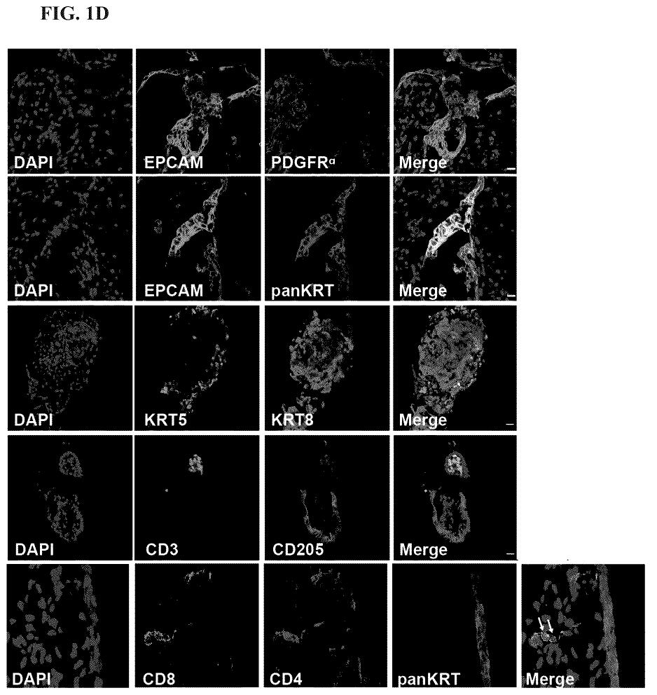

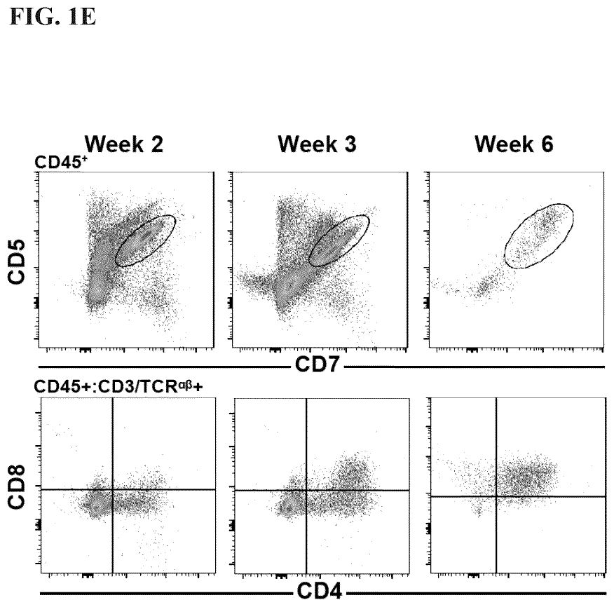

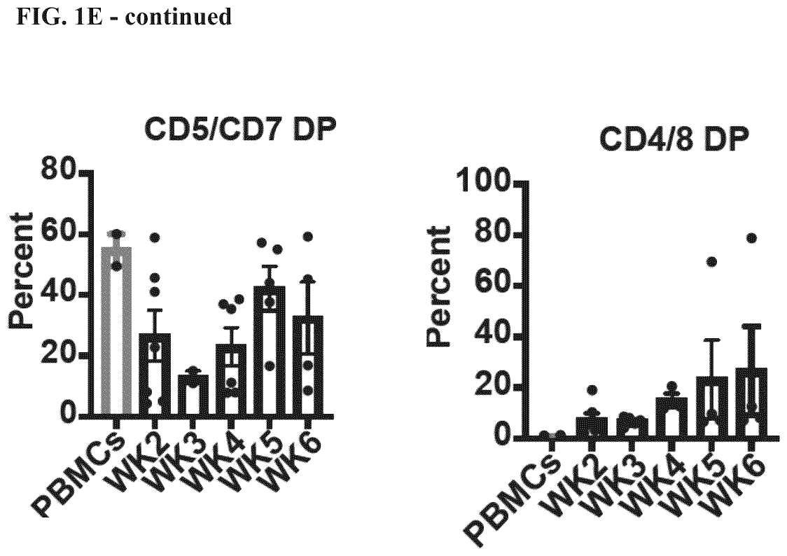

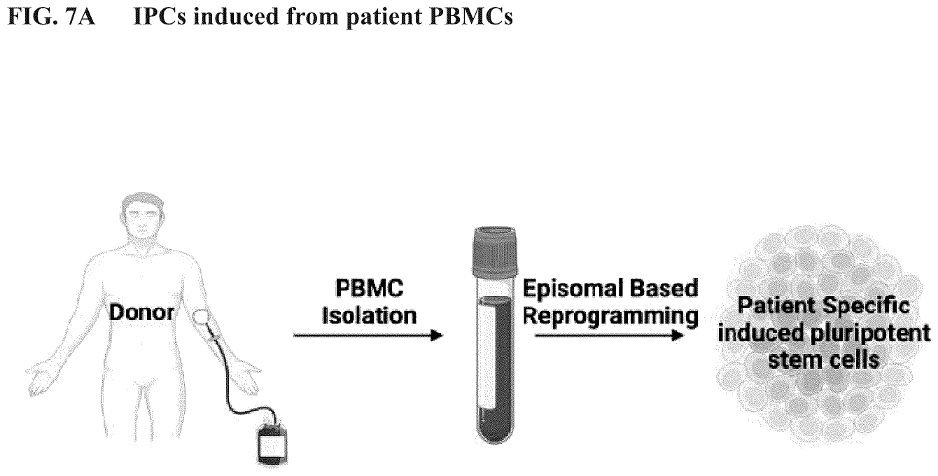

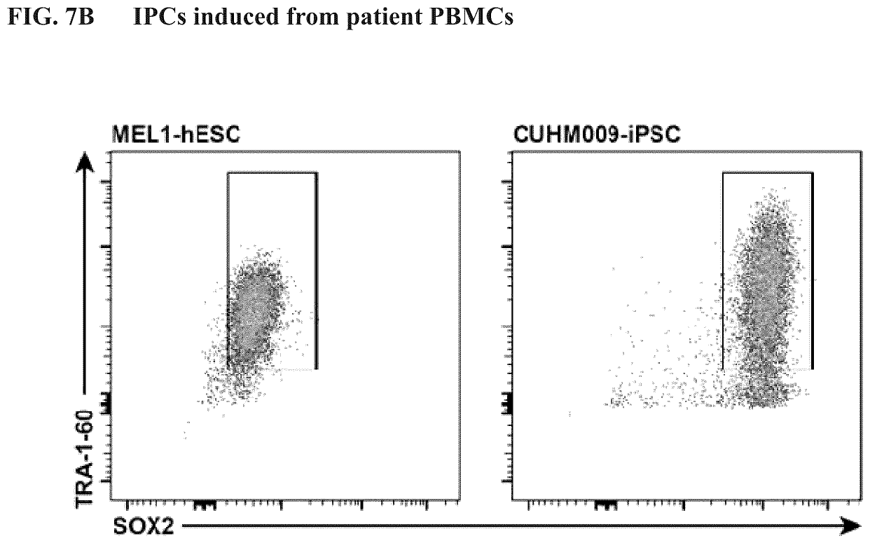

In one exemplary method, PBMCs of a melanoma patient (CUHM009) were reprogrammed into iPSCs by episomal reprogramming, with TRA-1-60 and SOX2 expression demonstrated to be comparable to the previously characterized Mell-hESC cell line to verify successful reprogramming (FIG. 7A-7B). In further exemplary methods, these patient-specific iPSCs were used to generate TEPs via direct differentiation, as previously described (Ramos S A et al., J Allergy Clin Immunol, 2021 and Ramos S A et al., Stem Cell Reports, 2023, both incorporated herein by reference in their entireties for all purposes). Stem cell-derived thymic organoids (sTOs) were then established by aggregating patient iPSC-derived TEPs with allogenic, human cord blood-derived CD34⁺ hematopoietic stem and progenitor cells (HSPCs) at the air-liquid interface (FIG. 1A). Subsequent qPCR analysis of sTOs showed the upregulation of key thymic markers Forkhead Box N1 (FOXN1), autoimmune regulator (AIRE), keratin 5 (KRT5), keratin 8 (KRT8), major histocompatibility complex, class II, DR alpha (HLA-DRA), and delta like canonical Notch ligand 4 (DLL4) after 3 weeks and/or six weeks, indicating the maturation of iPSC-derived TEPs to TECs in vitro (FIG. 1B). The trend toward increased expression of the AIRE transcripts in sTOs after 3 weeks in culture was not previously observed in vivo using the xenogeneic nude model, previously disclosed. Further qPCR analysis identified expression of the tissue-restricted antigens (TRAs) INS, IA2, GAD1, Myelin Basic Protein and Thyroglobulin in the sTOs starting at 3 weeks, corroborating the AIRE expression (FIG. 1C).

Immunofluorescent (IF) analysis of sTO sections for epithelial and mesenchymal markers, EPCAM and PDGFRα respectively, revealed presumptive thymic epithelial structures surrounded by mesenchymal cells (FIG. 1D, top row). Co-staining of EPCAM″ cells with a pan-Keratin antibody showed co-expression in almost all epithelial cells (FIG. 1D, second row). To identify medullary and cortical (m-/cTECs), sections were stained for KRT5 and 8, respectively. Some KRT5/8 double positive cells, indicative of developing TECs (FIG. 1D, third row), were present. Additionally, CD3⁺ lymphocytes and CD205⁺ TECs were present in sTOs, indicating the development of distinct epithelial and lymphocyte compartments in them (FIG. 1D, fourth row). Further IF analysis of sTO sections for T-cell markers CD4 and 8 showed double positive, (DP) developing T-cells located near pan-KRT positive thymic structures (FIG. 1D, bottom row).

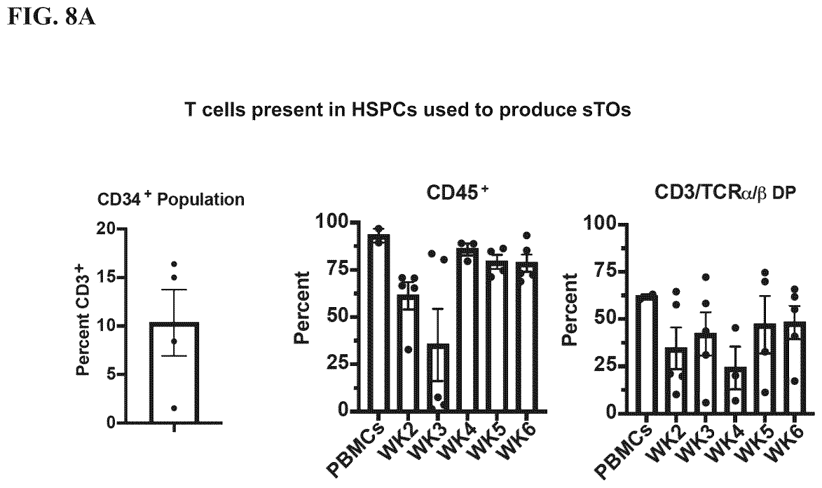

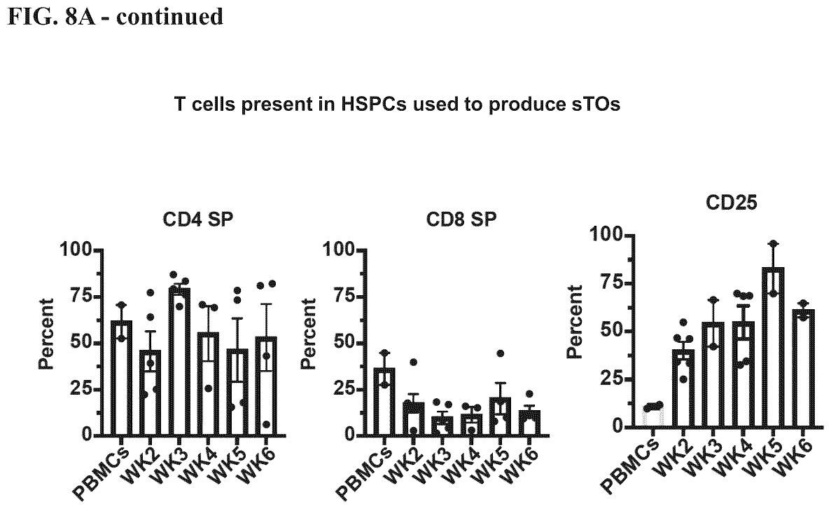

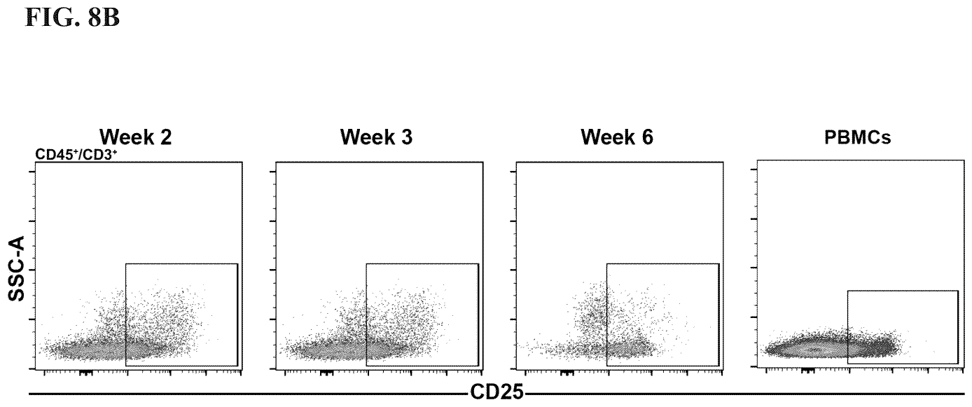

The emergence of pro T-cells, marked by CD5 and CD7, was quantified by flow cytometry and found low levels of pro T-cells at week 2 followed by a substantial increase at week 3 and throughout the culture period assayed (FIG. 1E, top row). Quantitative flow analysis of CD4 and 8 revealed increasing amounts of developing DP T-cells starting at week 2, which were not present in isolated CD34⁺ HSPCs at the start of sTO culture (FIG. 1E, middle row). Large populations of single positive (SP) CD4 and 8 T-cells were also observed at weeks 2 and 3 (data not shown). Further analysis of isolated CD34⁺ HSPCs showed a small population of contaminating CD3⁺ T-cells that could potentially become activated and expand considerably in culture after prolonged culture (FIG. 8A-8B), thus precluding the identification of de novo generation of single positive T-cells in this allogenic sTO system with high confidence.

### Example 2

HLA-Matched HM Implanted with sTOs

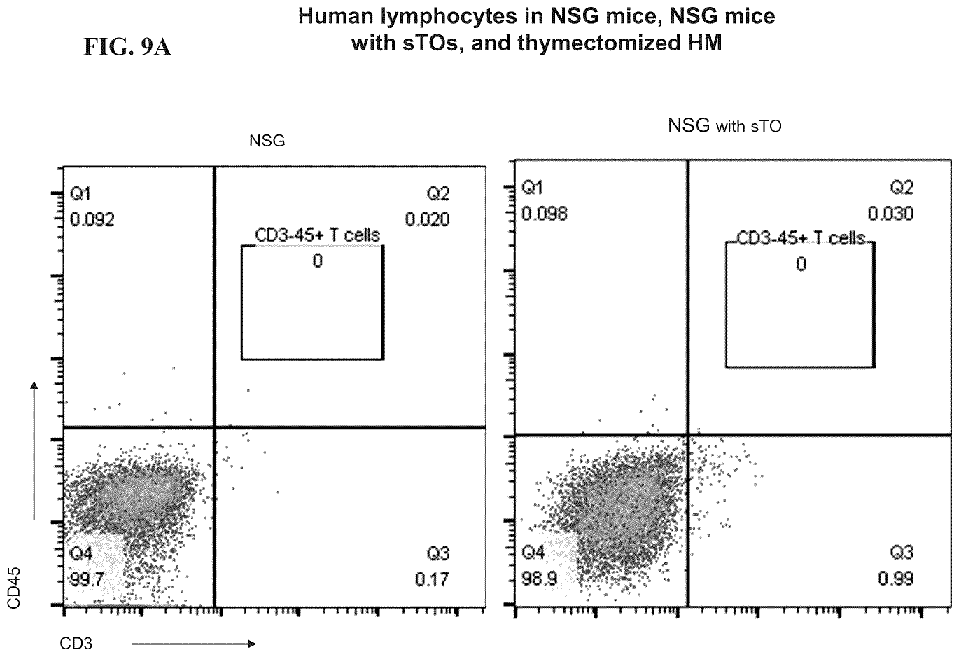

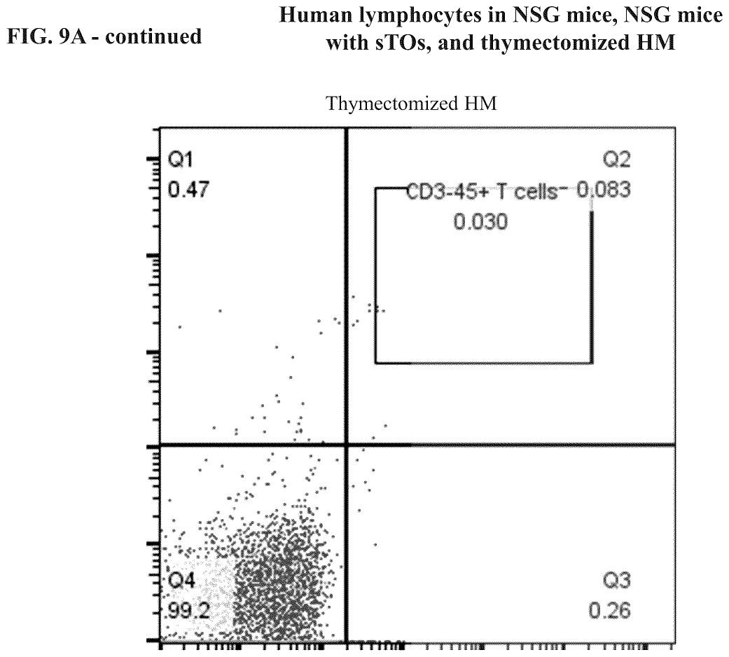

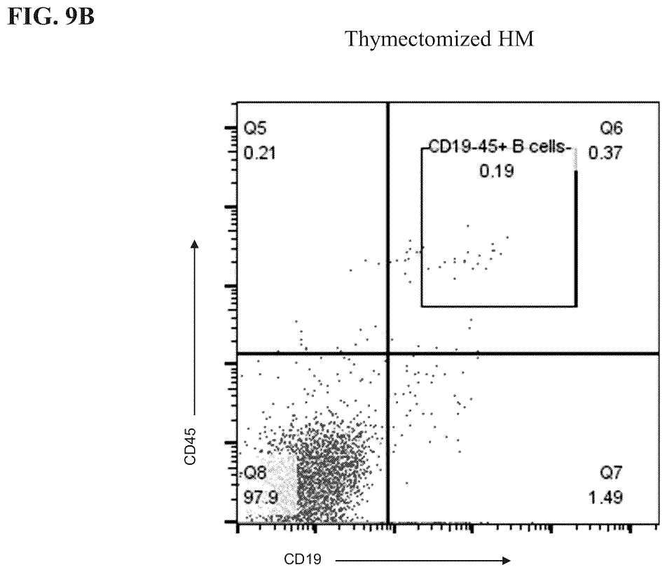

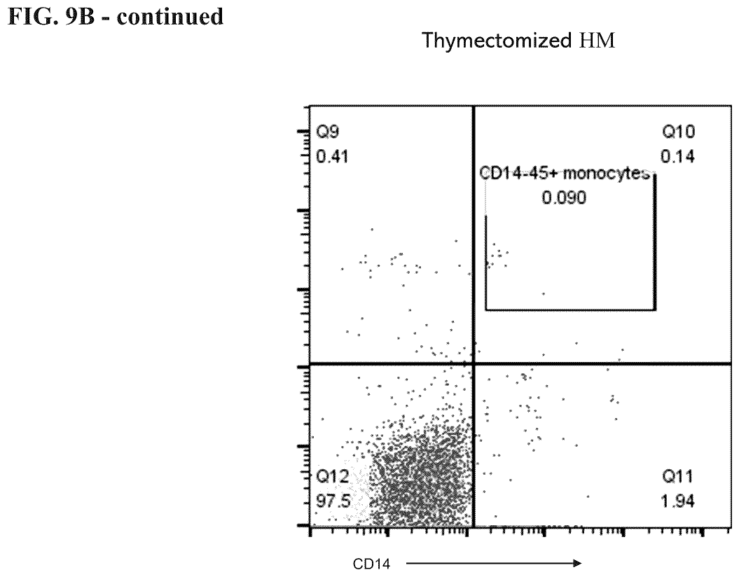

In another exemplary method, humanized mice (HM) were generated to test the sTO capacity to produce physiologically active and immune-relevant T-cells in vivo by comparing PDX tumor growth on 1) HLA-A matched HM in which T-cells were educated in the murine thymus (mHM), 2) thymectomized mHM with patient-autologous sTOs implanted under the kidney capsule (mHM_{TA}), and 3) non-humanized NOD scid gamma (NSG) controls. As additional controls, sTOs were implanted in non-humanized NSGs, and thymectomized mHM but did not subsequently implant sTOs. No T cells were present in non-humanized NSG mice with sTO implants, and only very few putative T cells were observed in thymectomized HM with no sTO implants (FIG. 9A), although these animals did harbor measurable B cell and monocyte populations (FIG. 9B). In further exemplary methods, umbilical cord blood donors that matched one HLA-A allele of the PDX were selected. Genomic DNA samples from CUHM009 patient blood samples were submitted for high resolution HLA typing (Table 1, below).

TABLE 1
High Resolution HLA Typing Results

| Locus | UCB120 | UCB122 | CUHM009 |
|---|---|---|---|
| HLA-A | *02:01/*25:01 | *02:01/*68:01 | *02:01/*24:02 |
| HLA-B | *15:01/*51:01 | *39:06/*40:02 | *44:02/*44:02 |
| HLA-DQA-1 | *01:03/*01:03 | *03:01/*05:03 | *01:02/*03:03 |
| HLA-DQB-1 | *06:03/*06:03 | *03:02/*03:01 | *03:01/*06:04 |
| HLA-DRB-1 | *13:01/*13:01 | *04:07/*14:06 | *04:01/*13:02 |

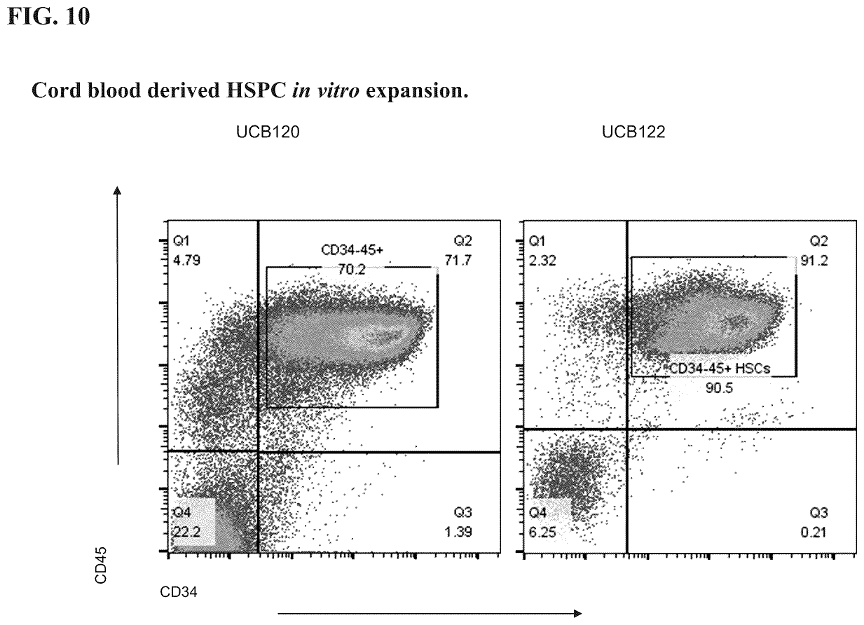

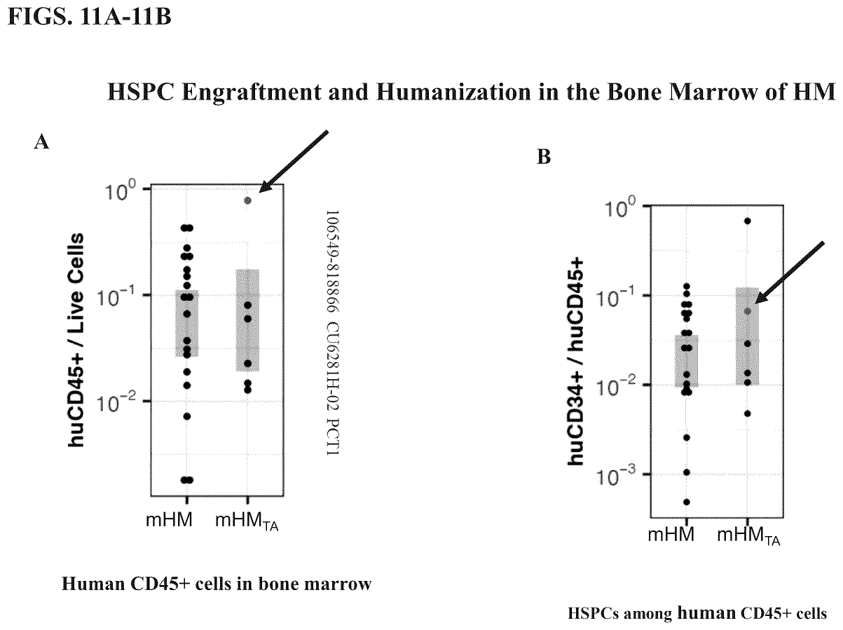

Since CUHM009 had a single HLA-A*02:01 allele, one HLA-A*02:01+/− donor was identified for each cohort, UCB120 and UCB122, and Hematopoietic Stem and Progenitor Cells (HSPCs) were isolated then expanded in vitro. After 9 and 8 days of expansion, respectively, the UCB 120 culture had 70.2% CD34⁺/CD45⁺ HSPCs while the UCB 122 culture had 90.5% CD34⁺ HSPCs (FIG. 10). In further exemplary methods, HSPC engraftment and subsequent humanization were measured by enumerating the fraction of human CD45⁺ cells in the bone marrow at the end of the study. No statistically significant differences between any experimental groups were found (Wilcoxon Test, p-values from 0.45-0.84). Similarly, there was no significant difference in the fraction of human CD34⁺ HSPCs relative to total human CD45⁺ cells (Wilcoxon Test, p-values from 0.17-0.88) (FIG. 11A-11B).

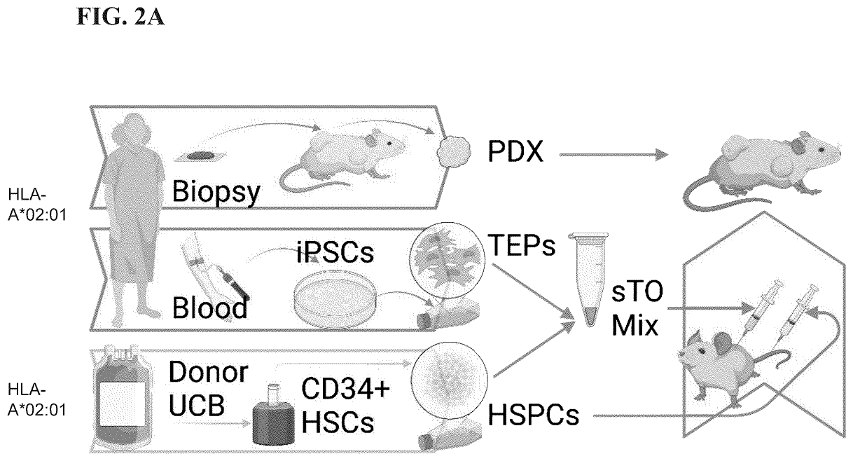

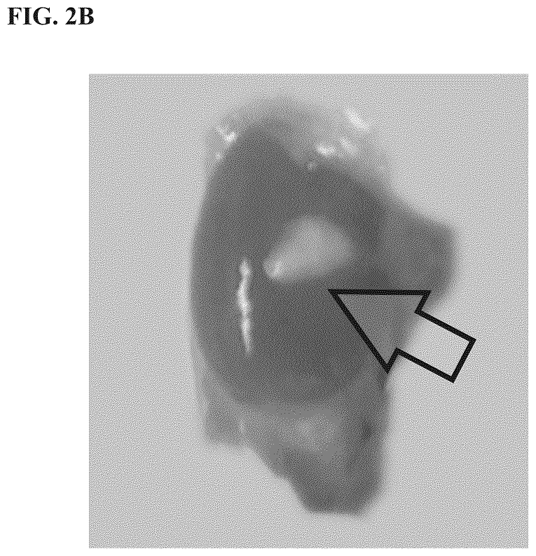

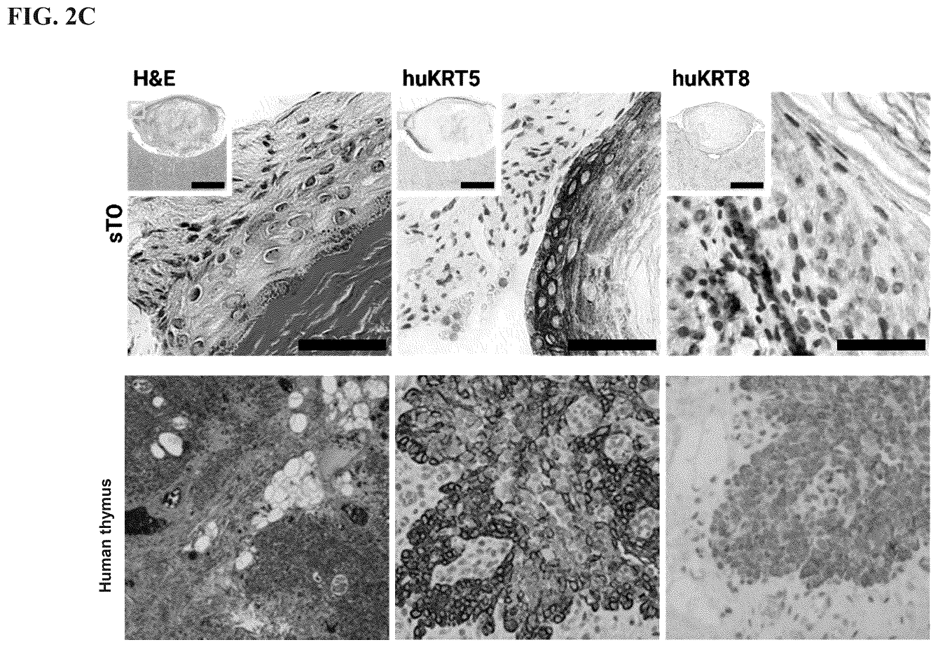

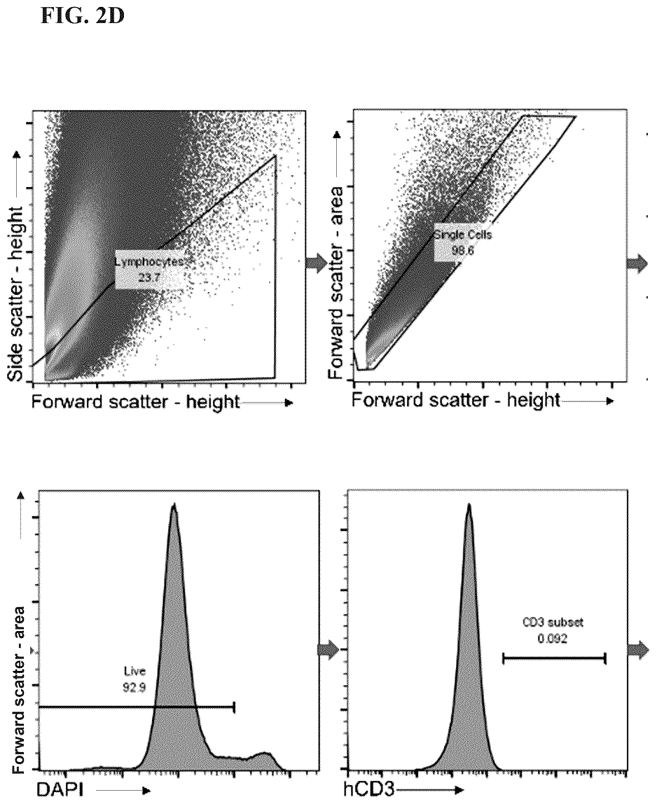

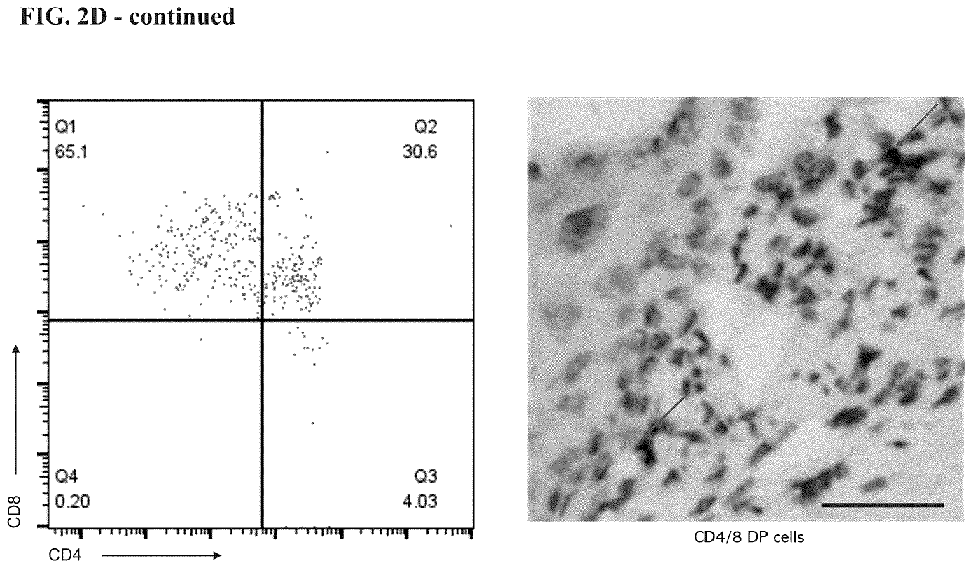

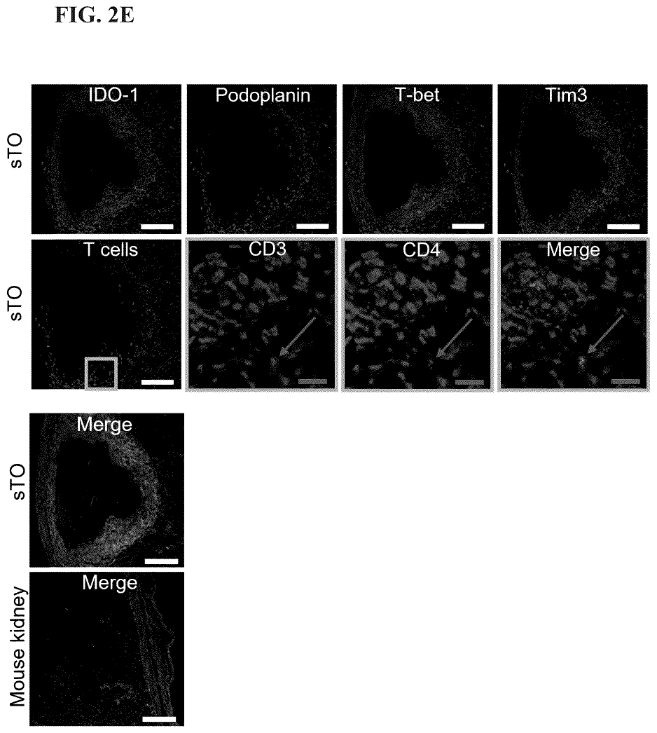

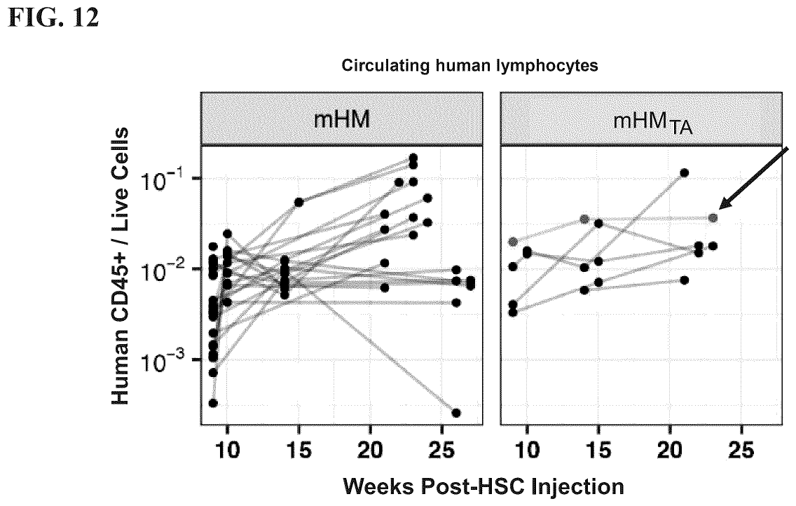

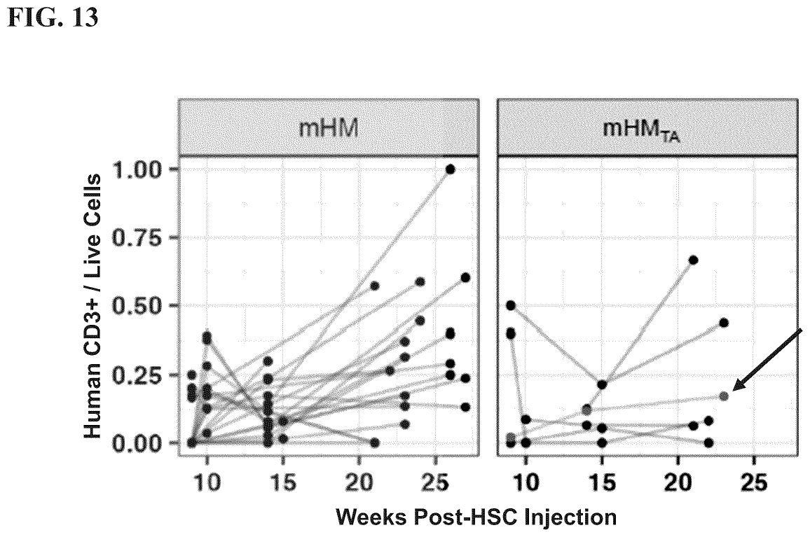

In further exemplary methods, to examine sTOs in vivo, CUHM009 sTOs were mixed with HLA-A-matched HSPCs and implanted under the kidney capsules of mHM_{TA} previously humanized with the same HSPCs. Circulating human lymphocytes were then monitored following humanization and during tumor growth (FIG. 2A). For all mice, human hematopoietic lineages begin to appear in the peripheral blood nine weeks post-HSPC engraftment and gradually increased from this point to the end of the study, that occurred 20-25 weeks post-engraftment (linear mixed effects model, p=0.02) (FIG. 12). CD3⁺/45⁺ T-cells in the mHM and mHM_{TA} group were first observed approximately nine weeks post-engraftment and increased throughout the study (FIG. 13).

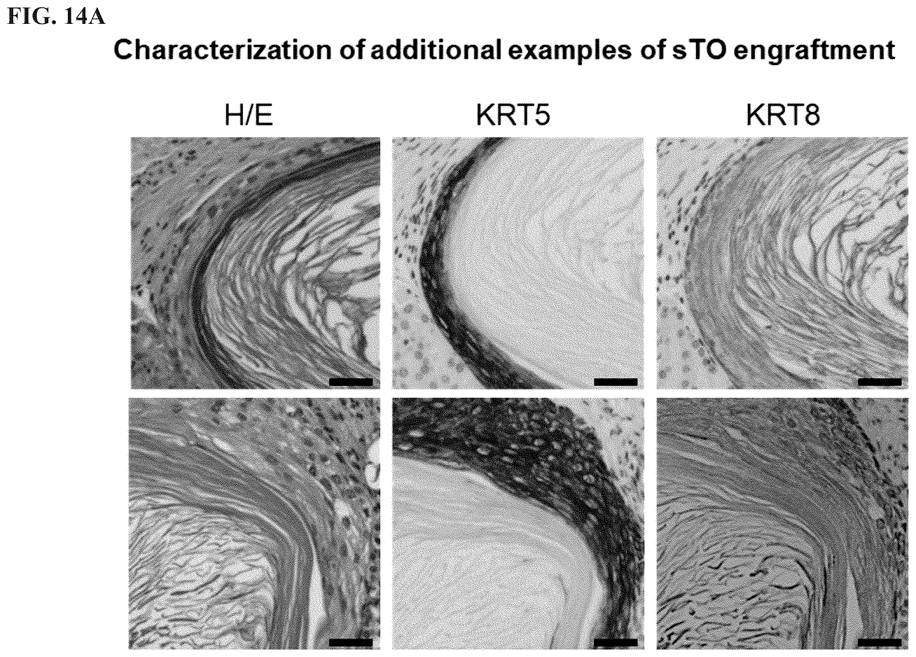

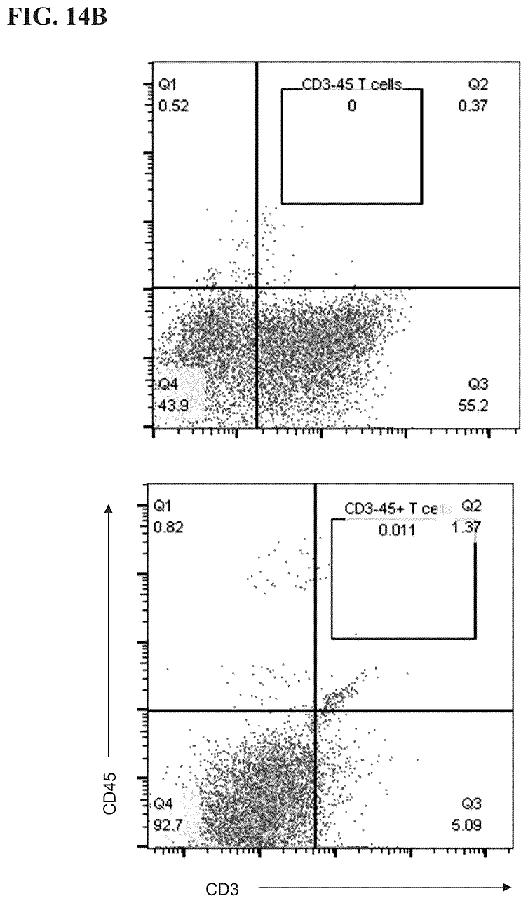

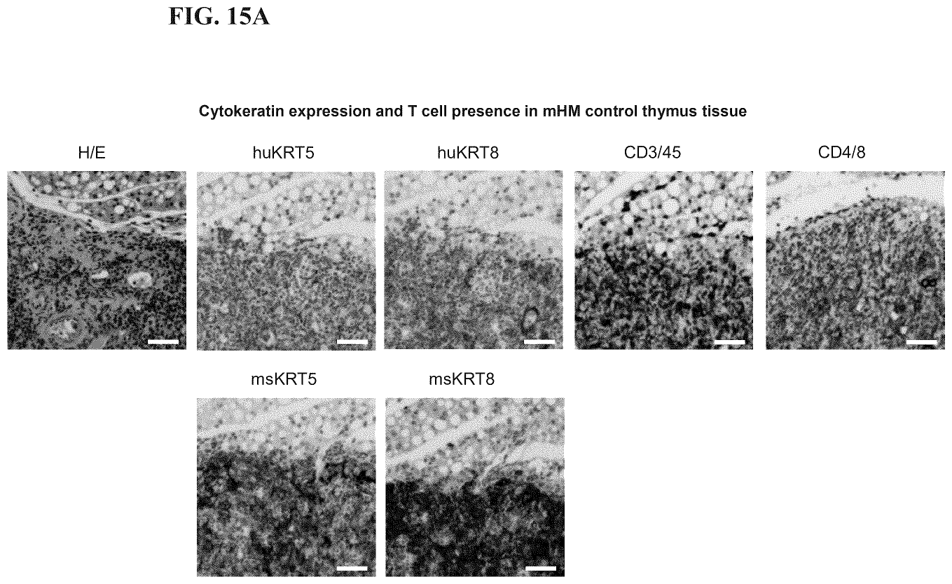

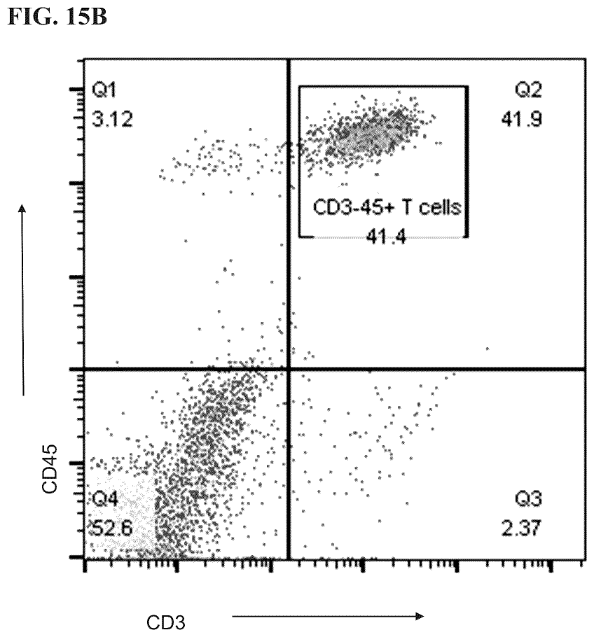

At harvest, engrafted sTOs had grown into thymic-like structures within the kidney capsule (FIG. 2B). At necropsy, six mice (three from UCB120 and three from UCB122) were identified with confirmed sTOs in the renal capsule, as assessed by IHC with human KRT5 and KRT8 staining (FIG. 2C and FIGS. 14A-14B). In contrast, murine thymus tissue removed from mHM expressed only murine cytokeratins and lacked expression of any of the human thymic markers, although it did contain human T cells and maturing DP T cells (FIG. 15). No mature, developed sTO were recovered from NSG controls.

### Example 3

mHMTA Model Produces Physiologically Relevant T-Cell Populations

In another exemplary method, CD4⁺/CD8⁺ DP T-cells were measured in the sTOs by flow cytometry and IHC. MIBI was used for sTO histological characterization and comparison to a nongrafted mouse kidney (FIG. 2D). The MIBI data showed several previously identified thymus-associated proteins were found to be expressed within the sTO, specifically IDO-1 and podoplanin, which have been demonstrated to be expressed in the thymic medulla, T-bet, which is a transcription factor linked to the olfactory bulb and thymus during embryogenesis, and TIM3, which has been associated with dendritic cells and other thymic cells (FIG. 2E, upper panel). CD4⁺ T-cells were also further characterized within a specific population of T cells within and around the sTOs (FIG. 2E, lower panel). Expression of these markers and T cells were absent in nongrafted mouse kidney examined as a comparison.

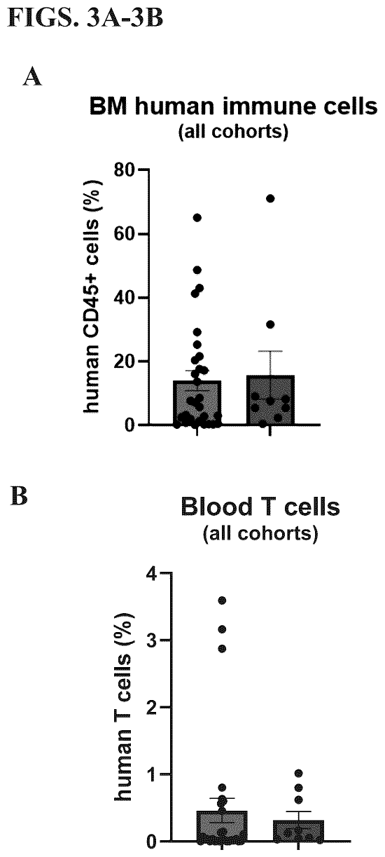

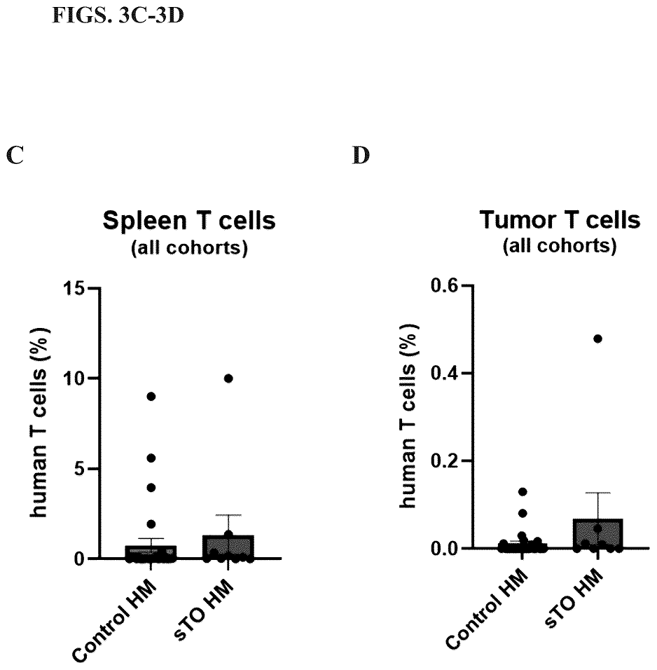

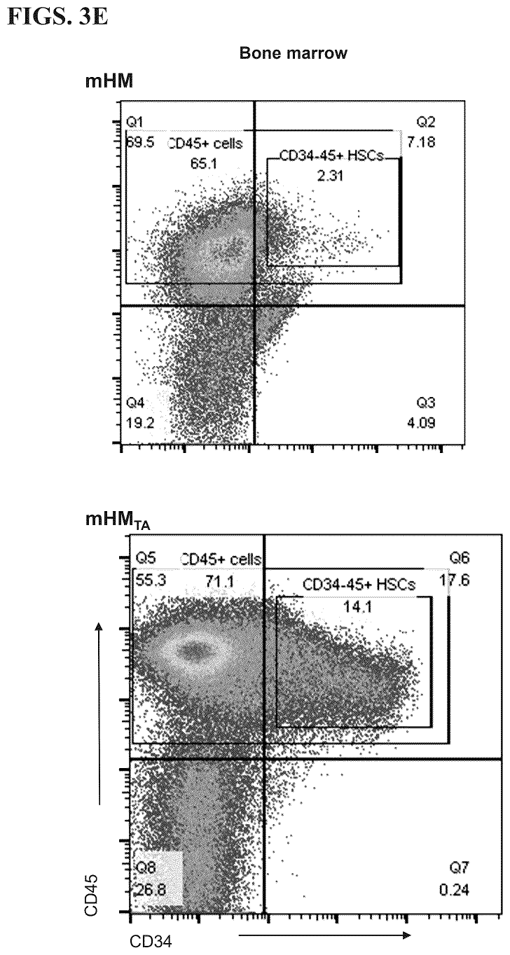

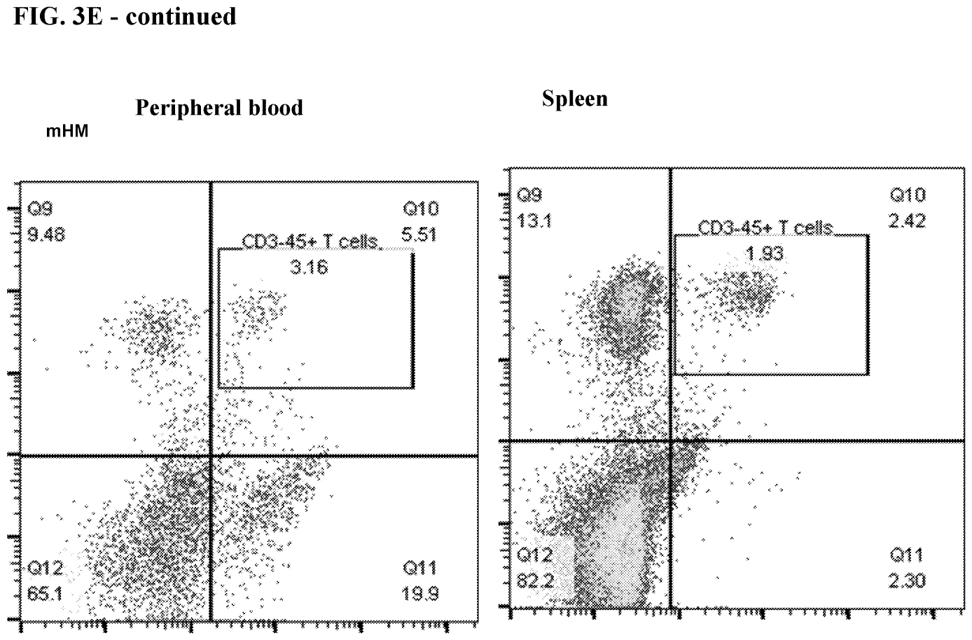

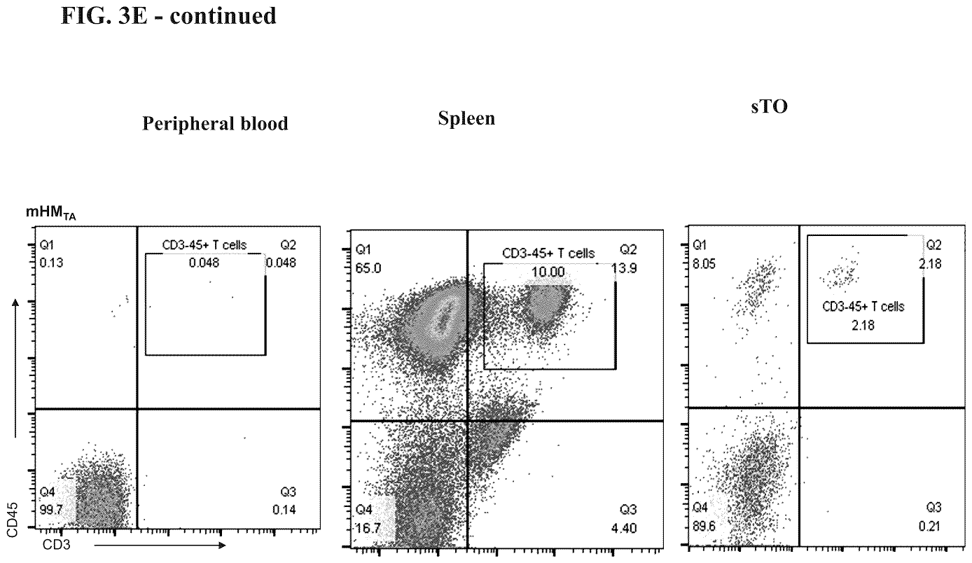

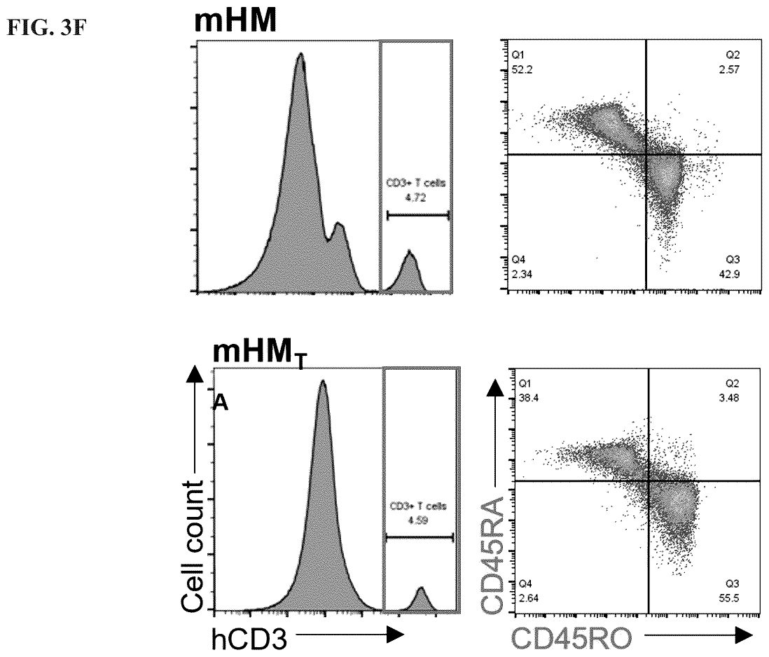

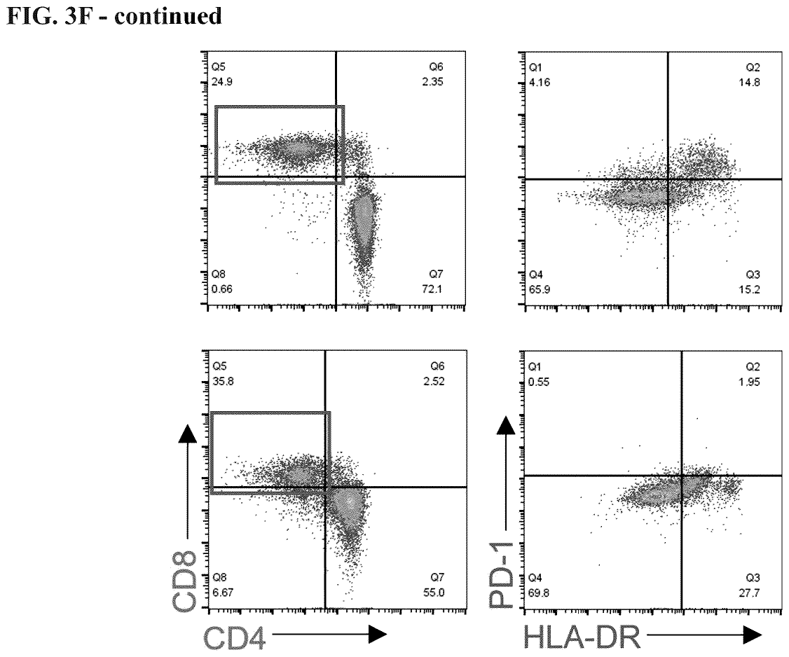

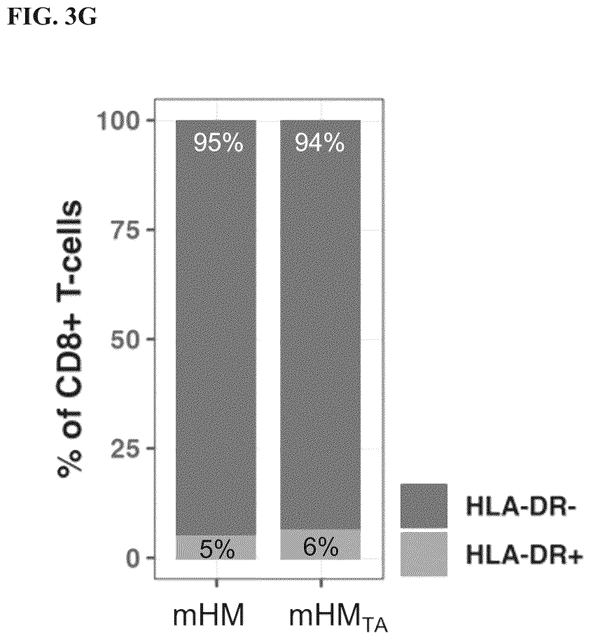

In yet another exemplary method, human CD45⁺ cells fractions were quantified in the bone marrow and CD3⁺ lymphocyte fractions were quantified in the blood, spleens, and tumors from the mHM (control HM) and mHM_{TA} (sTO HM) groups at the end of the study. There is no statistical difference between the two groups of animals, indicating that thymectomy and implantation of the sTO in the kidney capsule does not affect overall humanization of the mice (p=0.8077, unpaired T test). (FIG. 3A) Likewise, there was no statistical difference in the percentage of CD3⁺ T cells in the blood (p=0.6767, unpaired T test; FIG. 3B) or spleens (p=0.5229, unpaired T test; FIG. 3C) between the two groups. Graphical comparison of the tumor-infiltrating human CD3⁺ T cells identified in tumor tissue removed from control mHM and mHM_{TA} (FIG. 3D) suggest that T cells educated in the human thymic tissue of an implanted sTOs would be expected to more readily interact with tumor neoantigens, and infiltrate the tissue. Although at present, there is no statistical difference (p=0.0967), not all tissue from the sTO animals has been analyzed so a final calculation of statistical significance cannot be appropriately calculated. Flow cytometry showed that 71.1% of all lymphocytes in the bone marrow were of human origin and that there were distinct populations of T cells present in its peripheral blood (0.05%), spleen (10.0%), and sTO tissue (2.18%) (FIG. 3E).

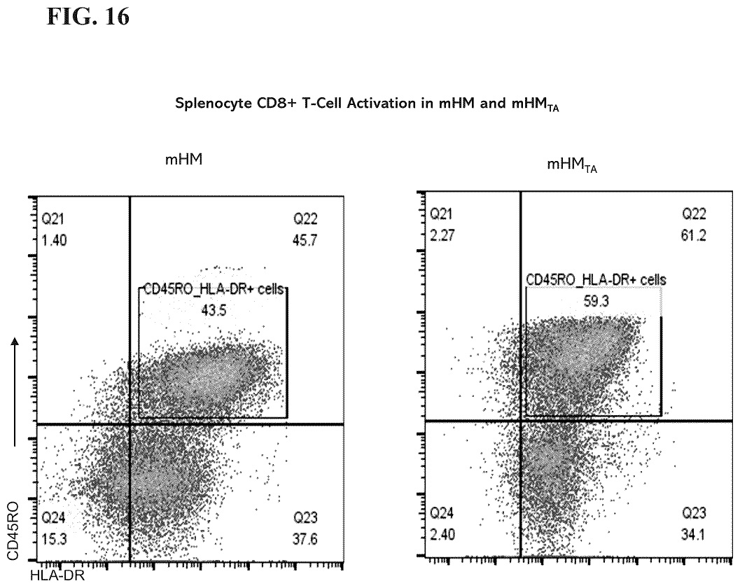

T cells in the spleen of mHM_{TA} were further analyzed and compared to with those identified in mHM and an increase of 12.9% CD45RO⁺/CD45RA⁺ T cell ratio in mHM_{TA} was observed, indicating that a greater percentage of mHM_{TA} T cells had interacted with a foreign antigen (FIG. 3F). Likewise, a greater percentage of the T cells in mHM_{TA} were CD8⁺ cytotoxic T cells (CD8⁺/CD4+=0.65; mHM mouse ratio=0.35), as might be expected in an HLA-A matched HM with an active anti-tumor immune response. Although the HLA-DR⁺ activated cells populations were similar, approximately half of these cells in mHM also expressed PD-1, indicating that they are functionally exhausted and likely not contributing to an active immune response. Conversely, there was very little PD-1 expression in the HLA-DR⁺ cells in mHM_{TA} spleen, indicating an increased potential for long-term activation (FIG. 3F bottom panel). Additionally, when T-cells were present, a greater percentage were CD45RO⁺/HLA-DR⁺ in mHM_{TA} (FIG. 16). Overall, HLA-DR+ T cells were present in slightly higher frequencies in mHM_{TA} (FIG. 3G), and these T-cells educated and maturing in the mHM_{TA} mice displayed features consistent with normal lymphoid development significantly different than those in the mHM.

### Example 4

sTO-Educated T Cells Inhibit Melanoma Growth

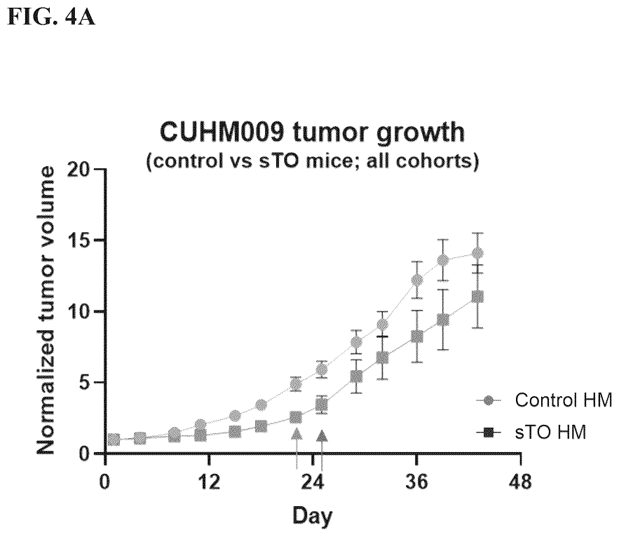

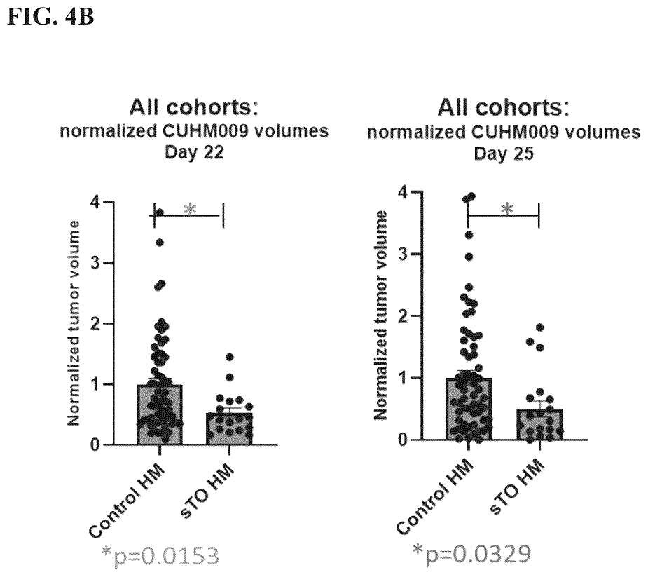

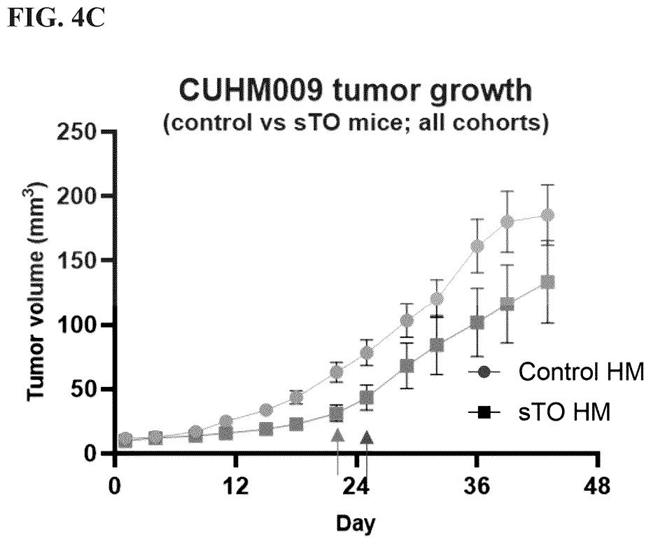

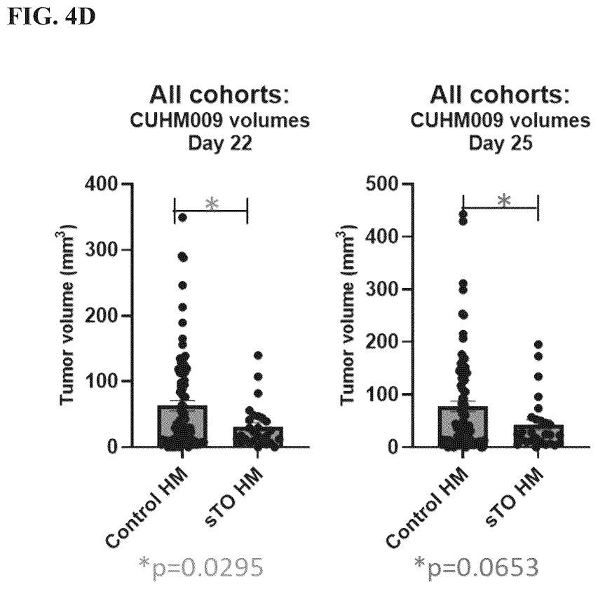

In another exemplary method, melanoma tissue, previously passaged on NSG mice as PDX and originating in the same patient who donated the PBMCs from which the sTOs were produced, was implanted on all mHM (control HM) and mHM_{TA} (sTO HM). The resulting tumors were measured on all mice to and averaged to compare tumor growth. (n=29 mHM, n=9 mHM_{TA}) When tumor sizes were normalized to their initial measurements and compared (FIG. 4A), tumors implanted on mHM_{TA} experienced a delay in growth, which became statistically significant on day 22 of the study. (p=0.0153, unpaired T test; FIG. 4B) This effect was also readily apparent when directly comparing tumor volumes (FIG. 4C), as tumors on the mHM_{TA} grew more slowly than those on the control mHM. (p=0.0295 on day 22, unpaired T test; FIG. 4D)

### Example 5

Tumor Growth Dynamics Change in mHM_{TA}

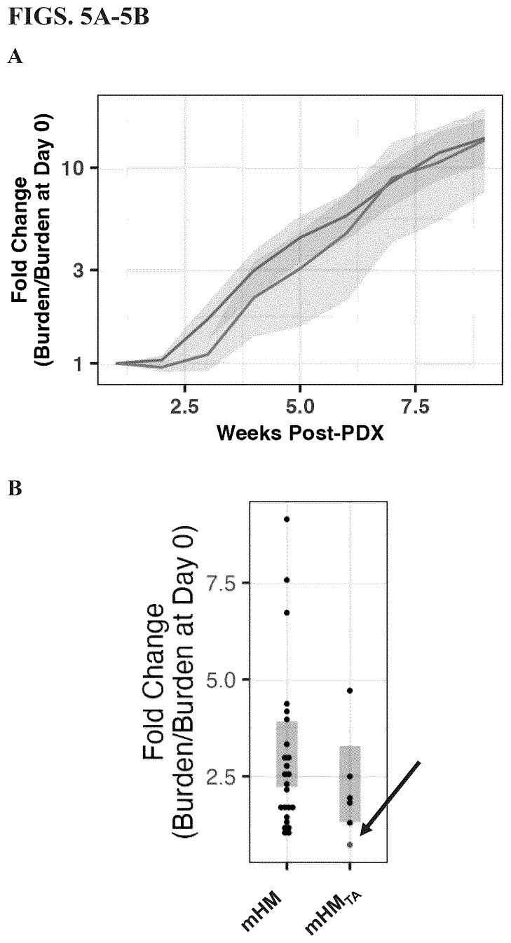

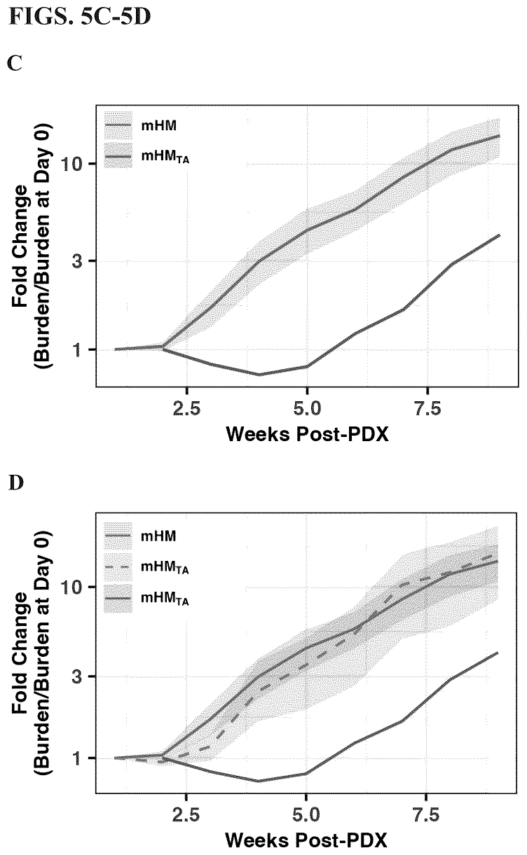

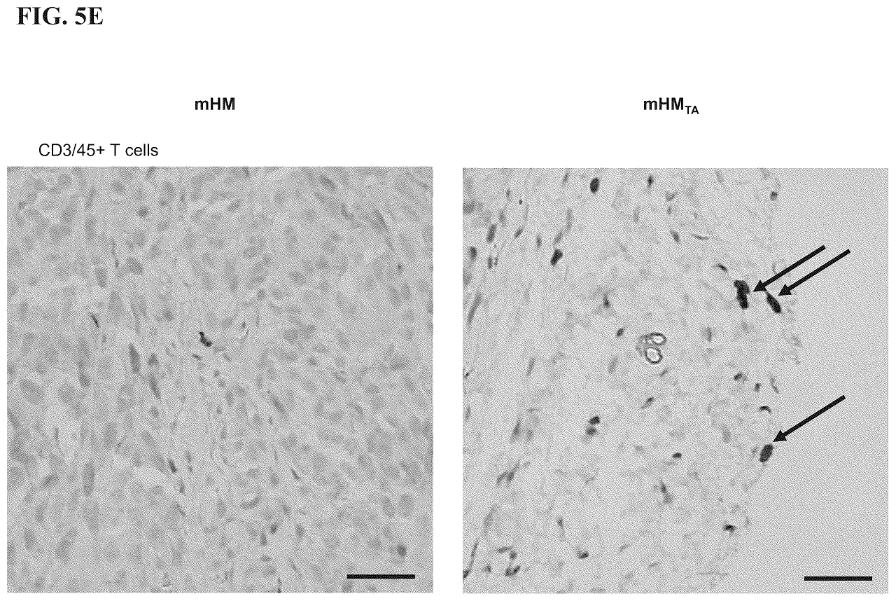

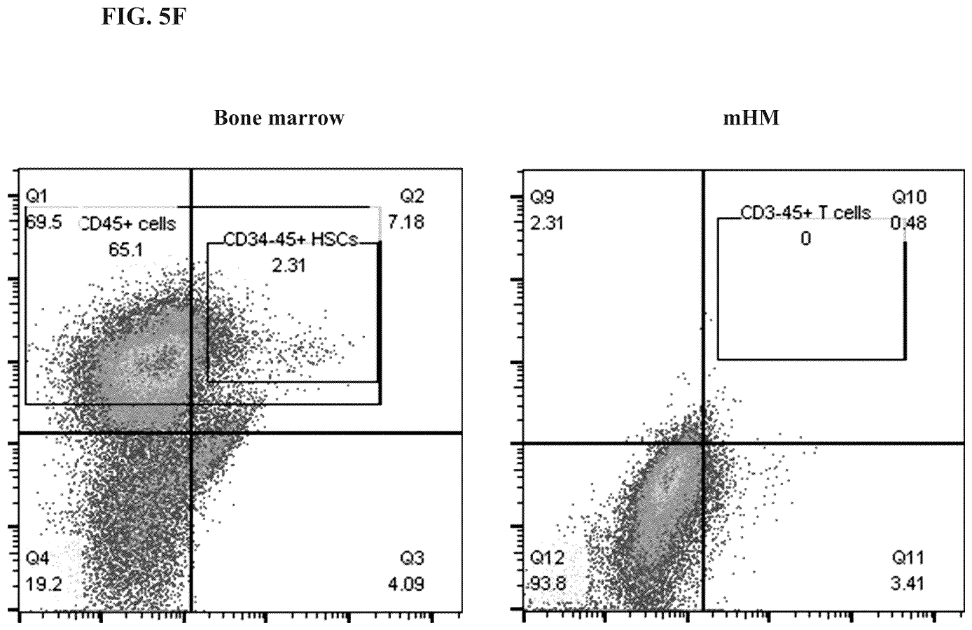

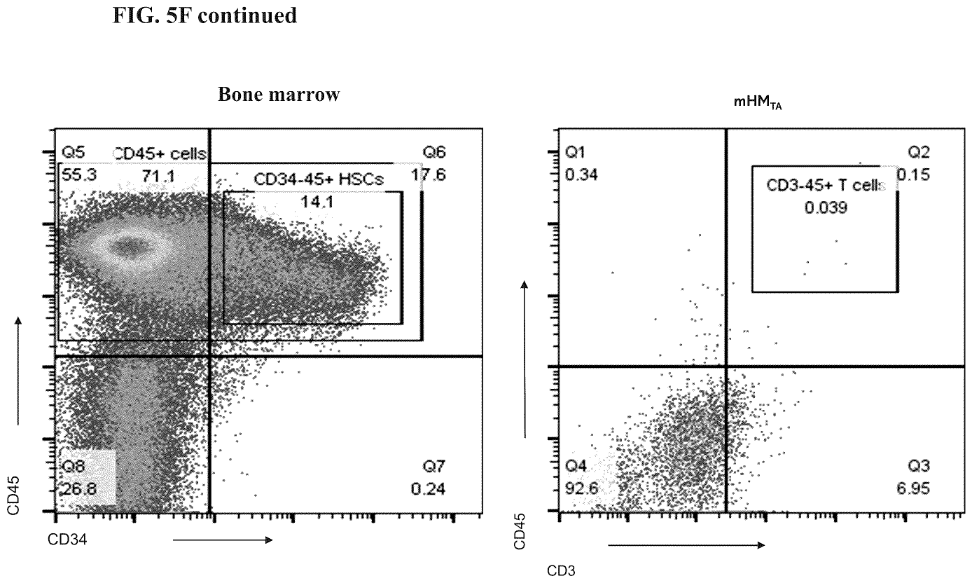

In further exemplary methods, PDX growth was measured in both mHM and mHM_{TA} models. It was hypothesized that T-cells maturing in sTOs autologous to the implanted tumor (mHM_{TA}) would be most effective in delaying tumor growth. Comparing tumor growth between the six mHM_{TA} mice vs mHM mice demonstrated a non-significant delay in tumor growth (FIG. 5A-B). To quantify the likelihood that the delayed tumor growth in the best mHM_{TA} mouse was due to chance, a Bayesian framework was used to conclude that there is moderate evidence that the T-cell producing sTO in this mHM_{TA} delayed tumor growth (FIG. 5C), which remains true regardless of how other sTO-engrafted mice were grouped (Bayes factor of 6.49) (FIG. 5D).

Further, T-cell populations within tumors implanted on mHM_{TA} were observed, concentrated within the tumor capsule (FIG. 5E), and tumor cytometry of the best mHM_{TA} and humanized mHM showed higher levels of T cell invasion of the tumor in mHM_{TA} (FIG. 5F). With evidence of delayed tumor growth and T-cell infiltration in the tumor capsule, further experiments were conducted to identify immunological characteristics that differentiated the tumor response in mHM_{TA} from that of mHM.

### Example 6

Single Cell Immune Profiling in Conjunction with Exome Sequencing of PDX Tumor Tissue Provides Insight into the Impact of the mHIMTA Model

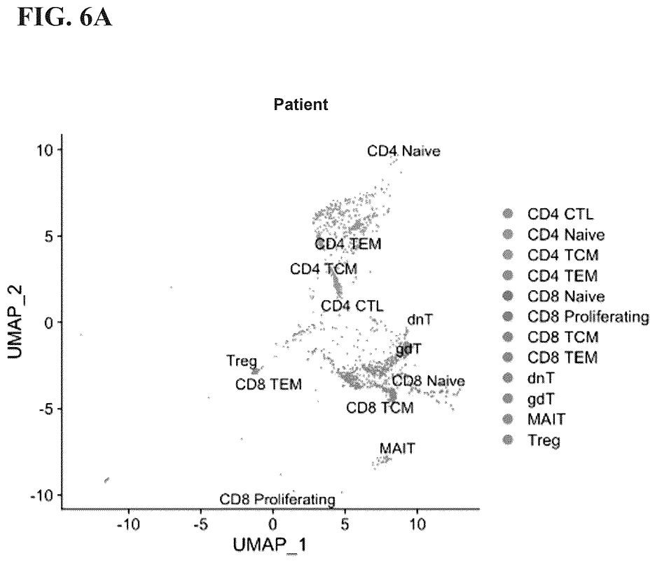

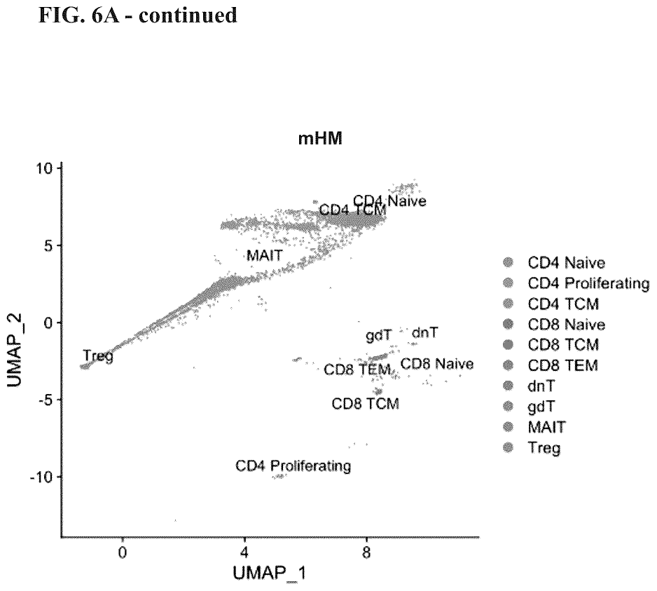

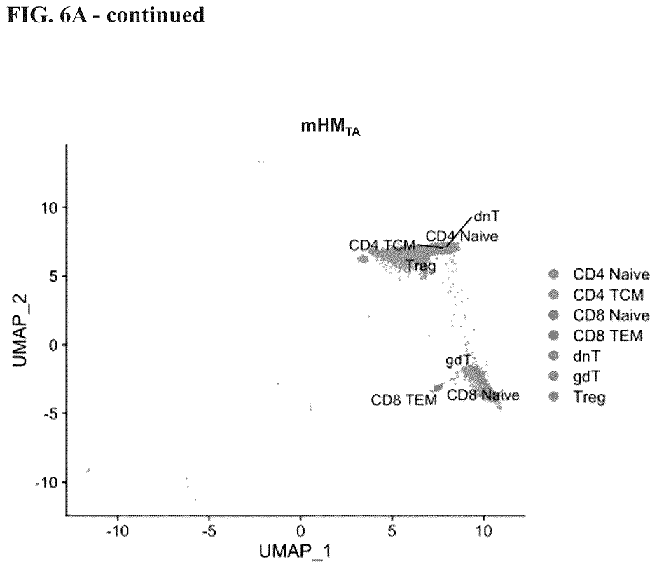

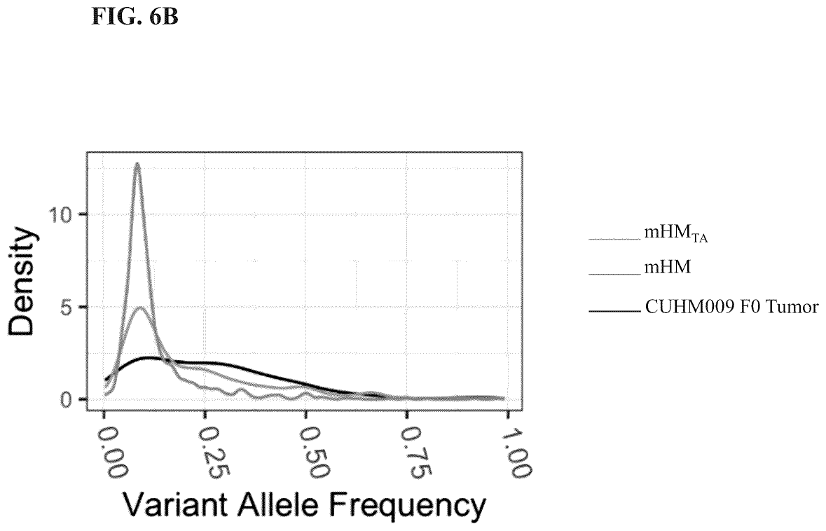

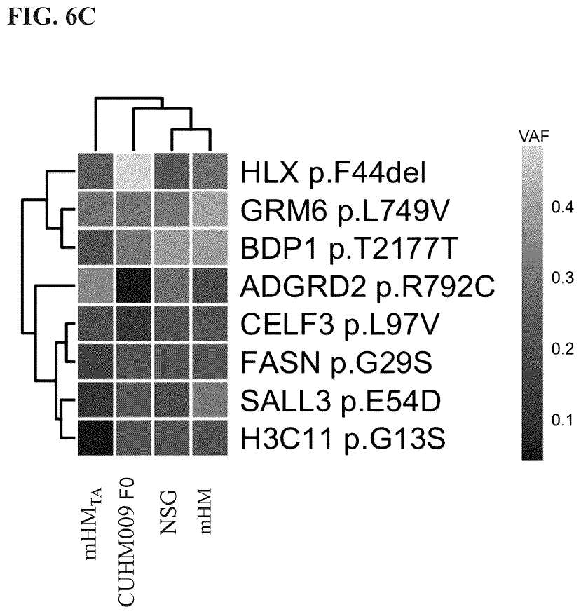

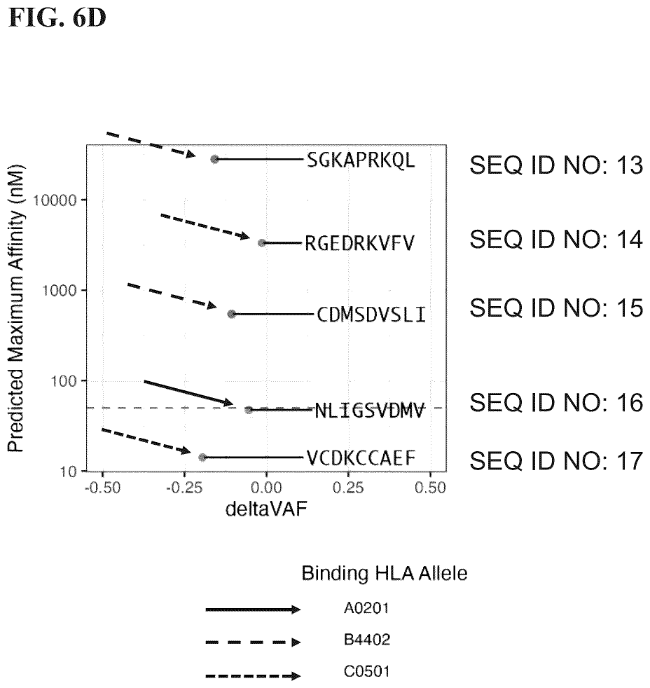

In additional exemplary methods, single cell sequencing of PBMCs from the patient (as benchmark), mHM, and mHM_{TA} mice demonstrated an abundance of regulatory T-cells (Tregs) in mHM that were not present in mHM_{TA} (FIG. 6A). Conversely, both naïve and memory effector CD8⁺ T cells (TEMs) were more abundant in the mHM_{TA}. Thymic availability enhances education and results in lasting immune memory.

To evaluate changes in intratumoral heterogeneity driven by varying immune permissiveness, high-depth exome sequencing of mHM_{TA} as well as mHM, NSG and F0 (originating patient) tumor tissue was performed. To limit sampling bias, half of each tumor was pulverized under liquid nitrogen and DNA was isolated from this homogenate to obtain DNA samples representative of spatially distinct and heterogenous cancer cell populations. The analysis was focused on somatic nonsynonymous protein coding variants that were detectable in the F0 sample. For each variant the variant allele frequency (VAF) was calculated as the fraction of reads supporting the variant allele to the total read depth at that locus. It was found that the distribution of variant allele frequencies (VAF) was significantly altered in the mHM_{TA} tumor tissue compared to that of mHM tissue (Kolmorogov-Smirnoff p<0.001) (FIG. 6B). To identify variants that were significantly perturbed between the mHM, mHM_{TA}, and F0 tumors, a binomial logistic regression model was fit at each variant locus and the intercept term was defined as the VAF value found in the parental tumor grown on immunodeficient NSG mice. Using least-squares means post-hoc testing, followed by Bonferroni correction for multiple tests, eight variants that demonstrated at least one significant difference between the mHM and mHM_{TA} experimental arms were identified (FIG. 6C).

In another exemplary method, change in VAF between mHM_{TA} and mHM was determined using the following equation: deltaVAF=VAF_{mHMTA}−VAF_{mHM}. According to this metric, variants with negative deltaVAF were less abundant in the mHM_{TA} population of cancer cells compared to that of mHM. From the eight variants that were significantly altered in between mHM and mHM_{TA} in the binomial logistic regression, the altered protein sequence was inferred for five peptides. In addition, putative neoantigens were identified using NetMHCpan HLA binding predictions of in silico digested mutant peptides. For each variant, the peptide sequence with the strongest affinity to any of the CUHM009 HLA alleles was identified. Incorporation of NetMHCpan predictions and the deltaVAF metric identified four high-affinity peptides (affinity <50 nM) and three of the four were less abundant in mHM_{TA} model (FIG. 6D). The neoantigens illustrated in the figure include SGKAPRKQL (SEQ ID NO: 13), RGEDRKVFV (SEQ ID NO: 14), CDMSDVSLI (SEQ ID NO: 15), NLIGSVDMV (SEQ ID NO: 16), and VCDKCCAEF (SEQ ID NO: 17).

### Example 7

In vitro patient-specific sTO characterization. Patient-derived iPSCs are differentiated into TEPs and subsequently converted to TECs. (FIG. 1A) Co-culture of allogeneic (for in vitro examination) or HLA matched HSPCs (for in vivo experiments) at the air-liquid interface produces sTOs with functional TECs. (FIG. 1B) Expression of these genes by qPCR of key thymic markers in sTO cultures at 0-, 3-, and 6-week timepoints was compared to that of undifferentiated iPSCs using their ΔΔCt values. (FIG. 1C) qPCR examination of TRA expression in vitro, indicative of AIRE activity, were calculated and with significance as determined above. Immunofluorescence microscopy of sTOs after three weeks of culture demonstrates cell structures expressing thymic markers and identifies presumptive T-cells within these structures. CD4⁺/CD8⁺ double positive T-cells are indicated by white arrows. (FIG. 1D) Magnification is 40×. Scale bar=100 μm. CD5⁺/7⁺ pro T-cells were identified by flow cytometry after gating CD45⁺ cells, while CD4⁺/8⁺ T-cells were identified after gating CD45⁺ and CD3⁺/TCRαβ cells. (FIG. 1E)

### Example 8

In vivo sTO characterization. Tumor biopsies were implanted as PDXs on NSG mice to expand prior to use in the HM. Patient blood was also drawn at the time of biopsy and reprogrammed into iPSCs and subsequent sTOs using HLA-A matched cord-blood derived HSPCs. The sTOs are surgically implanted into the kidney capsule of thymectomized mHM produced with these same HSPCs to produce the mHM_{TA} animals. After human peripheral blood cells have been identified in the HM, PDX tissue is passaged is into the mHM and mHM_{TA}. (FIG. 2A) A picture of a successfully engrafted sTO growing under the kidney capsule, indicated by the arrow, is illustrated in FIG. 2B. (Scale bar=1 mm.) Immunohistochemical comparison of human thymic tissue with engrafted sTO tissue demonstrates that the sTO tissue expresses both human KRT5 and KRT8. Magnification is 10×; scale bar=100 μm. (FIG. 2C, upper row) The magnified portion of the boxed region is illustrated in FIG. 2C, middle row. Columns 1-2, magnification is 20×; scale bar=50 μm. Column 3, magnification is 40×; scale bar=50 μm. Human thymus, for comparison, is shown in FIG. 2C, lower row. Magnification is 20×; scale bar=50 μm. Cytometry of a successfully engrafted sTO shows that the tissue harbors CD4⁺/CD8⁺ DP T-cells undergoing thymic selection and maturation. The presence of these T-cell populations is also observed by IHC. Magnification is 40×; scale bar=50 μm. (FIG. 2D) MIBI images of an sTO show that it specifically expresses the thymus-associated proteins IDO-1, Podoplanin, T-bet, and Tim3. (FIG. 2E, upper row) Their merged expression is illustrated in the final image. Images of sTO-associated T cells, initially showing an overview of the sTO and subsequently an example of a CD3⁺/CD4⁺ T cell enlarged in images are shown in FIG. 2E, lower row. The final image is of a nongrafted mouse kidney for comparison of all markers. White scale bar=100 μm. Red scale bar=25 μm.

### Example 9

Tumor growth analysis in the mHMTA. In another method, fold change in tumor burden from day 0 in mHM and mHM_{TA} was plotted on a log-transformed axis against study week with the bootstrapped mean and 95% confidence interval in gray. (FIG. 5A) Tumor growth was not significantly delayed in engrafted mHM_{TA} mice. Difference in tumor growth was assessed at day 30 using a Wilcox rank sum test (p=0.46). The red point (arrow) represents the mHM_{TA} mouse in which the most DP T cells were observed. (FIG. 5B) A successful mHM_{TA} mouse, illustrated in red, demonstrates a prolonged initial lag phase before progression when compared to all other mHM. (FIG. 5C) Using Bayesian methods, it was discovered that there was moderate evidence that the sTO in this mHM_{TA} delayed tumor growth when compared to any other mHM or mHM_{TA} (Bayes Factor 6.49). Using the same framework, it was found that there was strong evidence that the presence of sTO engraftment alone did not contribute to delayed tumor growth (Bayes Factor 8.3×10-16). (FIG. 5D) IHC of CD3⁺/CD45⁺ in tumors demonstrate T-cells infiltrating the mHM and mHM_{TA} tumors. (FIG. 5E) Magnification is 40×; scale bar=50 μm. (4F) Cytometry of the tumors of this mHM_{TA} and a comparably humanized mHM reveals the comparative infiltration of T cells. (FIG. 5F)

### Example 10

In another method, T-cell dynamics and their effect on tumor neoantigen selection. Single cell sequencing UMAP plots colored by Azimuth pipeline cell type predictions were generated. An abundance of Tregs were found in the mHM mice, whereas this population was lacking in the mHM_{TA} mice. (FIG. 6A) Deep exome sequencing of PDX tissue demonstrate that distribution of variant allele frequencies (VAFs) in the mHM_{TA} is substantially different than that of PDXs engrafted in the mHM model (KS test, p<0.001). (FIG. 6B) Binomial logistic modelling of VAFs identified eight subclonal variants that are consistent with immunoediting in the mHM_{TA} mouse model. mHM and mHM_{TA} variants with a significant marginal difference were plotted using a heatmap and ordered with hierarchical clustering and visualized against the F0 values. (FIG. 6C) Predicted neoantigens were generated by identifying significant changes between mHM and mHM_{TA} VAFs and plotted against the predicted HLA binding affinity for 9-mer peptides derived from each variant. (FIG. 6D) The points are grouped by which class I HLA molecule the peptide was predicted to bind. The dashed line represents a 50 nM affinity cutoff. Points below this line are considered strong binders.

### Example 11

iPSCs induction from patient PBMCs. In another example, FIG. 7A depicts an overview of iPSC generation. Patient blood is drawn and the PBMCs are isolated and reprogrammed into iPSCs as described herein. The TRA-1-60 and SOX2 expression of the CUHM009 iPSCs was compared to that of the previously characterized Mell-hESC cell line to verify successful reprogramming. (FIG. 7B)

### Example 12

T-cells are present in HSPCs used to produce sTOs. HSPCs added to the TEP cultures contain a small population of CD3⁺ T-cells, which increased throughout the six weeks of sTO culture and produced CD3⁺/TCRαβ⁺, CD4⁺, and CD8⁺ T-cells at roughly the same frequencies as observed in reference PBMCs. (FIG. 8A) During this period, the CD3⁺/CD45⁺ T-cells also express CD25, a marker of T-cell activation, with increasing frequency. (FIG. 8B)

### Example 13

Human lymphocytes in NSG, NSGs with sTOs, and thymectomized HM. Flow cytometry was performed on the blood of representative NSG, NSG+sTO, and mHM_{TA} mice. No T-cells are present in NSG and NSG with sTOs. (FIGS. 9A-9B)

### Example 14

Cord blood HSPC in vitro expansion. In another exemplary method, after expansion, cohort UCB120 HSPCs increased from an initial population of 790,800 to a final population of over 30×10⁶. Likewise, cohort UCB122 HSPCs increased from 646,000 to over 43×10⁶, enabling the generation of HM cohorts and HLA-A matched sTOs from each cord. (FIG. 10)

### Example 15

HSPC engraftment in HM bone marrow. In another embodiment, numbers of human CD45⁺ cells in all live cells isolated from HM bone marrow at the end of study was determined. There was no significant difference in the CD45⁺ fraction between mHM and mHM_{TA}, indicating that all groups were equally well humanized. (FIG. 11A) In addition, the number of human CD34⁺ HSPCs in all human CD45⁺ bone marrow cells indicated no differences between cohorts or treatment groups. Shaded error bars=95% CI by bootstrap. In both graphs, the formerly red point (arrow) represents the mHM_{TA} mouse in which the most CD34⁺/CD45⁺ double positive T cells were observed. (FIG. 11B)

### Example 16

Circulating human lymphocytes. In another method, number of human CD45⁺ cells among all live cells were calculated throughout the study, logarithmically transformed, and plotted for the mHM and mHM_{TA} groups. (FIG. 12) Lymphocytes increased at an equal rate in all cases. In another exemplary method, double positive T cell numbers were analyzed. The formerly red line (with the arrow) represents the mHM_{TA} mouse in which the most CD3⁺/CD45⁺ double positive T cells were observed.

### Example 17

Circulating human T-cells. In another method, number of human CD3⁺/CD45⁺ T-cells among all live cells was calculated throughout the study, logarithmically transformed, and plotted for all groups. (FIG. 13) The formerly red line (with the arrow) represents the mHM and mHM_{TA} in which the most CD3⁺/CD45⁺ double positive T cells were observed.

### Example 18

Characterization of additional examples of sTO engraftment. In another method, IHC analysis of additional sTOs identified in mHM_{TA}, demonstrated presence of human KRT5 and KRT8 proteins. (FIG. 14A) Magnification is 20×; scale bar=50 μm. The presence of CD3⁺/CD45⁺ was observed using flow cytometry. (FIG. 14B)

### Example 19

IHC of murine thymus expression. In another exemplary method, IHC of a representative murine thymus tissue from mHM demonstrated expression of only murine cytokeratins (KRT5 and KRT8). CD3⁺/CD45⁺ and CD4⁺/8⁺ double positive T-cells can also be observed within this tissue. (FIG. 15A) Magnification is 20×, scale bar=50 μm. Cytometry of the murine thymus tissue also positively verifies the presence of CD3⁺ T-cells within this tissue. (FIG. 15B)

### Example 20

Splenocyte CD8⁺ T-cell activation in mHM and mHM_{TA}. In another method, cytometric analysis of mHM and mHM_{TA} splenocytes indicates that a greater percentage are activated as assessed by CD45RO⁺/HLA-DR⁺ cells in the mHM_{TA} spleen. (FIG. 16)

### Methods

### Human and Animal Regulatory Approvals:

Studies involving human subjects were approved. Written informed consent was obtained from the patient whose blood and tumor tissue were used for this study (CUHM009). Animal procedures were approved. PDX generation and animal care have been previously reported.

### sTO Generation

The collection and isolation of patient HSPCs and PMBCs has been previously described. Induction of iPSCs and subsequent differentiation to TEPs was conducted as previously described, with slight modifications: at approximately day 18-31, TEPs were harvested using a 1 mL pipet, mechanically dissociated into small clumps, and transferred to a 1.5 mL tube.

For initial sTO characterization, allogenic HSPCs were isolated by magnetic-activated cell sorting (Miltenyi Biotec) from UCB units previously obtained from the University of Colorado cord blood bank. These HSPCs were added to CUHM009 patient-derived TEPs, and the suspensions were spun down at 1200 RPM for 3 minutes. The cell slurry was plated in 5 μL drops onto a 0.4 μm Millicell Transwell insert (Millipore Sigma, Darmstadt, Germany) to form 2-3 sTOs plated per membrane. Transwells were placed in a 6-well plate containing 1.5 mL of RB27 media as previously described, substituting SMI without vitamin A (Stemcell Technologies, Vancouver, BC) for B27. A 2-Phospho-L-ascorbic acid trisodium salt (Sigma-Aldrich, St. Louis, MO;) was added at 50 μg/mL. Half of the media was changed every 2-3 days. sTOs were cultured in this manner for up to 6 weeks before harvest.

For the in vivo experiments, the same HLA-A matched HSPCs employed to humanize the mice were combined with CUHM009 patient-derived TEPs at an approximate 1:20 ratio, and the mixture was centrifuged at 1200 RPM for 3 minutes, supernatant was removed, and 15 μL aliquots were implanted into the kidney capsules of the HM. Representative sTOs were harvested approximately 10 weeks after implantation for analysis.

### sTO Characterization

For in vitro experiments: Flow cytometry was performed on a Cytek Aurora flow cytometer (Cytek, Fremont, CA) to characterize (1) pro-T-cells using CD34, CD7, and CD5 antibodies (Biolegend, San Diego, CA;, 343114, 364009) at 1:20, 1:60, and 1:40, respectively, and (2) T-cells using CD3, CD4, CD8, CD45, and TCRα/β antibodies (Biolegend, Cat #344806, 300520, 300916, 304025, and 306720) at 1:100, 1:100, 1:100, 1:180, and 1:60, respectively. For qPCR analysis, reverse transcription of RNA was performed using an iScript cDNA synthesis kit (Bio-Rad Laboratories, Hercules, CA). For IF, 4-10 μm tissue sections were cut from paraffin or OCT-embedded tissue blocks. Deparaffination and antigen retrieval have been previously described. (See, for example, Ramos S A et al., J Allergy Clin Immunol, 2021 and Ramos S A et al., Stem Cell Reports, 2023, both incorporated herein by reference in their entireties for all purposes). Slides were stained using the following antibodies and dilutions: CD3 (Abcam, Cambridge, UK) 1:100, CD4 (Santa Cruz Biotechnology, Dallas, TX;) 1:100, CD8 (Abcam) 1:100, EPCAM (BioLegend) 1:200, KRT5 (Abcam) 1:100, KRT8 (Santa Cruz) 1:100, panKRT (Abcam) 1:300, PDGFRα (Abcam) 1:500, The Z-stack or snap images were taken with a Zeiss LSM 800 microscope (Carl Ziess AG, Oberkochen, Germany).

### HSPC Collection, Expansion, and Engraftment in Mice and Renal Capsule sTO Implantation

HSPC purification, expansion, and engraftment were conducted as previously described (Morton J J et al., Mol Carcinog 2018). Two independent cohorts of HM were generated for this study using HSPCs derived from donor umbilical cord blood. One week after humanization, sTOs were implanted in selected HM and NSG controls. Animals were anesthetized with isoflurane. A 15 μL aliquot sTO cell slurry (equaling material from approximately six sTOs mixed with 1.2×10⁶ HSPCs from those expanded to humanize the mice) was injected under the kidney capsule, as described previously (Faleo G. et al., Stem Cell Reports 2017; 9:807-19). Mice were considered eligible for analysis if human thymic tissue was detected in the renal capsule post-mortem.

### Fluorescence Activated Cell Sorting (FACS) and Flow Cytometry

Tumor and mouse tissues were prepared for cytometric analysis as previously described. Cell sorting was performed using a MoFlo XDP70 (Beckman Coulter, Brea, CA), and flow cytometry was completed on a ZE5 (Bio-Rad Laboratories) or an Aurora Spectral Analyzer (Cytek, Fremont, CA) and analyzed using FlowJo software (Becton Dickinson, Franklin Lakes, NJ). In addition to those antibodies mentioned above, human blood cells were identified by CD4, CD8, PD1, CD14, CD45RA, and CD45RO (e.g., Becton Dickinson) antibodies, 1:10 dilution. Mouse CD45 (e.g., Biolegend) antibody was used to exclude murine leukocytes.

### Immunohistochemistry (IHC) and Multiplexed Ion Beam Imaging (MIBI)

IHC analyses were performed as described (Morton J J et al., Oncogene 2016; 35:290-300). Samples were submitted to the Human Immune Monitoring Shared Resource at the University of Colorado School of Medicine for MIBI analysis using their validated antibody panel. Stained slides were analyzed using MIBItracker software (Ionopath, Menlo Park, CA).

### Single Cell Sequencing

Cells were sorted using a MoFlo XDP70 then submitted to the Genomics Shared Resource at the University of Colorado Cancer Center for single cell capture and gene expression library preparation using the 10× Genomics Chromium X system using the Chromium Next GEM Automated Single Cell 5′ Kit v2. Libraries were then sequenced on a NovaSeq6000 instrument using an S4 flow cell with v1.5 chemistry.

### Whole Exome Sequencing

Genomic DNA samples were submitted to the Genomics Shared Resource at the University of Colorado Cancer Center. Following quantitation using a Qubit Fluorometer (ThermoFisher), 200 ng of genomic DNA from each sample was used for library preparation following the manufacturer's protocol. For whole-exome sequencing (WES) library preparation, the SureSelect Human All Exon V6 r2 (Agilent Technologies, Santa Clara, CA; Cat #S07604514) target enrichment kit was used with the Agilent SureSelectXT system. Libraries were sequenced on an Illumina NovaSeq6000 Instrument, using an S4 flow cell with v1.5 chemistry.

### NGS Data Analysis and Bioinformatics

A pipeline was constructed modeled from the GATK Best Practices for Somatic Variant Calling. Adapter sequences were trimmed using the Agilent Genomics NextGen Toolkit (AGENT) trim module. Reads were then aligned to GRCh38.p13 (RefSeq assembly accession: GCF_000001405.39) using BWA as implemented in BWA kit (github.com/lh3/bwa/tree/master/bwakit). For single cell sequencing, reads were processed using the cellranger count pipeline (10× Genomics, Pleasanton CA).

### Statistics

In vitro and in vivo (using ≥5 mice/group) experiments were compared with Brown-Forsythe ANOVAs and two-sided t tests. Calculations were done using GraphPad Prism version 8.3. Data are represented graphically as mean±SEM. GSEA estimates the statistical significance of the enrichment scores by a two-sided modified Kolmogorov-Smirnov permutation test. P and Q values of less than 0.05 were statistically significant. All statistical tests were two-sided.

sTO analysis

For flow cytometry analysis, sTOs were harvested by adding 0.5 mL of FACS buffer (0.1% BSA and 2 mM EDTA in PBS) to each membrane, mechanically dissociating the sTOs, and passing the sTO suspensions through a 40 μm FACS filter into a 5 mL round bottom polystyrene FACS tube. For qPCR analysis, sTOs were harvested with 0.5 mL FACS buffer, transferred to a 1.5 mL tube, and pelleted by centrifugation. The cell pellet was resuspended in 350 μL of Qiagen buffer RLT lysis buffer (Qiagen, Germantown, MD, Cat #79216), stored at −80 °C, and RNA was isolated using a RNeasy Mini Kit (Qiagen, Cat #74104) per manufacture instructions. To prepare sTOs for Immunofluorescence (IF), 4% PFA was added, and sTOs were fixed for 15 minutes at room temperature. The sTOs were washed three times with PBS, which was then replaced with 30% sucrose, and stored overnight at 4 °C sTOs were then embedded in OCT Compound (Leica Biosystems, Wetzlar, Germany, Cat #39475237) in a sectioning mold, snap frozen, and stored at −80 °C.

### sTO Characterization

Real-time quantitative PCR was performed on a CFX96 Touch Real-Time PCR Detection System (Bio-Rad Laboratories) using human-specific Taqman probes (Bio-Rad Laboratories or ThermoFisher Scientific, Waltham, MA) or human-specific primers listed below. Samples were normalized to endogenous control gene ACTB and plotted relative to undifferentiated iPSCs.

TABLE 2

| Probe | Supplier: Assay | Primer | | |
|---|---|---|---|---|
| Target | ID | Name | Sequence | SEQ ID NO: |
| ACTB | ThermoFisher: | ACTB | CATGTACGTTGCTATCCAGGC | 1 |
| | Hs01060665_g1 | For | | |
| | | ACTB | CTCCTTAATGTCACGCACGAT | 2 |
| | | Rev | | |
| ACTB | ThermoFisher: | Pro- | GCAGCCTTTGTGAACCAACAC | 3 |
| | Hs99999903_m1 | insulin | | |
| | | For | | |
| | | Pro- | CCCCGCACACTAGGTAGAGA | 4 |
| | | insulin | | |
| | | Rev | | |
| AIRE | Bio-Rad: | Islet | CGGGACACATGATTCTGGCAT | 5 |
| | qHsaCIP0029272 | Antigen | | |
| | | 2 For | | |
| | | Islet | CTGCTTGGTAGGCACAGAGG | 6 |
| | | Antigen | | |
| | | 2 Rev | | |
| DLL4 | Bio-Rad: | GAD1 | GCGGACCCCAATACCACTAAC | 7 |
| | qHsaCEP0051500 | For | | |
| | | GAD1 | CACAAGGCGACTCTTCTCTTC | 8 |
| | | Rev | | |
| FOXN1 | ThermoFisher: | MBP | GGCCGGACCCAAGATGAAAA | 9 |
| | Hs00919266_m1 | For | | |
| | | MBP | CCCCAGCTAAATCTGCTCAGG | 10 |
| | | Rev | | |
| HLA- | Bio-Rad: | TG For | AGACACCTCCTACCTCCCTCA | 11 |
| DRA | qHsaCEP0040019 | TG Rev | TCCTTGGACATCGCTTTGGC | 12 |
| KRT5 | Bio-Rad: | | | |
| | qHsaCEP0055058 | | | |
| KRT8 | Bio-Rad: | | | |
| | qHsaCEP0041467 | | | |

### Mouse Thymectomies

Prior to humanization, cohorts of mice were thymectomized. Surgery was slightly modified from a previously published procedure (Khosravi-Maharlooei M et al., Eur J Immunol 2020; 50:138-41). Briefly, six-week NSG mice were induced with ketamine/xylazine and intubated using a RoVent small animal ventilator (Kent Scientific, Torrington, CT). The ventilator was set to maintain a small positive expiratory end pressure (PEEP) to prevent pneumothorax once the chest wall was opened. Once stably intubated, mice were given 0.05 mL buprenorphine SR (Ethiqa; Fedelis Animal Health, North Burnswick, NJ). A surgical anesthesia plane was achieved and maintained using isoflurane. Mice were positioned dorsally on an infrared heating pad with homeostatic control. A longitudinal incision measuring approximately 1 cm and centered at the sternal notch was made, exposing the submandibular salivary gland. The salivary gland was moved rostral and supported by a moistened gauze sponge to prevent desiccation. An English nail anvil was used to divide the sternum from the sternal notch to the second rib. The strap muscles were then bluntly divided longitudinally to expose the mediastinum. The thymus was grasped with corneal forceps and removed with micro scissors. The specimen was fixed in formalin and assessed by IHC. The chest wall was closed with 6-0 polyglactin to reapproximate the sternum, followed by 6-0 silk suture for the skin. Animals were weaned from the ventilator over the course of 30 minutes, or until spontaneous respirations occurred. If no spontaneous respirations occurred, Dopram (Baxter Pharmaceuticals, Deerfield, IL) was administered. Animals were then allowed to recover on a warming blanket then monitored daily until complete recovery.

### HSPC Purification, Expansion, and Engraftment

HSPCs were purified from either cord or patient blood by CD34⁺ positive cell selection (Stemcell Technologies), suspended in serum-free expansion medium (Stemcell Technologies), and cultured at 37 °C, 5% CO₂ for 5-8 days. Cells were characterized by cytometry, using CD34, CD45, CD73, and CD166 antibodies (Biolegend) at a 1:10 dilution. NSG (Jackson Laboratories, Bar Harbor) mice were primed for engraftment by 1.5 Gy whole-body irradiation. After a recovery of 4-6 hours, the mice were each injected with 400,000 expanded CD34⁺ cells, suspended in 0.2 mL sterile PBS. When present, MSC-like cells were added to include 5% of the total injected cells. The mice were bled via the tail vein every two weeks to assess HSPC engraftment. Their peripheral blood was analyzed by flow cytometry, using human CD3, CD11b or CD14, CD19 and/or CD45 (Biolegend) antibodies 1:10.

### IHC Analysis

Primary antibodies and dilutions: CD45 (Dako), 1:100; CD3 (Abcam; San Francisco, CA), 1:500; hKRT5 (ThermoFisher), 1:200; hKRT8 (Abcam), 1:500; Ki67 (ThermoFisher), 1:100. Staining was developed using the following conditions: En Vision+Dual Link System HRP (Dako) for 30 minutes and substrate-chromogen (DAB+) Solution (Dako) for 5 minutes. Slides were then counterstained with Automated Hematoxylin (Dako) for 10 minutes.

### Single Cell Sequencing

Cryopreserved PBMCs from humanized mice and patient blood were thawed at 37 °C, then allowed to recover in complete RPMI1640 media (ThermoFisher, Cat #11875093) for one hour. The cells were then stained with PE conjugated anti-Human CD45 Clone HI30 (Biolegend, Cat #304039) then washed and suspended in 0.04% recombinant human Albumin supplemented HBSS. DAPI was added at a concentration of 3 UM for dead cell exclusion. CD45⁺/DAPI cells

### Whole Exome Sequencing

Samples of tumor tissue were flash-frozen in liquid nitrogen at the time of tissue collection, and stored at −80 °C. For genomic DNA extraction, the frozen tissue was ground with a mortar and pestle under liquid nitrogen. The pulverized tissue was digested with Proteinase K for 1 hour at 56 °C and DNA was isolated using phenol-chloroform and ethanol precipitation using established methods. CUHM009 DNA isolates were overtly contaminated with melanin, a PCR inhibitor. To clean up the CUHM009 DNA samples and avoid potential confounding variables all DNA samples were treated with the OneStep PCR Inhibitor Removal Kit (Zymo Research, Irvine, CA).

### Genomic Sequencing and Data Analysis

A combination of the HLA genotype calls from BWA kit and high-resolution HLA typing results provided by the Barbara Davis Center for Childhood Diabetes HLA Laboratory were used to obtain HLA types. Following alignment, PCR and optical sequencing duplicates were removed using Picard MarkDuplicates. Base quality scores were recalibrated using the BaseQualityRecalibrator tool and ApplyBQSR. Somatic variants were called using Mutect2 in multi-sample tumor-normal mode with each patient and the PDXs derived from their tumor representing one cohort. Genomic DNA from PBMCs was used for the normal sample in each case. Variants were annotated using GATK Funcotator and filtered for high confidence calls. Somatic non-synonymous variants were identified, and the mutant amino acid sequence was inferred from the resulting mutation. A sliding window approach was used to produce in-silico “digested” nine amino acid length peptides that incorporated the variant region. Using NetMHCpan-4.1, variant peptides' affinities for each class-I HLA molecule within the patient's HLA genotype was predicted.

All the compositions and methods disclosed and claimed herein may be made and executed without undue experimentation in light of the present disclosure. While the compositions and methods have been described in terms of preferred embodiments, it will be apparent to those of skill in the art that variation may be provided to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A humanized mouse model comprising:
engrafted human stem-cell derived thymic organoids (sTOs) and at least one engrafted human tissue sample, wherein a source of the human stem-cells from which the sTOs are derived and a source of the at least one engrafted human tissue sample are the same or are HLA-matched, and the humanized mouse is HLA-matched to the sTOs.

2. The humanized mouse model according to claim 1, wherein the at least one engrafted human tissue sample comprises at least one of human cells and human tissue.

3. The humanized mouse model according to claim 2, wherein the at least one engrafted human tissue sample comprises a human patient and the human patient has cancer, an autoimmune disorder, an immunocompromising disorder, another disorder, or a combination thereof.

4. The humanized mouse model according to claim 2, wherein the human patient has cancer, and the cancer comprises a solid tumor or a non-solid tumor.

5. The humanized mouse model according to claim 4, wherein the cancer is capable of metastasizing or has metastasized.

6. The humanize mouse model according to claim 4, wherein the cancer comprises skin cancer, head and neck cancer, liver cancer, brain cancer, breast cancer, lung cancer, mesothelioma, thymoma, bladder cancer, kidney cancer, testicular cancer, prostate cancer, pancreatic cancer, cholangiocarcinoma, stomach cancer, esophageal cancer, small intestine cancer, colon or colorectal cancer, peritoneal cancer, ovarian cancer, fallopian tube cancer, endometrial cancer, cervical cancer, sarcoma, leukemia, lymphoma, myeloma, myeloma, myeloproliferative disorders, neuroendocrine cancer, undifferentiated cancers, and cancers of unknown origins.

7. The humanized mouse model according to claim 4, wherein the cancer is melanoma.

8. The humanized mouse model according to claim 3, wherein the engrafted at least one of cells and tissue sample of the human patient comprises at least one of cells and tissue sample from a tumor of the human patient.

9. The humanized mouse model according to claim 1, wherein the HLA matched to the sTOs is derived from a patient or another subject.

10. The humanized mouse model according to claim 1, wherein the sTOs are prepared from peripheral blood mononuclear cells (PBMCs) from a patient.

11-16. (canceled)

17. The humanized mouse according to claim 1, wherein the humanized mouse comprises at least one of an immunocompromised mouse and a thymectomized mouse.

18-19. (canceled)

20. The humanized mouse according to claim 1, wherein the humanized mouse of the humanized mouse model is engrafted with the at least one of cells and a tissue sample from a tumor of a human subject having cancer, wherein the cancer comprises one or more of skin cancer, head and neck cancer, liver cancer, brain cancer, breast cancer, lung cancer, mesothelioma, thymoma, bladder cancer, kidney cancer, testicular cancer, prostate cancer, pancreatic cancer, cholangiocarcinoma, stomach cancer, esophageal cancer, small intestine cancer, colon cancer, peritoneal cancer, ovarian cancer, fallopian tube cancer, endometrial cancer, cervical cancer, sarcoma, leukemia, lymphoma, myeloma, myeloma, myeloproliferative disorders, neuroendocrine cancer, undifferentiated cancers, and cancers of unknown origins.

21. The humanized mouse according to claim 1, wherein the sTOs and the engrafted at least one of cells and a tissue sample of a patient are from the same patient.

22-26. (canceled)

27. The humanized mouse according to claim 1, wherein the sTOs of the humanized mouse comprise CD3+ or CD4+/ CD8+ double or single positive T-cells.

28. The humanized mouse according to claim 1, wherein the sTOs express one or more thymic markers comprising EPCAM, KRT5, KRT8A, or a combination thereof.

29-30. (canceled)

31. A method for generating a humanized mouse according to claim 1, comprising:
introducing sTOs to an HLA-matched mouse that is at least one of immunocompromised and thymectomized mouse.

32. The method according to claim 31, further comprising engrafting the sTOs into the humanized mouse.

33. (canceled)

34. A cell or tissue isolated from the humanized mouse according to claim 1.

35. A method for evaluating efficacy of a therapeutic agent to treat a patient having a health condition, comprising administering the therapeutic agent to a humanized mouse model according to claim 1; and determining efficacy of the at least one therapeutic agent to treat the health condition,
wherein the humanized mouse comprises stem-cell derived thymic organoids (sTOs) from a mammalian subject, wherein the humanized mouse comprises a mouse HLA-matched to the sTOs, and further comprises at least one of engrafted cells and a tissue sample, wherein the engrafted at least one cell and tissue sample is from a patient HLA-matched to the sTOs.

36. The method according to claim 35, wherein the health condition comprises at least one of cancer, an autoimmune disorder, an immunocompromising disorder, or other disorder.

37-42. (canceled)

43. A kit for generating a humanized mouse model according to claim 1 comprising,
a composition comprising stem-cell derived thymic organoids (sTOs); a cell or tissue sample HLA matched to the sTOs; and at least one container.

44-46. (canceled)

47. A method of treating a human subject having a health condition comprising,
creating a humanized mouse model according to claim 1 comprising obtaining stem-cell derived thymic organoids (sTOs) from the human subject, wherein the humanized mouse comprises at least one of immunocompromised and thymectomized mouse,
engrafting at least one of a cell and tissue sample from the patient to the mouse, wherein the engrafting is performed before, concurrently with, or after introduction of the STOs;
administering at least one therapeutic agent to the humanized mouse wherein the at least one therapeutic agent comprises at least one therapeutic compound used for treating a health condition or inducing an immune response in the patient;
assessing efficacy of the at least one therapeutic agent to treat the health condition, induce an immune response, and/or reduce a symptom of the health condition using the humanized mouse; and
administering to the patient the at least one therapeutic agent that demonstrates efficacy to prevent, treat, ameliorate, or eliminate the health condition, induces an immune response, or reduces a symptom of the health condition.

48-52. (canceled)


SEQUENCE LISTING (19 sequences)

SEQ ID NO: 1 - DNA, 21 bp, synthetic construct
  source 1..21: mol_type = other DNA
catgtacgttgctatccaggc

SEQ ID NO: 2 - DNA, 21 bp, synthetic construct
  source 1..21: mol_type = other DNA
ctccttaatgtcacgcacgat

SEQ ID NO: 3 - DNA, 21 bp, synthetic construct
  source 1..21: mol_type = other DNA
gcagcctttgtgaaccaacac

SEQ ID NO: 4 - DNA, 20 bp, synthetic construct
  source 1..20: mol_type = other DNA
ccccgcacactaggtagaga

SEQ ID NO: 5 - DNA, 21 bp, synthetic construct
  source 1..21: mol_type = other DNA
cgggacacatgattctggcat

SEQ ID NO: 6 - DNA, 20 bp, synthetic construct
  source 1..20: mol_type = other DNA
ctgcttggtaggcacagagg

SEQ ID NO: 7 - DNA, 21 bp, synthetic construct
  source 1..21: mol_type = other DNA
gcggaccccaataccactaac

SEQ ID NO: 8 - DNA, 21 bp, synthetic construct
  source 1..21: mol_type = other DNA
cacaaggcgactcttctcttc

SEQ ID NO: 9 - DNA, 20 bp, synthetic construct
  source 1..20: mol_type = other DNA
ggccggacccaagatgaaaa

SEQ ID NO: 10 - DNA, 21 bp, synthetic construct
  source 1..21: mol_type = other DNA
ccccagctaaatctgctcagg

SEQ ID NO: 11 - DNA, 21 bp, synthetic construct
  source 1..21: mol_type = other DNA
agacacctcctacctccctca

SEQ ID NO: 12 - DNA, 20 bp, synthetic construct
  source 1..20: mol_type = other DNA
tccttggacatcgctttggc

SEQ ID NO: 13 - protein, 9 aa, synthetic construct
SGKAPRKQL

SEQ ID NO: 14 - protein, 9 aa, synthetic construct
RGEDRKVFV

SEQ ID NO: 15 - protein, 9 aa, synthetic construct
CDMSDVSLI

SEQ ID NO: 16 - protein, 9 aa, synthetic construct
NLIGSVDMV

SEQ ID NO: 17 - protein, 9 aa, synthetic construct
VCDKCCAEF

SEQ ID NO: 18 - protein, 11 aa, synthetic construct
  MOD_RES 10: note = phosphorylated serine
  MOD_RES 11: note = C-terminal -NH2
KEAPPAPPQSP

SEQ ID NO: 19 - protein, 12 aa, synthetic construct
  MOD_RES 1: note = N-terminal myristol
  MOD_RES 11: note = phosphorylated serine
  MOD_RES 12: note = C-terminal -NH2
GKEAPPAPPQSP